SEQUENCE LISTING (128 sequences)

SEQ ID NO: 1 - DNA, 3063 bp, Brassica napus
atgtcgttaactcgacttatcttcatgatgtacgtactcgtctctctcatcctcttcggcttcgcttcctctcagaagttagccagagatgaagtggacgttttaagagccgtagcgaaggtgttacaccaaaacaactgggatttcagtgtggatccatgcgacgtctcttcaaccgtaggaggatggagaactcatgacaccggcgataacttcgaaaacaatgttacttgtaactgcttctcctccgtttgccatgtcaccagcatagttgtcaagggacaaaatcttaatggatctcttcctaaagagtttgcaggacttccttcactgcaggagattgatctgtctagaaactttctcagtggctcaattcctcccgaatggggagccttgccacttacagacgtttccttacttggaaaccgaataactggtccaatcccaaaagaaattggaaacattacaagccttatattccttgccttggaattcaaccagatttcaggtaaattacctccagagctcgggaatctaccaaacattcgaaggctgcttcttagctcgaacaacttgagtggagacatcccaagtacattctccaaacttacaacattgactgatttccgtataagtgacaatcagctaaccggtacaataccagattttacccagaactggacaaaccttgaaaaattggttattcaagcaagtggtttaatcggaccaattcctagtaccattggttctcttacaaagttaacagacttgaggatcagtgacctgagcggaccagagtctccatttccgccactacaaaacatgaaatcgttgcagacattgattcttaggaactgcaatgttacaggagagttaccagcgtatcttggaccgatcacatcattaaagctcttagatcttagctttaataagctaagtggaccggttccagtagcatatagagctctttccaacctagattacatatattttacaagtaacatgttaagtggggaagtgccaacttggatggtagacaaaggagataagattgatcttacttacaatgactttaccaatgatctaagaaccgcagaatgtcagaagaatgctgtgaatatgttttcaagcacaagccctttagtggcaaataaccactcaaacgtttcgtgtctgagtagctataaatgtcccaaaactttctatggacttcatataaactgtggtggtgctgaacttacaatcaatggcactaagtatgatagtgatacatcggagggagaaatcaactacgacactagatacggttggatttctagcaacacggggtacttcttggatgatgagcggtctcccaaaggagaaaccttatgggaaaataagtcagagcttacgataggagatcctggtctttatacacatgcacgtctatctgccatttccctcacttactacgcattttgtcttggacaaggaagctacactgttaatctccacttcgctgaaattatgttcactggcaaccatacatttagcagtttggggagacgattctttgacgtatacgttcagggaaagcttgaggttaaagatttcaatatagtagatgaggcaaaaggtgttggaagagctgtggttatgaatttcccggtcatgattacagatgggaaacttgaaataagattgttctgggctggcaaaggaactcaaggtcttcctacaagaggtgtatatggtgctctcatatcagctgtatctgtagatccaaatttcattccacctaaggaagctggcactggaagtggtggaagctctattggcactttggttggtgctgtagttgcttcgacggtgtttcttgtgcttttaatcggtggtttcttatggtggagaggttgcttaagacccaagagtcagatggaaaaagatttcaagaacttggatttccagataagttcattttcgttgaggcaaatcaaagttgctacaaacaactttgatccagcaaacaagattggagaaggtggtttcggtcctgtacacaagggaacgttgacagatggaaccgtaatggcggtgaagcagctatcatcgaaatcaaaacaagggaacagagagttcttgaacgagattgctatgatttctgctctacaacatccacatttggttaaactatatggatgttgtgtcgaaggtgaccagctcttgctagtctacgagtacttagaaaacaacagtctcgctagagcacttttcggtcctcaagagactcaaatacgactagattggccaactaggcagaagatttgcgttggaatagctagaggtttagcttatcttcatgaggaatcaagactcaagattgtacacagagacatcaaagccactaatgtcttgctagataaggaactcaacgcaaagatttcggatttcggtcttgctaagcttgatgaagaagaaaacacacacatcagcacacgagtcgcaggaacatacggatacatggctccagaatacgccatgaaaggtcatttgacagataaagcagacgtctatagttttggagttgtggccctagaaatcgttcatggaagaagcaacacgatcacacgttccagagtcgagaccttcaaccttcttgactgggtacacgttctaagggagcaaaacaaactgatggaagtagttgacccgaggctgggaacagattacaacagagaagaagcaatgaccatgatccagatagggatcctctgcaccagtcaagtttcgtcggaaagaccatccatgtcgacggtggtgagcattctcgaaggaagttccacggtgaatgttgagaagcttcttgaagcttccttcaacaaaggaagcgagaaagataacgagagcgtgagggcgatgaagaagcattacgcgatgataaatgaagaggagatgaatatgttggatcagactatcagcaccgacgggccattcacgtcgtcttctacttccacggccaacgccagtgatctttaccctcttaagcctgattctgcttattggaactctagggccgtttag

SEQ ID NO: 2 - protein, 1020 aa, Brassica napus
MSLTRLIFMMYVLVSLILFGFASSQKLARDEVDVLRAVAKVLHQNNWDFSVDPCDVSSTVGGWRTHDTGDNFENNVTCNCFSSVCHVTSIVVKGQNLNGSLPKEFAGLPSLQEIDLSRNFLSGSIPPEWGALPLTDVSLLGNRITGPIPKEIGNITSLIFLALEFNQISGKLPPELGNLPNIRRLLLSSNNLSGDIPSTFSKLTTLTDFRISDNQLTGTIPDFTQNWTNLEKLVIQASGLIGPIPSTIGSLTKLTDLRISDLSGPESPFPPLQNMKSLQTLILRNCNVTGELPAYLGPITSLKLLDLSFNKLSGPVPVAYRALSNLDYIYFTSNMLSGEVPTWMVDKGDKIDLTYNDFTNDLRTAECQKNAVNMFSSTSPLVANNHSNVSCLSSYKCPKTFYGLHINCGGAELTINGTKYDSDTSEGEINYDTRYGWISSNTGYFLDDERSPKGETLWENKSELTIGDPGLYTHARLSAISLTYYAFCLGQGSYTVNLHFAEIMFTGNHTFSSLGRRFFDVYVQGKLEVKDFNIVDEAKGVGRAVVMNFPVMITDGKLEIRLFWAGKGTQGLPTRGVYGALISAVSVDPNFIPPKEAGTGSGGSSIGTLVGAVVASTVFLVLLIGGFLWWRGCLRPKSQMEKDFKNLDFQISSFSLRQIKVATNNFDPANKIGEGGFGPVHKGTLTDGTVMAVKQLSSKSKQGNREFLNEIAMISALQHPHLVKLYGCCVEGDQLLLVYEYLENNSLARALFGPQETQIRLDWPTRQKICVGIARGLAYLHEESRLKIVHRDIKATNVLLDKELNAKISDFGLAKLDEEENTHISTRVAGTYGYMAPEYAMKGHLTDKADVYSFGVVALEIVHGRSNTITRSRVETFNLLDWVHVLREQNKLMEVVDPRLGTDYNREEAMTMIQIGILCTSQVSSERPSMSTVVSILEGSSTVNVEKLLEASFNKGSEKDNESVRAMKKHYAMINEEEMNMLDQTISTDGPFTSSSTSTANASDLYPLKPDSAYWNSRAV

SEQ ID NO: 3 - DNA, 2013 bp, Brassica napus
atgaagcagagaagtttgtttttaattctctgcttcgcccacataagcgttggcgatgctttggtttcagctaaaacatgtatggataataatgggtatttcaggcctaacggtacctatgacgctaatcgccgtcacatcctctcgtcacttccttccaacgtcacgtctcaagagggcctcttcttcaacagttccatcggacaagaacccaaccgtgtccatgcagtagggatgtgcatcccgggatcaatactagaggactgctttgcttgtatcaagtccgcgtgtgatgatttgatacaaaattgtcctaaccaaacaaacgcattttcatggcctggtgagcccaatctttgctatgtgcgctactccaacacttctttctcaggatctgcggttctggacccgcggccattgctatacagctcaaatgatatcaactcaaatctaacagagttcacaagaatatgggaagacttagttgttcgtatgattgatgcagcctccacagcaaaaagcacaccatcctctagtaataacttttacaaagctgatattgcagtcgtgacagcttccgataacatatacgctttaatgcaatgcacgccagatctttcctctggtgattgtgataactgtctccgacagagtgcaagggactacgagtcctgctgtggtcagaagcaaggaggtgttgttatgcggccaagctgcttcttcaggtggcagttagctacatactctaaggcttttggtaatattacggtgacttatcctcctcctcctcctcctcctcctcctgtggccacgcttcaacctgtagatgaaaatgaagatagcaaaggattctcatctggagttgtcgcggcatttacagttccaattgtcgttacggtctttatacttctcgttctaggattttttcgttgtcggaggagaaaatcaatgcaaagagttgaatctgataatgatatctcaactccacaatcatcccaatacgactttaaaacaattgaagctgcaacaaagaagtttttgatgagtaataagcttggtgaaggtggattcggcgaggtttacaagggtacactttcaaatggaactgaagtggctgtgaagcgactgtcgaaaaagtcaggacaaggaataagagagttcaagaacgaggctgttcttgtctcaaaacttcaacacagaaatttggtcaggcttcttggattctgtttggagggagatgaaaagattctgatctatgagtttgtccccaacaaaagccttaactatttcatatttggctttgaaaagcaaattcagctagattggagtcagcggtacaatatcatcgaagggattgctagaggaattctatatcttcatcaagattcacagctcacaatcatacaccgggacctcaaagccagcaacattctcttagatgccaatatgaacccaaagatttcagattttggattgtcaacgatctttgcaacagaacaaactcgaggcaataccaggagaattgctggaacatatggttacatgtctcctgagtatgcaatgcatggtcaatactccatgaaatctgacatttacagctttggagtcttagttcttgagattataagtggcaagaaaaacggcagcgtttaccggatggacgaaagtagtactgatggcaacttggtcacttatgcttggaggctttggaaaaatgggtcaccactagagctggtggatccagccatcggaaggaattaccagagtaatgaagtcactagatgcatccacattgcgctcttatgtgttcaagacaatccaataaagcgtccattgttatcaactatcatattgatgctcactagtaacactatcacactaccagtgcctcgtctaccacgtttcatcccgcggggcaggcacgaactggacctagaatcaagtcaatctacaggaaaatctgttgtttattctgtgaacgacgtgtcaattactgctttagagcctcgttaa

SEQ ID NO: 4 - protein, 670 aa, Brassica napus
MKQRSLFLILCFAHISVGDALVSAKTCMDNNGYFRPNGTYDANRRHILSSLPSNVTSQEGLFFNSSIGQEPNRVHAVGMCIPGSILEDCFACIKSACDDLIQNCPNQTNAFSWPGEPNLCYVRYSNTSFSGSAVLDPRPLLYSSNDINSNLTEFTRIWEDLVVRMIDAASTAKSTPSSSNNFYKADIAVVTASDNIYALMQCTPDLSSGDCDNCLRQSARDYESCCGQKQGGVVMRPSCFFRWQLATYSKAFGNITVTYPPPPPPPPPVATLQPVDENEDSKGFSSGVVAAFTVPIVVTVFILLVLGFFRCRRRKSMQRVESDNDISTPQSSQYDFKTIEAATKKFLMSNKLGEGGFGEVYKGTLSNGTEVAVKRLSKKSGQGIREFKNEAVLVSKLQHRNLVRLLGFCLEGDEKILIYEFVPNKSLNYFIFGFEKQIQLDWSQRYNIIEGIARGILYLHQDSQLTIIHRDLKASNILLDANMNPKISDFGLSTIFATEQTRGNTRRIAGTYGYMSPEYAMHGQYSMKSDIYSFGVLVLEIISGKKNGSVYRMDESSTDGNLVTYAWRLWKNGSPLELVDPAIGRNYQSNEVTRCIHIALLCVQDNPIKRPLLSTIILMLTSNTITLPVPRLPRFIPRGRHELDLESSQSTGKSVVYSVNDVSITALEPR

SEQ ID NO: 5 - DNA, 1368 bp, Brassica napus
atgctctcttctcttccttctaacgtcacagctaatgacggcgtctacacaacatcgaccggacaagatcccaacagagcgtacggtctagggatgtgtgtcccagtaaagacgtatctcttgaaaactgcactcattgtcttcaagagaacctgtttcattcggtgggaggtttatccgttcttggaccttttcgataatattgctcttgagaaagatggtaaaaagatttctacgggaactattgtggcgattgtcgttgttcccgttgtattgcttgccctaggatatgctctttgggagagaagagaagcattcaaagcatttacaaccgatactggggatgatattacaacttcaggttcacttcaattcgagtttaaagcaattgaagctgccacaagtaattttcataatactaacaagcttggtcatggtggatttggtgaagtttacaagggaacgttaccgaatggaacacatgttgctgtgaagaggctgtctaaaatgtcaggacaaggtgaagaagagttcaagaacgaggtgtttcttgtagcaaagcttcaacataagaatcttgttaggcttcttgggttttctgtcaaaggagaagaaaagatattagtctacgagtttttgcctaacaaaagtctcgatcatttcctttttgaccctaagaagagtagtcaactggattggacaaaacgatacaacattatcgagggaattactcgtgggattttgtatcttcatcaagattcgcgactcaccattatacaccgtgacctcaaagcgggtaacattctcttagatgaagatttgaatccgaagattgcagattttggtgtggctagaaatttcagtgtggaccaaactgaagctaccacaggaagagttgttggaacattcggttacatgccacccgagtacgtgacaagtggacagttctccataaaatctgatgtgtatagctttggagtattggttttggagattatttgtggaaaaaagaatagtagcttcaacgagacagacggctcaatcagcaacttggttacatatgtgtggaagctttggaacaatgactcattgctggaaatgatagacccggccatggaggagaattatgacagatgtgaagtcattaggtgcatccacattggattgttatgtgttcaagaaaatcatgtggatcgcccaaccatgtctacaatctatcgtatgctcactaatgcttctattactttgcatatgcctcaaccacctggatttgtcttcacggtcagatccaagtcaaacccattagaggagagatcgcaatgtggtccgtctactagtatatcgattacgtgtgttagtcctcgttaa

SEQ ID NO: 6 - protein, 455 aa, Brassica napus
MLSSLPSNVTANDGVYTTSTGQDPNRAYGLGMCVPVKTYLLKTALIVFKRTCFIRWEVYPFLDLFDNIALEKDGKKISTGTIVAIVVVPVVLLALGYALWERREAFKAFTTDTGDDITTSGSLQFEFKAIEAATSNFHNTNKLGHGGFGEVYKGTLPNGTHVAVKRLSKMSGQGEEEFKNEVFLVAKLQHKNLVRLLGFSVKGEEKILVYEFLPNKSLDHFLFDPKKSSQLDWTKRYNIIEGITRGILYLHQDSRLTIIHRDLKAGNILLDEDLNPKIADFGVARNFSVDQTEATTGRVVGTFGYMPPEYVTSGQFSIKSDVYSFGVLVLEIICGKKNSSFNETDGSISNLVTYVWKLWNNDSLLEMIDPAMEENYDRCEVIRCIHIGLLCVQENHVDRPTMSTIYRMLTNASITLHMPQPPGFVFTVRSKSNPLEERSQCGPSTSISITCVSPR

SEQ ID NO: 7 - DNA, 1863 bp, Brassica napus
atgatcatcctcgcttcttctcttctttttcatcagaccctcgaagctgttaacggcgcccggtgttttggaagcttagacagcaacagcagctacgcacagaatcgccgtgatcttttctctactcttgctaatgatgtcgtcactaacggcggattctacaacgcttcactcggccaatatcccaacacagtttacgctcttggcttctgcgaaaaatactacgagcaaaaagcttgtctccgttgtctcgaaagcttggctctggatacagaaacgagttgtggaaacatcacgaaatccttcgtttggagcagtgacgatgaagaccgtttttggtgccttatacgttcctcaaaccaaccttttgggaacttggagcttatacctcctttaatagaggcagatccagatcatattgagccatctaaagacatgacacttttcatgcaacagtgggaatcagcggttaataagaccctcgagactgccacacaagctaatacttcctcggtacataagtactatagtgccatacacgcccatttcacagagtttccgaatgttaacatggcgatgcaatgcacacccgacataacttcccaagattgcaagcaatgtttgggggattgtgtggagtactttagagaacagtttaggggaagagcagggggcatggctagttttccgagctgtttattcagatgggataacgttacaagtatgcctgcacttcctcgacctccggctcaagaaaagcggccgagctctataccagaaaagaaagcgttgcaaaattattttggaattatcacgataattgtggttctcactttcattactcttttggtatttattggtttctacaaagtaagagctcggaggagaaaattaaacaagggaataaatcttggttgtgcagaatactctaattcagatggtcaatttatgttactgttcaatcttgatatgatctcaatggcaacggctgatttttcgcctgaaaataagcttggccaaggtggttttggtacggtctataagggtatattactaaacgggatagagatagcggtaaagagattaaccagaggttcagaaggaggtatagagtttaggaatgaggtttcaatcttgacaagagtccagcataagaatcttgttaagcttcttggtttctgtaatgaaggagatgaaaagattcttgtctatgagtttgtccccaactcaagtcttgaccgctttatcttcgatgaagagaagcgctcgcttcttacatgggaagtgaggtttaaaattatagaaggaattgctcgaggtcttgtttatctccatgaagattctcagctgaagattattcaccgagacttgaaggcaggcaacatccttttagatgcagagatgaaccctaaagttgccgattttggaacagctaggctgtttgacactgatgagactcgagatgaaactaaacgaatcgctggaacccgtggatatatggctcctgaatacctgaattatggacaaatctcagctaaatctgatgtatatagcttcggtgttgtgcttctagagatgataagtggtcaaagaaataatagctttgaaggagaaggaattgcagcttttgcacggaagagatgggctgaaggaaggcctgaggttataattgatcctttattgatagagaatccgagtaacgagatcataaagttgatccacattggtctgttgtgtgctcaacagaatgcaacaaaaagaccaaccatgagcactgtaatagtttggcttggtagtcattcccaatctgaagatgatacaatgtcaatgagcaatgtcttcacggagttgagttgtcgttga

SEQ ID NO: 8 - protein, 620 aa, Brassica napus
MIILASSLLFHQTLEAVNGARCFGSLDSNSSYAQNRRDLFSTLANDVVTNGGFYNASLGQYPNTVYALGFCEKYYEQKACLRCLESLALDTETSCGNITKSFVWSSDDEDRFWCLIRSSNQPFGNLELIPPLIEADPDHIEPSKDMTLFMQQWESAVNKTLETATQANTSSVHKYYSAIHAHFTEFPNVNMAMQCTPDITSQDCKQCLGDCVEYFREQFRGRAGGMASFPSCLFRWDNVTSMPALPRPPAQEKRPSSIPEKKALQNYFGIITIIVVLTFITLLVFIGFYKVRARRRKLNKGINLGCAEYSNSDGQFMLLFNLDMISMATADFSPENKLGQGGFGTVYKGILLNGIEIAVKRLTRGSEGGIEFRNEVSILTRVQHKNLVKLLGFCNEGDEKILVYEFVPNSSLDRFIFDEEKRSLLTWEVRFKIIEGIARGLVYLHEDSQLKIIHRDLKAGNILLDAEMNPKVADFGTARLFDTDETRDETKRIAGTRGYMAPEYLNYGQISAKSDVYSFGVVLLEMISGQRNNSFEGEGIAAFARKRWAEGRPEVIIDPLLIENPSNEIIKLIHIGLLCAQQNATKRPTMSTVIVWLGSHSQSEDDTMSMSNVFTELSCR

SEQ ID NO: 9 - DNA, 1914 bp, Brassica napus
atgggcaaattctcagctttgatgatcatcctagcttcttctcttctttttgtccttcaaaccctcgaagttgttaatggcgccaagtgttacggaagcctagccggcaacaacagctacgctcagaatcgcaaaaatctcttctctactcttactaataaagtccttgctaacggcggattctacaatgcttcactcggccaatatcccaacagagtctacgctcttggcctctgtgcaagaggcttcaagccaaaagtttgtctcagttgtctcgaaagattgagcctggaaacacaaagggattgtccaaacatcatggactcgttcgtttggggcggtgacgatgaagagcttgtttcttgtctcgtacgttcctcaaaccactcttttgggaacctcgagattagtcctcctaatatacggatgagtccatatcatatcaggccattgataaacatgacccttttcatgctagaatgggaatacacagttaataggaccctcgaggctgccacaaaagctcatgcctcctcggcacataagtactatagtgcctcgtacgccgagttcacagcgtttccgaatgtttacatgctgatgcaatgcacacccgacataacttctcaaggctgcaagcaatgtttggaagcttgtttgaaatactttagagaacagtttctgggaagaatagggggcatagctacttttccaagctgttatttcagatgggatctatatcctttccatggtgcttttctgaatgttacaagagttccggcacttcctcgacctccgcctcaggaaaaagggagctctataccagataagaaaggtaaaacaaatgacatgaatagaggaattatcacgataattgtggttcttactttcattcatcttttggtatttattggtttctacaaagtcatagctcggaggttaaaaataaacaacggaaaaaatgttggtggtgcagaatacactgattcagatggtcaatttatgttacggtatgatcttggtatgatcttatcggcaacggctgtattttcgcctgaaaataagcttgggcaaggtggatttggtactgtttataaggggaaactacttaacgggaaagagatagcagtgaagagattaaccagaggttcagaaggagatatagagtttaagaatgaggtttcactcttgacaagactccaacataagaatctggttaagctgcttggtttctgtaatgaaggagatgaagagattcttgtctacgagtttgttccgcattcaagtcttgaccgctttatcttcgatgaggaaaagcgttcgcttcttacatgggatgtgaggtttaaaattatagaaggaattgctcgaggtcttgtttatctccatgaagattctcagctgaagattattcaccgagacttgaaggcaagcaacatccttttagatgcagagatgaacccaaaagttgcagattttgggacagcgaggctgtttgatgccgacgagactcgagctgaaacacaacaaatagctggaacccgtggatatatggctcctgagtacctgaatcatgggcaaatctcagctaaatgtgatgtatatagcttcggtgttgtgcttctagagatgataactggtcaaagaaataatagctttgaagaaggaattgcagcttttgtatggaagagatggtctgaaggaaggcctgttgttataattgatcctttattggtagaaaatccgaatgttcagatcattaaatatatccagactggtttgttgtgtgttcaagaggatgcatcaaagagaccaaccatgagctctgtaatggtttggcttggcagtgagacaatcaccattccatcacctaaggctcctgattacacaaagagtttttctgatagactttag

SEQ ID NO: 10 - protein, 637 aa, Brassica napus
MGKFSALMIILASSLLFVLQTLEVVNGAKCYGSLAGNNSYAQNRKNLFSTLTNKVLANGGFYNASLGQYPNRVYALGLCARGFKPKVCLSCLERLSLETQRDCPNIMDSFVWGGDDEELVSCLVRSSNHSFGNLEISPPNIRMSPYHIRPLINMTLFMLEWEYTVNRTLEAATKAHASSAHKYYSASYAEFTAFPNVYMLMQCTPDITSQGCKQCLEACLKYFREQFLGRIGGIATFPSCYFRWDLYPFHGAFLNVTRVPALPRPPPQEKGSSIPDKKGKTNDMNRGIITIIVVLTFIHLLVFIGFYKVIARRLKINNGKNVGGAEYTDSDGQFMLRYDLGMILSATAVFSPENKLGQGGFGTVYKGKLLNGKEIAVKRLTRGSEGDIEFKNEVSLLTRLQHKNLVKLLGFCNEGDEEILVYEFVPHSSLDRFIFDEEKRSLLTWDVRFKIIEGIARGLVYLHEDSQLKIIHRDLKASNILLDAEMNPKVADFGTARLFDADETRAETQQIAGTRGYMAPEYLNHGQISAKCDVYSFGVVLLEMITGQRNNSFEEGIAAFVWKRWSEGRPVVIIDPLLVENPNVQIIKYIQTGLLCVQEDASKRPTMSSVMVWLGSETITIPSPKAPDYTKSFSDRL

SEQ ID NO: 11 - DNA, 1095 bp, Brassica napus
atgaaaacttcccgttttacccttccctttcttctcatcgccttctccttcctcctcaccctctccgcccaaatgaccggaaacttcaaatgcggcgaaccaggtgactcaccctccacgtgtcgctccctcgtcggttactcaagcaagcaagccacaacctatggcaatatccaaaccctcttcgccgtcaagaagctccgttcaatcctcgaagccaacaacctcccactctccaccccacccgctcagggcgtgaacccgaaccaggtcgtacgcgtcccgatcccttgctcttgctccaacggaaccggcgtatcgaaccggactccggtttacaccgtcaagaagggagacatgctcttcttcatcgcatctgagatattcggagggttggttcgataccagaggatcagtgacctgaacaagattcccgacgcaagcgaaatcgatgtcggtcagaggttttggatccctttgccttgtagctgcgatgaagtaaacggtcaagatgttgtccactatgcacacgtggtgaaatccggaagctccctcggtgagatcgcttctcagtttggaactgacaacaggacgttggctcagctcaatgggatctccggcgatgctcagctcctcgctgactaccctcttaacgtccctctcagagcttgtaactcttccttgagggaggagtcgttggacgccaaaatgcttctacctaacagctcatactccatcactgcaaacaattgcatcaggtgttcttgtcaagcttcaaacaattggactttaagctgtgaagcctctcagcttaagccttcgtcgacctggaaaacttgcccgtctccacagtgtgaaggagcagagagtttgtttgtaggtaacacgacaaatacctcttgcggacctcgttcttgcgcctatgctggttactccaaccagacaatattcacagccctttctccagatccctgttcaggttctggtggtaatagttctggacctcctggcaactatgcttcaacgttcagctcaagtttcagtttcgcgatggtgttaattcagtgtgctctgatctgtctatgtcttctctag

SEQ ID NO: 12 - protein, 364 aa, Brassica napus
MKTSRFTLPFLLIAFSFLLTLSAQMTGNFKCGEPGDSPSTCRSLVGYSSKQATTYGNIQTLFAVKKLRSILEANNLPLSTPPAQGVNPNQVVRVPIPCSCSNGTGVSNRTPVYTVKKGDMLFFIASEIFGGLVRYQRISDLNKIPDASEIDVGQRFWIPLPCSCDEVNGQDVVHYAHVVKSGSSLGEIASQFGTDNRTLAQLNGISGDAQLLADYPLNVPLRACNSSLREESLDAKMLLPNSSYSITANNCIRCSCQASNNWTLSCEASQLKPSSTWKTCPSPQCEGAESLFVGNTTNTSCGPRSCAYAGYSNQTIFTALSPDPCSGSGGNSSGPPGNYASTFSSSFSFAMVLIQCALICLCLL

SEQ ID NO: 13 - DNA, 2829 bp, Brassica napus
atggcgttgttgtctcaaagagtctatctatatcagctccttgctgcgtttttctgcctgcttcttcttcttgcctatgcacgaacagacccctctgaagtcactgctttacgatcagttaagagaagtttagttgatcctaaagattatttgagaaactggaacagaggagacccttgcaggtcaaattggacaggagttatctgctccaatgagattggaactgacgaatatcttcatgtccgagaactcctactgatgaatatgaatctctccgggagtttatcaccagagctccgaaaattaggtcatctcgagatattagatttcatgtggaacaatataagtggttcaattccaaaggagatagggcagatatcatccctgctactcttgctgctgaatggaaacaaactttcagggcctttaccaagcgaactaggctatctttcgaacttgaacaggttccagattgatgaaaataatatcacaggaccaataccaaagtccttttccaacttgagaaaagtgaaacatctgcacttcaacaataattcgttgaccggccagattccagttgagctttccagtcttacaaacatctttcacgtgctgctagacaacaacaatttatccggagatctcccaccgcaactctctcaattgccaaacctgcaaatcctgcaacttgataataacaacttcagtggatcagatataccagcttcctatggaaacttttctagtatactgaagctgagtcttagaaactgtagtttgaaaggggctcttcctgattttagcaggatacgtcgtctcaaatacttggatcttagctggaatgagctcacaggacctataccttcttctaatctttctgaggatgtgacaaccattgatttatcaaacaacatactaaacggatccattccgctgagcttctcaaatctccctttgcttcagatgctatcgatgaagaacaatatgttatctggatctgtccctgattccctctggaagaatatctcattacggaagaaagctagacttctactggatctcaggaataatagtctgtcacatgttgaaggagatctaactcctccagaaaatgtcactttaaggcttgatggtaatccgatatgcaagaacggaagcataacaaacgcaggcctcttctgcgaatccataggaaaagcgtggacatcaccaccaccaccagcaaccaactcaacagactgtcctcctctagcctgtcccactcctgatttctacgaatacagtccagcatccccgttacgatgcttttgtgcagcgcctctcaggattggataccgtctgaagagtcctagcttctcatacttccctccatatattgatcagttcggggagtatgttaccgattttcttcaaatggagccctatcaactctggatcgactcttatcaatgggagaaagggcctcgtctgaggatgcatctgaaactttttccgcaagttacaagaagcacatttaacacgagtgaagttgtgagaatcagaggcatattcgcatcatggagatttcccgggagtgacttattcggaccatatgagctgctcaacttcactctgcaggggccatattcatatgtaaacttgagcagtgaaaggaaaggcgttagctggggacgtttggcagcaataactgcaggggctgtggtgactgctgttgcaatctccgccttggttgccgccttgttgcttagaagatacagcaagcacgagcgtgagatatctagaagacggtcttcctcgaaagcttccttgatgaattctgggataagaggcttcaggttcaaagaactagcagaggccacggacgattttagcagctcagctttggttggacgcggaggctacggaaaggtttatagaggtgtgttatctgacaaaacggttgctgccataaagcgagctgatgaagggtctttgcaaggtgagaaggagttcttgaatgaaatagagttgctctccaggttgcatcatcggaatctagtgtcccttatcggttattgtgacgaagaaggggagcagatgctggtgtacgaattcatgccaaacggcacccttcgcgactggctctctgctaaagggaaggagacattgagctttgggatgagggttcgtgttgctttgggagcagccaggggtattctctaccttcacacagaagccaacccaccagtcttccaccgagacatcaaagccagtaacatcctcctcgacctcaacttcaatgccaaagttgcagactttggactgtcccgtcttgctcctgctttggaggaggaagaggatgtgcctaaacacgtatccacggttgttcgtgggactccagggtacctggatccagaatacttcctgacgcacaagctgacggacaagagcgacgtgtacagcatgggggttgtgttcttggagctgctgactggtatgcacgccatatcccatggcaaaaacattgtgcgagaggtgaagacggcggacatgatggtgtcgctgatagacaagagaatggaggcgtggtcgatggaaaccgctgaaaggtttttctcattggcactgaggtgtagccacgactcgcctgatatgaggcctgcgatggctgaggtggtgaaagagctggaggcactcctccccgacaaggaaggaaaaatggagacgaagtcgtcctcctcggtgctgtcaacatcttcttcgaatgtaacaagagacctttacgagtcggcgagccttttaggaagcgatctcagcagcggcgttgttcccagcatcgctcctcgctga

SEQ ID NO: 14 - protein, 942 aa, Brassica napus
MALLSQRVYLYQLLAAFFCLLLLLAYARTDPSEVTALRSVKRSLVDPKDYLRNWNRGDPCRSNWTGVICSNEIGTDEYLHVRELLLMNMNLSGSLSPELRKLGHLEILDFMWNNISGSIPKEIGQISSLLLLLLNGNKLSGPLPSELGYLSNLNRFQIDENNITGPIPKSFSNLRKVKHLHFNNNSLTGQIPVELSSLTNIFHVLLDNNNLSGDLPPQLSQLPNLQILQLDNNNFSGSDIPASYGNFSSILKLSLRNCSLKGALPDFSRIRRLKYLDLSWNELTGPIPSSNLSEDVTTIDLSNNILNGSIPLSFSNLPLLQMLSMKNNMLSGSVPDSLWKNISLRKKARLLLDLRNNSLSHVEGDLTPPENVTLRLDGNPICKNGSITNAGLFCESIGKAWTSPPPPATNSTDCPPLACPTPDFYEYSPASPLRCFCAAPLRIGYRLKSPSFSYFPPYIDQFGEYVTDFLQMEPYQLWIDSYQWEKGPRLRMHLKLFPQVTRSTFNTSEVVRIRGIFASWRFPGSDLFGPYELLNFTLQGPYSYVNLSSERKGVSWGRLAAITAGAVVTAVAISALVAALLLRRYSKHEREISRRRSSSKASLMNSGIRGFRFKELAEATDDFSSSALVGRGGYGKVYRGVLSDKTVAAIKRADEGSLQGEKEFLNEIELLSRLHHRNLVSLIGYCDEEGEQMLVYEFMPNGTLRDWLSAKGKETLSFGMRVRVALGAARGILYLHTEANPPVFHRDIKASNILLDLNFNAKVADFGLSRLAPALEEEEDVPKHVSTVVRGTPGYLDPEYFLTHKLTDKSDVYSMGVVFLELLTGMHAISHGKNIVREVKTADMMVSLIDKRMEAWSMETAERFFSLALRCSHDSPDMRPAMAEVVKELEALLPDKEGKMETKSSSSVLSTSSSNVTRDLYESASLLGSDLSSGVVPSIAPR

SEQ ID NO: 15 - DNA, 1338 bp, Brassica napus
atgtggtatctccccacttcttgtcttgtctttttcttcctcttctccttattgcaccatctttcttgtgcttcaactaaaaaagaacttgggtggtgtgaggctatttttgagtgtgggaacatcaccgctggtttccccttctccggtgggagtcgtccccagttttgcggccatccatcgctggagcttcactgctttaacaacaagacatctataataatctctgatcatctgtacgatgttctccatatagatcaaatatctaagactattaaacttgcaaaagcagagcttcgaggttctttttgcaccgctacattcacaaccacaactttgcctcccgaaatctttgagctttcaacaacctctcagagcctaacagttttctacctctgccaccgtaaccttcgttataactcgagttatttatgtcctgacagaggttctatctcagtgtctcaaaaccttaattaccacaagtcctgccaagacagtttcaaaattaacgttccgaagagctacgtaccggaagagaaagagttgaatttaaaacatttggaaaatgctttgcatgaagggtttgatgtgaaagtgaagattgatgaacaaacatgtgaacaatgtttgtcctctcaaggaatctgcggcttcaataatacaacacagatctgctgcaagaatgactcatcatcaagatgcaatacactccatatacctcgtatagatgtacttcaccaacgctgcagtggcccctttaggtgtggcgatcagaaagaactctattaccctttctggagatcagagagagaaagctgcggccaccccgacttcaagctcgactgtagtggagaattcgctgaactgaacatttcctctgtgaagtacagaatcttaagtatgagctatggatctcctgtcattagtcttggcagatcggattatatcggccatctttgtcccgcagatactcgagatgcaccatttgaccaaagtgttcttcaaattacgcgtgataacgatctacttacattatattacgactgcaacagcttttcactaccagctactactggaagtaatttctttggagagcttggttgcgaggatgatataggcgatagaaaaagttactatgtgacaagaaacatgtcctcccctttacctaacgagatcagtggccttatagacaaggcaatttgcagaagagacgtaactattcctgtgtctataaatgctttgaaccaaatggaggggagtcctagtccggatagtttagagaaggctcttgagaaggtttcgagcttgaagttagtcctgattgttcacggtgcttaa

SEQ ID NO: 16 - protein, 445 aa, Brassica napus
MWYLPTSCLVFFFLFSLLHHLSCASTKKELGWCEAIFECGNITAGFPFSGGSRPQFCGHPSLELHCFNNKTSIIISDHLYDVLHIDQISKTIKLAKAELRGSFCTATFTTTTLPPEIFELSTTSQSLTVFYLCHRNLRYNSSYLCPDRGSISVSQNLNYHKSCQDSFKINVPKSYVPEEKELNLKHLENALHEGFDVKVKIDEQTCEQCLSSQGICGFNNTTQICCKNDSSSRCNTLHIPRIDVLHQRCSGPFRCGDQKELYYPFWRSERESCGHPDFKLDCSGEFAELNISSVKYRILSMSYGSPVISLGRSDYIGHLCPADTRDAPFDQSVLQITRDNDLLTLYYDCNSFSLPATTGSNFFGELGCEDDIGDRKSYYVTRNMSSPLPNEISGLIDKAICRRDVTIPVSINALNQMEGSPSPDSLEKALEKVSSLKLVLIVHGA

SEQ ID NO: 17 - DNA, 4398 bp, Brassica napus
atggaagggtcaagtaacggttctttgagattagtgttgttacatggtaacttagacgtttgggtgaaggaagctaaaaatcttcctaacatggatcgttttcggaggtacaagaagaacagtacaagtgatcccttcgtgactgtctctatcgcaggtgcaaagataggcacaacttttgtggtcgacaacgatgagaatcctgtgtggaagcagcatttctatgtaccggtggctcaccatgctaaggtggtaaagtttgtggtgaaagacagtgaccgttttggagcaaagttcataggagatgttggaatcccaaccgaggagttatgttcaggaaacacgatcgaagggctgtttccgatacttgatagtagtcggaagccatgtaaaaagggtgctgtgttgagtttggctattcaatacactccagtggaaatgatgaaattttaccaaatgggtgttggtaatgagtgcgaaggagttcccggtacatacttccctttgaggaaaggcggtagagttactctgtatcaagatgcccatgttgaagacgggacacttccgagtgtagatctcgatggtgggatgaagtatatacatggaaagtgctgggaggatatgtctgatgcgattagacaggcaaagaacttgatttatatcacaggttggtcagtttaccattcggttaggctggttcgtcgcaataatgatccgaccaatggtacattaggggatttgcttaaagaaagatctcaagaaggtgttagagtgttgctattggtgtgggatgatccaacttcaaggagctttctggggtacagaacacgcggatatatgaagacaagcgatgaggagactcgccatttttttaagaactcgtcggtgcaagttattatttgtcctagatctgggggaagaggtcttcatagctttgtaaaaaaaactgaagttcaaactatctacacacatcatcagaaaactgtgattgtagatgctgaggcagctcagggtcgaagaaagatcgtagcgtttgttggagggatagacgtgtgcaaaggacgttttgatacgcctaagcatcctctctttacgacattaaagactcttcataaagatgatttctataacaattgttttgggactactgaggatgatggaccaagacaaccgtggcatgatctgcacagcatgattgatggtccagcagcgtatgacgtgcttgctaattttgaacaacgttggctaaaggcttcagagaaaaggcacaggatttcgatacacagatcgtcttctgaggatgctttgcttaagattgacaaaattccaaatatcatgggactatcagaagcttcttttgttgatgataatgatccagagtcttggcatgttcaggtttttcggtccattgattcaacctcagtcaaagggtttccagaggactcaaaggaagctagtgcaaggaatcttcaatgtgggaagaatatacttatagacatgagcatacacacggcttatgttaaggccataagatctgctcagcatttcatttacattgagaaccaatatttttttggatcatcatttaactgggattcacataaaaccgttggtgctaataatctaatccctatggaaattgcgcttaagattgctaataaaattagagcaagagaaaattttgctgcttatattgtcattccaatgctgccagaaggtgatccaacgggtatcgttacgcagagcattttacaatatcagtataaaaccatgcaaatgatgtatctaactatctacaaggcacttgtggaagctgaacttgatggtcagtatgagccacaagactatttgaacttcttctgtcttggaagcagagaggttgctgatggaaacgttaacaacaacacaaaggaagaagatggcccacaggtggaagctttgaagagtcgaagattcatgatatatgttcattccaaaggtatgatagtggacgatgagtttgtcttaattggttctgcgaatatcaacgagagatccctggaaggatctagagacactgaaatcgcaatgggaggatatcaaccacaccactcatgggctaagaaaggttctcatcctcgtgggcagatctttggatacagaatgtcgctgtgggcggaacatctagggtctctagagaaaggtttcgaaaatccagagaacatggaatgcgtgagacgagttaggagattgagtgagcttaactggagacagtatgcagcagaggaagtcacagagatgacaagtcatcttctaaagtatcccgttcaagtcgatagggcaggcaaagtgagttctcttcctggatgcgagacattcccagatgtgtttgtggatcctgctagacacttgtgtcatcctgaccaaagggatgcgattctggatttcaagaacaaattccagattcaaaagccttgtcttgaccgacgtctaaagtcatgggagaataacagcgattgttgttcatgggacggtatcagatgtgatgccagtttcggggatgtgatcaagctgaaccttagtgacagttgcctccatggtcagttcaattccaacagtactatctttctgcttcagaggcttccttttttaacaactctcgacctttcaagtaatgagctcagtggtaatattccatcttcacttggaaacctttcaaagctaaccactcttgacctttcaggtaatgatttcagtggtgaaattccatcttcacttggaaacctctctaagctcaccactcttaataatctctcccaaaacaaatttactggtaaagttccatcttcacttggaaatcttttgaatctcactgagcttatcctttgtgctaacaactttgttggtgaatttccatcttcatttggaaacctttcacatctcactgttctcgacctctcacaaaacaatttcgttggtgaaatctcatctttttttggcagttcggatcatctcactgacatggatgttggaaacaacatccttcatggtaactttcctcttaaactactaaatctaacaaagctgatttccatatcaatcagcggaaatcaattctcgggcatgcttccacctaacatgggctcactctccaacttggagttcttttacgtagaacacaacacttttactggacctatcccttcttcttctctctttaccattccttctttaacttatattgatttccttggcgataacaacttcatgggatcaatcccccgatccatctccaagttggtcaaccttgacacacttgatctttctcatctcaacacccaaggatgcggtatcttcgagtttccggagctcttacgaacccaacgcaacatgaaaacactagacatttctaacaacaaaatcacaggtcaagtgcctggatggttatgggagcttccaattattgaatacgtgagtacttccaacaacacattgattggttttgaaagcccaacgaaacatgggatatcttatgtccggaaaacatctctgagttacttgtctggcgccaacaacaatttcacgggcaagatccctagtttcatatgtgagttgcgctctctaaccactctcgatttgtctaacaacaaattcagtggctcattccctagctgtctgggaaattttagcagaagtcttgaagttctaaatcttgacgtcggtcataaccgtctggtgggaaagcttccaagatcattcatccgtatccattttcttcaagttctgaatgtggagatcaacaaaatcagtgacacgtttcctttctggttgaggtctctgccggagttacaggttcttgtccttcgctccaacgcatttcatggagagattccaaggtccattggtctattgaaagagcttcatgtgctcaacttttcaaacaatgctttcactggccacatcccatcatcactgggaaacctggcagagcttgagtcactggatgtttcgcaaaacaatctttcaggagagattccacaagagcttgggagcctctcgttccttgcttacatgaacttctctcataaccagcttataggtctagtgccagggggaactcagtttctgacgcagccttgttcttctttcgaggacaacccgggactgtatggtccttcacttgatcaagtttgtggttgtatccacgcgacaacatgtcaacaatctgaaacgccagatccagatgaagatgaagatgaagaagaggtgctgagttggatagcagctgcaataggatttgtaccaggtgttttctttggattgacgatgggatacatactggtttcctacaaaccagagtggttcatgaaagcttttgtcccaaacaaacgcagacaccacaactcattgaagaggtga

SEQ ID NO: 18 - protein, 1465 aa, Brassica napus
MEGSSNGSLRLVLLHGNLDVWVKEAKNLPNMDRFRRYKKNSTSDPFVTVSIAGAKIGTTFVVDNDENPVWKQHFYVPVAHHAKVVKFVVKDSDRFGAKFIGDVGIPTEELCSGNTIEGLFPILDSSRKPCKKGAVLSLAIQYTPVEMMKFYQMGVGNECEGVPGTYFPLRKGGRVTLYQDAHVEDGTLPSVDLDGGMKYIHGKCWEDMSDAIRQAKNLIYITGWSVYHSVRLVRRNNDPTNGTLGDLLKERSQEGVRVLLLVWDDPTSRSFLGYRTRGYMKTSDEETRHFFKNSSVQVIICPRSGGRGLHSFVKKTEVQTIYTHHQKTVIVDAEAAQGRRKIVAFVGGIDVCKGRFDTPKHPLFTTLKTLHKDDFYNNCFGTTEDDGPRQPWHDLHSMIDGPAAYDVLANFEQRWLKASEKRHRISIHRSSSEDALLKIDKIPNIMGLSEASFVDDNDPESWHVQVFRSIDSTSVKGFPEDSKEASARNLQCGKNILIDMSIHTAYVKAIRSAQHFIYIENQYFFGSSFNWDSHKTVGANNLIPMEIALKIANKIRARENFAAYIVIPMLPEGDPTGIVTQSILQYQYKTMQMMYLTIYKALVEAELDGQYEPQDYLNFFCLGSREVADGNVNNNTKEEDGPQVEALKSRRFMIYVHSKGMIVDDEFVLIGSANINERSLEGSRDTEIAMGGYQPHHSWAKKGSHPRGQIFGYRMSLWAEHLGSLEKGFENPENMECVRRVRRLSELNWRQYAAEEVTEMTSHLLKYPVQVDRAGKVSSLPGCETFPDVFVDPARHLCHPDQRDAILDFKNKFQIQKPCLDRRLKSWENNSDCCSWDGIRCDASFGDVIKLNLSDSCLHGQFNSNSTIFLLQRLPFLTTLDLSSNELSGNIPSSLGNLSKLTTLDLSGNDFSGEIPSSLGNLSKLTTLNNLSQNKFTGKVPSSLGNLLNLTELILCANNFVGEFPSSFGNLSHLTVLDLSQNNFVGEISSFFGSSDHLTDMDVGNNILHGNFPLKLLNLTKLISISISGNQFSGMLPPNMGSLSNLEFFYVEHNTFTGPIPSSSLFTIPSLTYIDFLGDNNFMGSIPRSISKLVNLDTLDLSHLNTQGCGIFEFPELLRTQRNMKTLDISNNKITGQVPGWLWELPIIEYVSTSNNTLIGFESPTKHGISYVRKTSLSYLSGANNNFTGKIPSFICELRSLTTLDLSNNKFSGSFPSCLGNFSRSLEVLNLDVGHNRLVGKLPRSFIRIHFLQVLNVEINKISDTFPFWLRSLPELQVLVLRSNAFHGEIPRSIGLLKELHVLNFSNNAFTGHIPSSLGNLAELESLDVSQNNLSGEIPQELGSLSFLAYMNFSHNQLIGLVPGGTQFLTQPCSSFEDNPGLYGPSLDQVCGCIHATTCQQSETPDPDEDEDEEEVLSWIAAAIGFVPGVFFGLTMGYILVSYKPEWFMKAFVPNKRRHHNSLKR

SEQ ID NO: 19 - DNA, 609 bp, Brassica napus
atgaatcctacgttttacttcgttcttgccttaaccatagttttggccacaaacacatatggtgcagttcttgacatcgacggcgacatcattttccgcggcagttactatgttctcccagtcatccgcggaagaggaggggggctaactctacgcggccgcggtggcgagctatgtccttacgacatcgtgcaaaaatcatccgaggttgatgaaggtattcccgttaaattctcaaactggagacctagagttgcgttcgttcccgagtcgcaagaccttaacatcaagacggacgttgaagctacgatatgcttccagtcaacatactggcgagtcggtgagtttgacgaggagaggcagcagtatttcgtggtggctggtctacaagacgactcacccaacagtttctttcagatcgaaaactctggagatgatgcttacaagtttgtgttctgtcctaagactggtgattctggtcgtcaatgcacgaatgtcgggatattcgtggacgaaataggcgttcggcgtttggctctaagctctgagccgttcttggttatgttcaagaaagctaatgttactgagatttcgtccaagactatgtga

SEQ ID NO: 20 - protein, 202 aa, Brassica napus
MNPTFYFVLALTIVLATNTYGAVLDIDGDIIFRGSYYVLPVIRGRGGGLTLRGRGGELCPYDIVQKSSEVDEGIPVKFSNWRPRVAFVPESQDLNIKTDVEATICFQSTYWRVGEFDEERQQYFVVAGLQDDSPNSFFQIENSGDDAYKFVFCPKTGDSGRQCTNVGIFVDEIGVRRLALSSEPFLVMFKKANVTEISSKTM

SEQ ID NO: 21 - DNA, 2289 bp, Brassica napus
atgatgaatccgatccaatcttttttcttcttctttctttcccttatatgcagcactctttcttctcctacgctccaaatggatgctcttttggagttcaaaaatgagttcccgttcagtgcaccaaacccaaatgcttcttttgatttctcttttagttggtggaatacgagcactaatcactgttcctggaaaggtgtcacatgcaatgctaattccggtgcggtgatatcacttgtccttgaagacatcgttctcaatggctctttgaaagcaaacactagtctgttaaaactccaacatcttcagaaccttaccctttgcaattgcattctcagaggagagattccttcttcactaggaaacctttctcatctcatgcagcttgacctttttaacaatgatctggtaggtgaaattccgacttcactaggaaacctaaaccagctaacatttatgagcttttcctcaaacaacttgactggtcacgttcctagttcatttgccaatttcaataagctgtccaatttagatctctcaagaaatcaattctccggtggagatttacctcttatactatcaaatttaaccagcttgtccgaactagacctttccagtaatcacttcaaatccaagcttccatccaacatgagtcgactccataacttgaaagtatttgacgtgaaaagaaattattttctcgggaatgtccctacatccctctttatgattccaatgttactaggtgtttatttgcgtggaaaccaattcgaaggacccttagagtttgggaatacatccgcatcttctaggttagtcagcctagacctcagttctaacaacttccacgaaccaattccagaagatatatttagatttctcaaactcgaaactttagatcttagcaataacagtttcactggggcaatctctagatctatgtccaaattagtctccctcaggtttctagatctatcctacaataagatggaaggtcaagtaccgagtttcttgtggagcctagacaccttgacgctttctcataattatttcagtagcctcgaggaagttgtcgtcaatggaatattggttttcaaagttgatcttggttcaaattcactccaaggatcatttcccctatggatctgtaagtcaacattactcttattattagatctgtcaaacaaccatctctctggttccattccttcatgtttgatgaattccactgttaatatacttctaagaaacaacaatttgagcggattcctcccagatatctttgccaatgccaccgacgtactatacttagatgttagccgtaaccagttaatggggaagcttccaaaatcactgatcaactgcaagtatatgcattatctgaatctgaaaggaaacaagttcaatgacacgtttccatactggttgggatctttggtatcactacgtgttctgattctcggatcaaatgccttttatggtccagtctcttcacatgttgggtttccaagtctgaaaatcattgacatatcgcataatagcttcaatggaacattgccacaagattattttgtgaactggcttgaaatgtcaacagtgtgggtagattacgaaggtattcgaggttcgttatacatgggggcaatcaatcatacggattcgatggatatgatgtataaaggtgtagacacagagtttccactgatctttcttgggttcaaagccatcgatttttctggaaacaaattcactggacgaatccctaaatcagttggcttgttgaaggatttgcgtcatgtcaacttttcaagaaatgcatttaccggcagtatcccatcatccttggcaaaactaacaaacctcgaggcactagacctctctcacaataagctttccggtaatattcctcgtgatcttgccagactctccttcatgtcatacatggacttctcccacaaccttctccaaggtccagttccacgaagcactcagtttcaaagccaaaattgttcttcgttcgaagacaaccttggactctacggtctcgagacaatctgtggaccgatccatggtcctcatcctacaccgggagattcacatcaatccgaagaattttcatccgaagagtcagaagaagtgttgagctggatagcagctgcaatagccttcggacctggggtgttctgtggattggtgatcggatatatcttcttcacttcacaaaagcacatatggttcatggagaagttcggtcgaaactatccccgtggcatcatcactgttggctaa

SEQ ID NO: 22 - protein, 762 aa, Brassica napus
MMNPIQSFFFFFLSLICSTLSSPTLQMDALLEFKNEFPFSAPNPNASFDFSFSWWNTSTNHCSWKGVTCNANSGAVISLVLEDIVLNGSLKANTSLLKLQHLQNLTLCNCILRGEIPSSLGNLSHLMQLDLFNNDLVGEIPTSLGNLNQLTFMSFSSNNLTGHVPSSFANFNKLSNLDLSRNQFSGGDLPLILSNLTSLSELDLSSNHFKSKLPSNMSRLHNLKVFDVKRNYFLGNVPTSLFMIPMLLGVYLRGNQFEGPLEFGNTSASSRLVSLDLSSNNFHEPIPEDIFRFLKLETLDLSNNSFTGAISRSMSKLVSLRFLDLSYNKMEGQVPSFLWSLDTLTLSHNYFSSLEEVVVNGILVFKVDLGSNSLQGSFPLWICKSTLLLLLDLSNNHLSGSIPSCLMNSTVNILLRNNNLSGFLPDIFANATDVLYLDVSRNQLMGKLPKSLINCKYMHYLNLKGNKFNDTFPYWLGSLVSLRVLILGSNAFYGPVSSHVGFPSLKIIDISHNSFNGTLPQDYFVNWLEMSTVWVDYEGIRGSLYMGAINHTDSMDMMYKGVDTEFPLIFLGFKAIDFSGNKFTGRIPKSVGLLKDLRHVNFSRNAFTGSIPSSLAKLTNLEALDLSHNKLSGNIPRDLARLSFMSYMDFSHNLLQGPVPRSTQFQSQNCSSFEDNLGLYGLETICGPIHGPHPTPGDSHQSEEFSSEESEEVLSWIAAAIAFGPGVFCGLVIGYIFFTSQKHIWFMEKFGRNYPRGIITVG

SEQ ID NO: 23 - DNA, 1869 bp, Brassica napus
atgttcgcaggagtgttgtcagggagtatccttaccatagagaacaaatgtaatcagacagtttggccagtaatcttctcatgggaatcaaacctctccaccactggtttcgttctcagaagcggcgaggcgcgtgtcctgcaggcgccgtcttcatggtatggtcttatctcggctaggacgctctgctcaaccaactcgagtggttacttctcgtgcgccaccggagactgtgaatccggcttcatagaatgtcccggctcatattcttggtctgccgtgacgtatgtctactttagaatggaccacggcggagttagcagctacacgatcactgtcgagcacggttacaaccttcccttagtggtagtcccgtcacagcctagccagacatgtatcagcgccggttgtttggttgacttgaacaagacttgtccagaggatcttggttttttcaccggtgggaaacaaatcggctgcattagcgcgtgcagaaagtacaacaccaaagaaatttgctgcactaatgacttcaggtcaaagcaaagatgcgagaggacgatgtacacgaagaacttcgagcaagcttgcccactcacctatagctatgccttcgaagataataacagcaccatgacatgccctaagtcaactgacttcgtcctcaccttttgtccctcgtccattcccaataacacaagaagctccatgtctccatttgcaggacccaaaaataattctaaagggaagttaaaacccatactcggaggttcatcagctttagccgtgttgatcattgctgttgcggttgtggtaatggtgagagcaaagaatgcgagaagaaagcgtgattcaaattacgagaacattgaagcggttgtaatgttgaaacgatatagttatgcagacgtcaagaagatgacaaactcattcgctcatgttcttggaaaaggaggatatggaactgtctacaaaggaaaactacccgattcgagcggacaagatattgcactcaagatcttgaaagacccaaaagagaatggagaagacttcatcaacgaactagctagcatgagcatagcatctcatgtcaacatcgtttctctatttggattctgctatgaagggagcaagagagctatcatttacgagttcatgcctaatggatcccttgacaagtttatttccgaagatatgtcaacgaagatggactgcgaaacattatacaacattgcgttaggtgttgctcgtggcttagactacttgcacaatagttgtgtatcaaagattgtgcatttcgatataaagccacagaatatactcctagatgaagatttgtgcccgaagatttcggattttggtcttgctaagctttgcaagaaaaacgatagcattatatccatgttggacgcgagagggaccgttggttatattgctcctgaagtgttttccaagagttatggagcagtttcgcataagtctgatgtgtatagttatggaatggtggtgcttgagctcatcggggtgaccagtagagagagagctgaaacttctaggtctaatatgagtacaatgtactttccggattggatatatgaggatctggagaggaatgaaaatatgagagtaggagatcatgttattgaagaggaagaggagatagtgaagaaaatgacattagtgggtttgtggtgtattcagaccaatccatgtgatcgtccaccgatgaaaaaggttgttgaaatgttagagggaggtgtagaagctctagtggtcccacctaagcctctcttgactccagccataatggcttgggaaaccgatgaagagagtgaagagactaccagtcttttgacaccaagccactag

SEQ ID NO: 24 - protein, 622 aa, Brassica napus
MFAGVLSGSILTIENKCNQTVWPVIFSWESNLSTTGFVLRSGEARVLQAPSSWYGLISARTLCSTNSSGYFSCATGDCESGFIECPGSYSWSAVTYVYFRMDHGGVSSYTITVEHGYNLPLVVVPSQPSQTCISAGCLVDLNKTCPEDLGFFTGGKQIGCISACRKYNTKEICCTNDFRSKQRCERTMYTKNFEQACPLTYSYAFEDNNSTMTCPKSTDFVLTFCPSSIPNNTRSSMSPFAGPKNNSKGKLKPILGGSSALAVLIIAVAVVVMVRAKNARRKRDSNYENIEAVVMLKRYSYADVKKMTNSFAHVLGKGGYGTVYKGKLPDSSGQDIALKILKDPKENGEDFINELASMSIASHVNIVSLFGFCYEGSKRAIIYEFMPNGSLDKFISEDMSTKMDCETLYNIALGVARGLDYLHNSCVSKIVHFDIKPQNILLDEDLCPKISDFGLAKLCKKNDSIISMLDARGTVGYIAPEVFSKSYGAVSHKSDVYSYGMVVLELIGVTSRERAETSRSNMSTMYFPDWIYEDLERNENMRVGDHVIEEEEEIVKKMTLVGLWCIQTNPCDRPPMKKVVEMLEGGVEALVVPPKPLLTPAIMAWETDEESEETTSLLTPSH

SEQ ID NO: 25 - DNA, 495 bp, Brassica napus
atgatgaatcctatgttttactttcttcttgccttaaccgctgttttagccacgaccgcaaacgctggaccagttctcgacattgatggtgatatcatattccacggcagctactacgttatccccgtcatccggggccctgaaggtggcggcttaactctcaccccacgcaatggcaaccagtgtcccctctttatcggacaggagcgttcagaggtcgaaaggggcattcccgtgaaattctcaaactggaggtctagagttgggttcgttcccgaatccgagaacctcaacatcaagatggatgtcgaagctacgatctgcgctcagtcaacttattgggacggcaacgattgcatcgatgtcggtaaaaacgtggaaggtggcgttcagggtttggttgtatctaggccaccattcgctaccccattcgaggttgtgttcgtgaaagctactgagaaacagacttcatccaagaccatgtctattatctga

SEQ ID NO: 26 - protein, 164 aa, Brassica napus
MMNPMFYFLLALTAVLATTANAGPVLDIDGDIIFHGSYYVIPVIRGPEGGGLTLTPRNGNQCPLFIGQERSEVERGIPVKFSNWRSRVGFVPESENLNIKMDVEATICAQSTYWDGNDCIDVGKNVEGGVQGLVVSRPPFATPFEVVFVKATEKQTSSKTMSII

SEQ ID NO: 27 - DNA, 1986 bp, Brassica napus
atgactcttgggaccaacaggagaaccggagagaatctgaagcttacttcttggagaagctatcaagatccttcgacagggaactacacagccggtcttgctcctttaacgtttcccgagctcacgtttcctgagcttctgatttggaagaacaatgttccagtctggcgtagtggaccgtggaacggccaggttttcatcggtttaccggacgtggattctcttttgtttcttgatgggtttaatcttatcaatgataagcaaggaacattttcaatgtcatttgctaatgattctttcatgtatcactttaacttggatcctgatggagttatatatcagagagattggagtacttctttgagagattggaggatcggtgcaatgtttccatctacgtactgtgatgcatacggtatatgtggtccaaacggaagctgcagttcccgggaagatccgccttgtgaatgtgttaaagggtttgtgccgaggaacagcacagagtggaatgcaaggaattggagtaatggatgtgtgagaaaaggtcaattgcggtgcgagaggcagagcaatggaggaggaaaaggagatgtgtttgttagactgcagaagatgaaagtaccagtcaatgcagtacaatctgatgctaatgagcaagattgtcctaaacagtgtaaggataactgttcttgcactgcttatgcttttgatcggggaatcggatgcatgctttggagtggtaacttagttgatatgcaatcatccttgagaactggcattgatctttatattcgacttccacattctgaactcaaaacacatagcaatcgagcagttatcatcattgcacccgtgctaggcgttgcgttccttgctgctgtctgcgttcttttagcatgcaggaaattcaaaaaacgtccagatacaagtgcagagctaatgtttaagagaatggaagcacttacaagtggtaatgagactgcttctaaccaagtgaagctcaaggagcttccactctttgagtttcaagtgttagccacagcaactgatagcttctctctaagaaacaagctcggacaaggtggatttggtcctgtttacaagggaaaattgtcagaagggcaagaagttgcagtgaagaggctctcacaggcatcaggacaaggacttgaggagcttatgaacgaagtggttgtgatttccaagttgcaacatcggaatctagtgaagttacttggctgttgcattgaaggtgaagaaagactgttagtctacgaatatatgcctaataaaagcttggatgcctatctatttgacccattgaagcaaaagattcttgactggaagacacggttcaacataatggaagggatttgcagaggtctcttgtatcttcacagagattcaagactaaagatcatacacagagatctaaaagtcagcaacattttgttagatgacaatctgaatcccaaaatatctgatttcgggcttgcaagagtttttcgagcaaatgaagatgaagctaacacaagaagggttgttggaacatacggctatatgtcacctgaatatgcaatggaaggtttaatttcagaaaaatcagacgtttttagtttgggggttatatttttagagatcctaagtgggagaaaaaactctcacaaggaagagaataatctaaaccttttagcttatgcgtggaagctgtggaacgaaggtgaggctgcttctctagctgatccaaccatctttgataagtctttcgagaaagagataacgagatgtgttcagattggtttgttgtgtgcgcaagaagctgcaaacgatagaccaaatgtttcaaccgtgatctggatgctaactaccgaaaacacgaacctccaagagccaaagcagcgtgcgttaatagcaagaagaggatcttgtgaccagggtagtctctctaccaatgatttgagcctcacagctgtaacagggcgttag

SEQ ID NO: 28 - protein, 661 aa, Brassica napus
MTLGTNRRTGENLKLTSWRSYQDPSTGNYTAGLAPLTFPELTFPELLIWKNNVPVWRSGPWNGQVFIGLPDVDSLLFLDGFNLINDKQGTFSMSFANDSFMYHFNLDPDGVIYQRDWSTSLRDWRIGAMFPSTYCDAYGICGPNGSCSSREDPPCECVKGFVPRNSTEWNARNWSNGCVRKGQLRCERQSNGGGKGDVFVRLQKMKVPVNAVQSDANEQDCPKQCKDNCSCTAYAFDRGIGCMLWSGNLVDMQSSLRTGIDLYIRLPHSELKTHSNRAVIIIAPVLGVAFLAAVCVLLACRKFKKRPDTSAELMFKRMEALTSGNETASNQVKLKELPLFEFQVLATATDSFSLRNKLGQGGFGPVYKGKLSEGQEVAVKRLSQASGQGLEELMNEVVVISKLQHRNLVKLLGCCIEGEERLLVYEYMPNKSLDAYLFDPLKQKILDWKTRFNIMEGICRGLLYLHRDSRLKIIHRDLKVSNILLDDNLNPKISDFGLARVFRANEDEANTRRVVGTYGYMSPEYAMEGLISEKSDVFSLGVIFLEILSGRKNSHKEENNLNLLAYAWKLWNEGEAASLADPTIFDKSFEKEITRCVQIGLLCAQEAANDRPNVSTVIWMLTTENTNLQEPKQRALIARRGSCDQGSLSTNDLSLTAVTGR

SEQ ID NO: 29 - DNA, 2496 bp, Brassica napus
atggtgcttcttctattcaacacacgtcgtcgtttcgttattgttcttctacttgtaacactctcttgcttctcctttaggctctgtttcggccaagacagaatcacctcctttactactccaatcaaggacacagatacccttctctgcaaaagtggtgttttcaggtttggtttcttcactcctgtaaattccactactcggttgcgttacgtcgggatttggtacgacaagattccaaaacaaactgtggtttgggtggctaacaaagacactcccatcaacgacgcttccggtgttgtttccatctccgacgacggaaacctcgtgattacagatggtcgaaaccgccttctatggtcgaccaacgtcacagtaccacccgtggctccaaatgctacttgggttcagctgatggatactgggaaccttgcgttacaagataaccgaaacaacggagagattctctgggagagtttcaagcatccttataactctttcttgccaacaatgtctcttgggaccaacaagagaaccggagagaatctgaagcttacttcttggagaagctatcaagatccttcaacagggaactatacagctggtcttgctcctttaacgtttcccgagctcacgtttcctgagcttctgatttggaagaacaatgttccaatctggcgtagtggaccgtggaacggccaggttttcatcggtttaccggacgtggattctcttttgtttcttgatgggtttaatcttatcaatgataagcaaggaacattttcaatgtcatttgctaatgattctttcatgtatcactttaacttggatcctgatggagttatatatcagagagattggagtacttctttgagagattggaggatcggtgcaatgtttccatctacatattgtgatgcatacggtatatgtggtccatacggaagctgcagttcccgggaagatccgccttgtgaatgtgttaaagggtttgtgccgaggaacagcacagagtggaaagcaaggaattggagtaatggatgtgtgagaaaaggtcaattgcggtgcgagaggcagagcaatggaggaggaaaaggagatgtgtttgtgagacttcagaagatgaaagtaccagtcaatgcggtacaatctgatgctaatgagcaagattgtcctaaacagtgtaaggataactgttcttgcattgcttatgcttttgatcggggaatcggatgcatgctttggagtggtaacttagttgatatgcaatcatcattgagaactggcattgatctttatattcgacttccgcattctgaactcaaaacatatagcaatcgagcagttatcatcattgcacccgtgctaggcgttgcgttccttgctgctgtctgcgttcttttagcatgcaggaaattcaaaaaacgtccagatacaagtgcagaattaatgtttaagagaatggaagcactcacaagtggtaatgagactgcttctaaccaagtgaagctcaaggagcttccactctttgagtttcaagtgttagccacagcaactggtagcttctctctaagaaacaagctcggacaaggtggatttggtcctgtttacaagggaaaattgtcagaagggcaagaaattgcagtgaagaggctctcacaggcatcaggacaaggacttgaggagcttatgaacgaagtggttgtgatttccaagttgcaacatcggaatctagtgaagttacttggctgttgcattgaaggtgtagaaagactgttagtctacgaatatatgcctaataaaagcttggatgcctatctatttgacccattgaagcaaaagattcttgactggaagacacggttcaacataatggaagggatttgcagaggtctcttgtatcttcacagagattcaagactaaagatcatacacagagatctaaaagtcagcaacattttgttagatgacaacctgaatcccaaaatatctgatttcgggcttgcaagagtttttcgagcaaatgaagatgaagctaacacaagaagggttgttggaacatacggctatatgtcacctgaatatgcaatggaaggtttaatttcagaaaaatcagacgtttttagtttgggggttatatttttagagatcctaagtggcagaaaaaactctcacaaggaagagactaatctaaaccttttagcttatgcgtggaagctgtggaacgaaggtgaggctgcttctctagctgatccaaccatctttgataagtctttcgagaaagagataacgagatgtgttcagattggtttgttgtgtgcgcaagaaactgcaaacgatagaccaaatgtttcaaccgtgatatggatgctaactaccgaaaacacgaacctccaagagccgaagcagcgtgcgttaatagcaagaagaggatcttgtgaccagggtagtctctctatcaatgatttgagcctcacagctgtaacagggcgttag

SEQ ID NO: 30 - protein, 831 aa, Brassica napus
MVLLLFNTRRRFVIVLLLVTLSCFSFRLCFGQDRITSFTTPIKDTDTLLCKSGVFRFGFFTPVNSTTRLRYVGIWYDKIPKQTVVWVANKDTPINDASGVVSISDDGNLVITDGRNRLLWSTNVTVPPVAPNATWVQLMDTGNLALQDNRNNGEILWESFKHPYNSFLPTMSLGTNKRTGENLKLTSWRSYQDPSTGNYTAGLAPLTFPELTFPELLIWKNNVPIWRSGPWNGQVFIGLPDVDSLLFLDGFNLINDKQGTFSMSFANDSFMYHFNLDPDGVIYQRDWSTSLRDWRIGAMFPSTYCDAYGICGPYGSCSSREDPPCECVKGFVPRNSTEWKARNWSNGCVRKGQLRCERQSNGGGKGDVFVRLQKMKVPVNAVQSDANEQDCPKQCKDNCSCIAYAFDRGIGCMLWSGNLVDMQSSLRTGIDLYIRLPHSELKTYSNRAVIIIAPVLGVAFLAAVCVLLACRKFKKRPDTSAELMFKRMEALTSGNETASNQVKLKELPLFEFQVLATATGSFSLRNKLGQGGFGPVYKGKLSEGQEIAVKRLSQASGQGLEELMNEVVVISKLQHRNLVKLLGCCIEGVERLLVYEYMPNKSLDAYLFDPLKQKILDWKTRFNIMEGICRGLLYLHRDSRLKIIHRDLKVSNILLDDNLNPKISDFGLARVFRANEDEANTRRVVGTYGYMSPEYAMEGLISEKSDVFSLGVIFLEILSGRKNSHKEETNLNLLAYAWKLWNEGEAASLADPTIFDKSFEKEITRCVQIGLLCAQETANDRPNVSTVIWMLTTENTNLQEPKQRALIARRGSCDQGSLSINDLSLTAVTGR

SEQ ID NO: 31 - DNA, 678 bp, Brassica napus
atggctgtagaatccgactactcttttcttgaatcgataagacaatacttactagaagaatcggagttacgactcactgagtcaatggtggcgcaatctggtacgacggttcacagcgtgagaccggtgtacggacgaaactcgagcttcagcaagctgtatccttgtttctctgagagctggggagacttgccgttgaaagagaacgactcggaggacatgttagtctacggaatcctcaacgacgcttttcacggcggttgggaaccgtcttcatcttcctcagacgaagatcggagctctttcgcgacgaagacgagctctgttcaagcgccggtgaaagggaagcattacagaggagttagggagaggccgtgggggaaattcgcggcggagataagggatccggcgaagaacggagctagagtttggttggggacgtttgagacggcggaggacgcggcgttggcctacgacaaagcggctttcaggatgcgtggttcccgcgctttgttgaatttcccgttgagagttaactcgggagaacccgacccggttcggataaagtccaagagaggctcttcatccgaaaacagagctccaaagcggaggagaacggtggcttccgccggacaaggaacagacgtgggactaaaagtcaagtgtgagattgtttag

SEQ ID NO: 32 - protein, 225 aa, Brassica napus
MAVESDYSFLESIRQYLLEESELRLTESMVAQSGTTVHSVRPVYGRNSSFSKLYPCFSESWGDLPLKENDSEDMLVYGILNDAFHGGWEPSSSSSDEDRSSFATKTSSVQAPVKGKHYRGVRERPWGKFAAEIRDPAKNGARVWLGTFETAEDAALAYDKAAFRMRGSRALLNFPLRVNSGEPDPVRIKSKRGSSSENRAPKRRRTVASAGQGTDVGLKVKCEIV

SEQ ID NO: 33 - DNA, 693 bp, Brassica napus
atgatgtacggacagagcgaggtagaatccgactacgctttgttggagtcgatacgacgtcacttgctaggaggagaggccgagttgtggttcgctgagtcaataccgagttcttgtttcacagagagctggggagacttgccgttgaaagagaacgattccgaagatatgttagtctacggtctccttaacgacccctatgacacgtcatcgccgtcgtccgacttgagttgtatcaccgacttcttagacttagaaacgtcctcgaagcgccctagcgatcctccggtggttaaagccgaaccggcggagagcttcgcagcggcgacggtggagaaacagaaggcagcgacggcgaaggggaagcattacagaggggtgagacagaggccgtgggggaaattcgcggcggagatacgagatccggcgaaaaacggagcgagggtttggttagggacgtttgagacggcggaggacgcggcgtttgcgtacgatagagctgcttttaggatgcgtggttcccgcgctttgttgaatttcccgttgagagttaattccggtgagcctgatccggtgaggatcacgtcaaagaggtcttatacttcttcttcctcagaaaacgggaagctgaaacggaggagaaaaacagagaacgtaccgtccgagttccaggtgaaatgcgaggttgtgtaa

SEQ ID NO: 34 - protein, 230 aa, Brassica napus
MMYGQSEVESDYALLESIRRHLLGGEAELWFAESIPSSCFTESWGDLPLKENDSEDMLVYGLLNDPYDTSSPSSDLSCITDFLDLETSSKRPSDPPVVKAEPAESFAAATVEKQKAATAKGKHYRGVRQRPWGKFAAEIRDPAKNGARVWLGTFETAEDAAFAYDRAAFRMRGSRALLNFPLRVNSGEPDPVRITSKRSYTSSSSENGKLKRRRKTENVPSEFQVKCEVV

SEQ ID NO: 35 - DNA, 690 bp, Brassica napus
atggagtatcaaactagcttcttctacgaaaaccaagctcgagagttctcaccggagaactcttgttcaacaaactcatggagctcacaagaatcattcttgtgggaagagagtttcttacatgatcaatcatttagcccttcctactactctgacgacttcttagcatttggatcatcactcatcaaggaagaagaagaaggagaagacaccgtggcggccttggaggaggaggagaggtcatacagaggagtgaggacgaggccgtgggggaaatacgcagccgagataagagactcaacgaggaaagggataagagtgtggctcgggacgttcgacactgcggaagaggctgcccttgcttatgatcaggcagctttcgctttgaaaggtgaccttgcggtactcaacttccccgtagatgttgttagggactctctccagaagatggaggatgtaaatctcgataacggagagtctccggtgatagctttgaagagaaaacattccatgagaaaccgacccagaggaaagaagaagtctcctccatcttcctcttcctcttcgtcttcatcttcaagaagtagaatacaaagtgttgctatgaagcaagaaagtactacaacacttgtagtttttgaggatttgggtgctgagtacttagaagaacttatgagatcgtgttcatga

SEQ ID NO: 36 - protein, 229 aa, Brassica napus
MEYQTSFFYENQAREFSPENSCSTNSWSSQESFLWEESFLHDQSFSPSYYSDDFLAFGSSLIKEEEEGEDTVAALEEEERSYRGVRTRPWGKYAAEIRDSTRKGIRVWLGTFDTAEEAALAYDQAAFALKGDLAVLNFPVDVVRDSLQKMEDVNLDNGESPVIALKRKHSMRNRPRGKKKSPPSSSSSSSSSSRSRIQSVAMKQESTTTLVVFEDLGAEYLEELMRSCS

SEQ ID NO: 37 - DNA, 519 bp, Brassica napus
atgacatccctttattcgaaagtccatttgaaggcccacgtcatggagctaatgcttcaacgtctcaatgatgatgtcatcatcaatctccaccatccattttatttggcggttgacgacatcgaccgctcaaagtccacaccgacgtcctataagaagactaacatgagctcggaaaagaaagtggaacgccgtgaagaagataacggtgacggtcacatagggaattcgtcgatggaagttgtacgtacggtgacggaagaagaggtggatgagttcttcaagatattacggagagtacacgtggcgaccaggacggttacgagagctaacggcggtattgaaacccgagagttaacgtctaagaagaggaaacggagtcagaggcttggattgaggagctcattggatagtaacggcgttcgagacggagaattagaaggaatagatcgggtcgggttacggaactcgggtttggatcttaactgtaaaccggaacccgacgcagttagcttgtaa

SEQ ID NO: 38 - protein, 172 aa, Brassica napus
MTSLYSKVHLKAHVMELMLQRLNDDVIINLHHPFYLAVDDIDRSKSTPTSYKKTNMSSEKKVERREEDNGDGHIGNSSMEVVRTVTEEEVDEFFKILRRVHVATRTVTRANGGIETRELTSKKRKRSQRLGLRSSLDSNGVRDGELEGIDRVGLRNSGLDLNCKPEPDAVSL

SEQ ID NO: 39 - DNA, 2079 bp, Brassica napus
atgtcctacgcatccctcagcgtcaaggacctgaccagccttgtatcaagatccggaaccggttcgacttcttctcccaagcttccgggtcagaaccgacccgttaaggtcatcccgctccagcaccctgacacctccgatgatgctcgtcctccatcgatccctttcgacgatatcttatccggatggagggcgaagatcaagcggatgagcctcgtggattgggttgagactctgtttccttgcttcagatggattcgaacttataaatggagtgagtattttaagctcgatctcatggctggtatcaccgttggtataatgcttgttcctcagcaggcaatgtcgtatgcaaaattagctggccttccaccaatatacggtctatactcatcgtttgttccgatatttgtctatgccatattcggctcatctcgtcagcttgctattggacctgtagcattggtgtcacttcttgtgtccaatgctttgggaggaatcgctgattcgtctgaggaagagttacacattgaacttgctattttgctggcgcttttggttggaatattggagtgcatcatggggctcttaaggcttggatggcttattcgtttcattagtcactcggtcatatctggatttacaagtgcttcggccattgtgattggattatctcaagtgaaatatttcttggggtataacattgctaggagcagcaagattgtgccactagttgagagcattatagctggagctgataagtttcaatggccacctttcttgatgggatctctcattctagtaatccttcaagtgatgaagcatgtggggaaagcaaagaaggaacttcagttcttacgagcagcagcgccactcacagggatagttctcggtacaaccattgcaaaagtgttccatccaccttccatctctctggtgggagaaatacctcaaggacttccaactttttctttcccaagaagctttgatcatgcaaaaacattgcttccaacatcagctctcattaccggtgtcgccatcttggaatctgtgggtatagccaaagcacttgcagcaaaaaacagatatgagttggattcaaattcagagttgtttggtcttggtgttgcaaatatattggggtcactattttccgcatatccatctacaggatccttttctaggtcagctgtgagtaatgaaagtgaagctaagaccggcctatcaggacttataacaggaatcatcattggatgttctctactgttcttgacaccggttttcaaatacataccacagtgtgctttggcagcaattgtgatctctgctgttagtggcttggtggactatgatgaagctatcttcctatggcgtgtggacaagagggattttacactttggaccatcactagcaccacaactttgttcttcggaatagagattggtgtccttgttggtgttggtttttcactagcattcgttatccacgagtctgcaaacccacatattgctgtcttggggcgtcttccaggcaccacagtgtacagaaacgtgaagcagtatccagaggcttacacatacaacgggattgtgattgtcagaatcgatgctcctatttactttgccaacataagctacattaaagacagacttcgagaatatgaagtagctgttgacaaatacacaaccaagggaccagaggtggagagaatctcttttgttatcctggaaatgtcacctgttactcacatagactcaagcgcggtggaagcattgaaagaactgtatcaggagtacaaagcaagggacattcagctagcgatttcaaatccaaacaaagacgttcacatgacaatagcgagatcaggaatggtggagcttgttggaaaagaatggtactttgttagagtacacgacgcagtacaagtgtgtcttaattgcgtacaaagctctagcttggaagacaagaaaccaagtttcttgagaaggtttagcaacaataacggttcatcatcatacaatgatctccaatcctacaacactttgttgaaggagccattgttgccagtggagaaataa

SEQ ID NO: 40 - protein, 692 aa, Brassica napus
MSYASLSVKDLTSLVSRSGTGSTSSPKLPGQNRPVKVIPLQHPDTSDDARPPSIPFDDILSGWRAKIKRMSLVDWVETLFPCFRWIRTYKWSEYFKLDLMAGITVGIMLVPQQAMSYAKLAGLPPIYGLYSSFVPIFVYAIFGSSRQLAIGPVALVSLLVSNALGGIADSSEEELHIELAILLALLVGILECIMGLLRLGWLIRFISHSVISGFTSASAIVIGLSQVKYFLGYNIARSSKIVPLVESIIAGADKFQWPPFLMGSLILVILQVMKHVGKAKKELQFLRAAAPLTGIVLGTTIAKVFHPPSISLVGEIPQGLPTFSFPRSFDHAKTLLPTSALITGVAILESVGIAKALAAKNRYELDSNSELFGLGVANILGSLFSAYPSTGSFSRSAVSNESEAKTGLSGLITGIIIGCSLLFLTPVFKYIPQCALAAIVISAVSGLVDYDEAIFLWRVDKRDFTLWTITSTTTLFFGIEIGVLVGVGFSLAFVIHESANPHIAVLGRLPGTTVYRNVKQYPEAYTYNGIVIVRIDAPIYFANISYIKDRLREYEVAVDKYTTKGPEVERISFVILEMSPVTHIDSSAVEALKELYQEYKARDIQLAISNPNKDVHMTIARSGMVELVGKEWYFVRVHDAVQVCLNCVQSSSLEDKKPSFLRRFSNNNGSSSYNDLQSYNTLLKEPLLPVEK

SEQ ID NO: 41 - DNA, 885 bp, Brassica napus
atggaaggattagctatcagagcatcgcgaccgtccatcatctgctctcttccaggtctcgacggcggttctcagcgactgcctctaagtgacggtttcctaaggctcccaacgtcatcatatgctgcagataagccaaaactagtcgcgaagtctgcttctttacatccgatctccgccgttaatgtctctgctcaagcttccctcaccgccgattttcccgccctttcagagacgaatgtgaaagaagagagaatcaacggagacaagaataagccagagaacatcgtgtggcacgagagttccatttgcagatgcgaccgacaacagcttcttcaacagaagggttgtgtcatttggatcactggtctcagcggctcagggaaaagcactgttgcttgtgccttaagtaaagctctgttcgaaagaggcaaacttacttacacacttgacggagacaatgtacgtcatggtcttaaccgggacctcactttcaaagctgaggatcgtaccgaaaacatacgcaggatcggtgaggtggcaaagctgtttgctgacgttggcgtcatttgtatagcaagtttgatttctccttaccggagagacagagatgagtgccggtcgttgttacccgagggagatttcgtggaggttttcatggatgttcctctgtctgtgtgcgagtcaagagatccaaaggggttgtacaagctcgcacgtgccggcaaaatcaaaggctttactggaatcgatgatccttacgaggcgccactgaactgcgaggtcgttctgaaacacaccggagatgacgattcttgttcaccacgtcagatggctgaacatatcatctcctacttgcaaaacaaagggtaccttgagggctaa

SEQ ID NO: 42 - protein, 294 aa, Brassica napus
MEGLAIRASRPSIICSLPGLDGGSQRLPLSDGFLRLPTSSYAADKPKLVAKSASLHPISAVNVSAQASLTADFPALSETNVKEERINGDKNKPENIVWHESSICRCDRQQLLQQKGCVIWITGLSGSGKSTVACALSKALFERGKLTYTLDGDNVRHGLNRDLTFKAEDRTENIRRIGEVAKLFADVGVICIASLISPYRRDRDECRSLLPEGDFVEVFMDVPLSVCESRDPKGLYKLARAGKIKGFTGIDDPYEAPLNCEVVLKHTGDDDSCSPRQMAEHIISYLQNKGYLEG

SEQ ID NO: 43 - DNA, 822 bp, Brassica napus
atggcggctactacattgaacaactcttcttgtcttctccaacccaagtccagctccaccgctcgccttaacccttcttctctcctcaagcccagagtttcgttttcggggaagagtcgtggacatgtcgttacgaaagcttcgattgaaatggcgcattcgaactcgacacctgccgctgttgtcaactcctcgagtaagcacaagggtcccatcatcgtgatcgataattacgacagctttacttacaatctctgtcagtatatgggagagctaggatgccattttgaagtttaccgcaatgatgaacttactgtagaggagctcaaaagtaaaaatccaagaggggtgctgatttctcctgggcctggtaccccacaagactctgggatctccttacaaactgtgttggaactcggaccacgtgttcctttgtttggagtatgtatgggtttgcagtgtataggagaagcttttggaggaaagatcgtgaggtcaccatatggtgtaatgcatgggaaaagctccatggttcactatgatgagaaaggagaagaaggcttgttctctggtttatccaacccttttcttgtaggtagatatcacagcctagtgatcgagaaagatacctttcccagtgatgaactcgaggttactgcgtggacagaagatggtttggttatggcggcccgacacaggaagcacaagcatatacagggagtccagtttcatccggagagtatcataacaactgaaggcaagacaattgttcgcaacttcatcaaacttgtagagaaaagagaggctgaaaagttgacttag

SEQ ID NO: 44 - protein, 273 aa, Brassica napus
MAATTLNNSSCLLQPKSSSTARLNPSSLLKPRVSFSGKSRGHVVTKASIEMAHSNSTPAAVVNSSSKHKGPIIVIDNYDSFTYNLCQYMGELGCHFEVYRNDELTVEELKSKNPRGVLISPGPGTPQDSGISLQTVLELGPRVPLFGVCMGLQCIGEAFGGKIVRSPYGVMHGKSSMVHYDEKGEEGLFSGLSNPFLVGRYHSLVIEKDTFPSDELEVTAWTEDGLVMAARHRKHKHIQGVQFHPESIITTEGKTIVRNFIKLVEKREAEKLT

SEQ ID NO: 45 - DNA, 1623 bp, Brassica napus
atgaacacctttacctcaaactcttcggatctcacttccactacaacgcaaacgtctccgttcagcaacatgtatctcctcacaacgctccaggcctttgcggctataaccttggtgatgcttctcaagaaagtcttcacgacggataaaaagaaattgtctctcccgccgggtcccaccggatggccgatcatcggaatggttccaacgatgctaaagagccgtcccgttttccggtggctccacagcatcatgaagcagctaaacaccgagatagcctgcgtgaggctaggaaacactcacgtgatcaccgtcacatgcccgaagatagcacgtgagatactcaagcaacaagacgctctcttcgcctcgagacccatgacttacgcacagaatgtcctctctaacggatacaaaacatgcgtgatcactcccttcggtgaacaattcaagaaaatgaggaaagtcgtgatgactgaactcgtttgtcccgcgaggcacaggtggcttcaccagaagagagctgaagagaacgaccatttaaccgcttgggtatacaacttggtcaagaactctggctcagtcgattttcggtttgtcacgaggcattactgtggaaatgctatcaagaagcttatgttcgggacaagaacgttctctgaaaacaccgcacctgacggtggaccaaccgctgaggatatcgagcatatggaagctatgttcgaagcattagggtttactttctccttttgtatctctgattatctacctatgctcactggacttgatcttaacggccacgagaagatcatgagggattcgagtgctattatggacaagtatcacgatcctatcgtcgatgcaaggatcaagatgtggagagaaggaaagagaactcaaatcgaggattttctagacatttttatttctatcaaggatgaacaaggcaacccattgcttaccgccgatgaaatcaaacccaccattaaggaacttgtaatggcggcgccagacaatccatcaaacgctgtcgagtgggccatggcggagatggtgaacaaaccggagatactccataaagcaatggaagaaatagacagagttgtcggaaaagaaagacttgtccaagaatccgacattccaaaattaaattacgtcaaagctatcctccgtgaagccttccgcctccatcccgtagcggcctttaacctcccacacgtggcactttccgacgcaaccgtcgccgggtatcacatccctaaaggaagtcaagtccttctcagtcgatatgggctgggccgtaacccgaaagtttgggctgaccccttgagctttaaaccggagagacatctcaacgaatgctcggaagttactttgacggagaacgatctccggtttatctcgtttagtaccgggaaaagaggttgtgctgctccggctttaggtacggcgttgaccacgatgatgctcgcgagacttcttcaaggtttcacttggaagctgccggagaatgagacacgcgttgagctgatggagtctagccatgatatgtttttggctaaaccattggttatggtcggtgagttgagactcccagagcatctttacccgacggtgaagtaa

SEQ ID NO: 46 - protein, 540 aa, Brassica napus
MNTFTSNSSDLTSTTTQTSPFSNMYLLTTLQAFAAITLVMLLKKVFTTDKKKLSLPPGPTGWPIIGMVPTMLKSRPVFRWLHSIMKQLNTEIACVRLGNTHVITVTCPKIAREILKQQDALFASRPMTYAQNVLSNGYKTCVITPFGEQFKKMRKVVMTELVCPARHRWLHQKRAEENDHLTAWVYNLVKNSGSVDFRFVTRHYCGNAIKKLMFGTRTFSENTAPDGGPTAEDIEHMEAMFEALGFTFSFCISDYLPMLTGLDLNGHEKIMRDSSAIMDKYHDPIVDARIKMWREGKRTQIEDFLDIFISIKDEQGNPLLTADEIKPTIKELVMAAPDNPSNAVEWAMAEMVNKPEILHKAMEEIDRVVGKERLVQESDIPKLNYVKAILREAFRLHPVAAFNLPHVALSDATVAGYHIPKGSQVLLSRYGLGRNPKVWADPLSFKPERHLNECSEVTLTENDLRFISFSTGKRGCAAPALGTALTTMMLARLLQGFTWKLPENETRVELMESSHDMFLAKPLVMVGELRLPEHLYPTVK

SEQ ID NO: 47 - DNA, 1623 bp, Brassica napus
atgaacacttttacctcaaactcttcggatctcacttccactacaacgcaaacgtctccgttcagcaacatgtatctcctcacaacgctccaggcctttgtggctatatccttggtgatgcttctcaagaaagtcttcaccacggacaaaaagaaattgtctctcccgccgggtcccaccggatggccgatcatcggaatggtcccaacgatgctaaagagccgtcccgttttccggtggctccacagcatcatgaagcagctaaacaccgagatagcctgcgtgaggctaggaaacactcacgtgatcaccgtcacatgccctaagatagcacgtgagatactcaagcaacaagacgctctcttcgcctcgagacctatgacatacgcacagaatgtcctctctaacggatacaaaacatgcgtgatcactcctttcggtgaacaattcaagaaaatgaggaaagtcgtgatgactgaactcgtttgtcccgcgaggcacaggtggcttcaccagaagagagcagaagagaacgaccatttaaccgcttgggtatacaacctggtcaagaactcgggctcggtcgattttcggttcgtcacgaggcattactgtggaaatgctatcaagaagcttatgttcgggacaagaacgttctctgaaaacactgcaccggacggtggaccgaccgctgaggatatcgagcatatggaagctatgttcgaagcattagggttcactttctccttttgtatctctgattatctacctatgctcacgggacttgatcttaacggccacgagaagatcatgagagattcgagtgctattatggacaagtatcacgatcctatcgtcgatgcacggatcaagatgtggagagaaggcaagagaactcaaatcgaggattttctagacatttttatttctatcaaggatgaacaaggaaacccattgcttaccgccgatgaaattaaacccaccattaaggagcttgtaatggcggcgccagacaatccatcaaacgccgtagaatgggccatggcggagatggtgaataaaccggagattctccgcaaagcaatggaagaaatcgacagagttgtcggaaaagaaagacttgtccaagaatccgacatcccaaaactcaactacgtcaaagctattctccgtgaagccttccgtctccatcccgtcgccgcctttaacctcccacacgtggcactttccgacgcaaccgtcgccggatatcacatccctaaagggagccaagtccttctcagtcgatatgggctgggccgtaacccgaaagtttgggctgaccccttgagctttaaaccggagagacatctcaatgaatgctcggaagttactttgacggagaacgatctccggtttatctcgtttagtacgggtaaaagaggttgtgctgctccggctttaggtacggcgttgaccacgatgatgctcgcgagacttcttcaaggtttcacttggaagttgcctgagaatgagacacgtgttgagctgatggagtccagtcatgatatgtttttggctaaaccattggttatggtcggtgagttgagactcccagagcatctttacccgacggtgaagtaa

SEQ ID NO: 48 - protein, 540 aa, Brassica napus
MNTFTSNSSDLTSTTTQTSPFSNMYLLTTLQAFVAISLVMLLKKVFTTDKKKLSLPPGPTGWPIIGMVPTMLKSRPVFRWLHSIMKQLNTEIACVRLGNTHVITVTCPKIAREILKQQDALFASRPMTYAQNVLSNGYKTCVITPFGEQFKKMRKVVMTELVCPARHRWLHQKRAEENDHLTAWVYNLVKNSGSVDFRFVTRHYCGNAIKKLMFGTRTFSENTAPDGGPTAEDIEHMEAMFEALGFTFSFCISDYLPMLTGLDLNGHEKIMRDSSAIMDKYHDPIVDARIKMWREGKRTQIEDFLDIFISIKDEQGNPLLTADEIKPTIKELVMAAPDNPSNAVEWAMAEMVNKPEILRKAMEEIDRVVGKERLVQESDIPKLNYVKAILREAFRLHPVAAFNLPHVALSDATVAGYHIPKGSQVLLSRYGLGRNPKVWADPLSFKPERHLNECSEVTLTENDLRFISFSTGKRGCAAPALGTALTTMMLARLLQGFTWKLPENETRVELMESSHDMFLAKPLVMVGELRLPEHLYPTVK

SEQ ID NO: 49 - DNA, 1632 bp, Brassica napus
atggatactatagcatcaaactcttcggatctcacgaccaagcctagtccccaaacatctccgtttagcaacatgtatttcctcacaacacttcaagctcttgtagtcatttctctcttaatgataatcaagaaaataaagtcatcttcacataacaagaagctgcaccctctaccaccgggtcccagcgggtttccgatcgtagggatgcttccagcaatgcttaaaaaccgtccggttttccggtggcttcatagcctcatgaaagagcttaacacggagatagcttgtgtgcgtctaggaaacactcacgtcattccagtcacatgtcctaagatagcgcgtgagatattcaagcaacaagacgcactctttgcatcaagaccactcacttacgctcaaaagatactctccaacgggtacaaaacatgcgtgatcacacccttcggcgaacaattcaagaagatgaggaaagtgatcatgacggagattgtttgtccggcaaggcaccgatggctacatgataatagagctgaggaaaccgatcatctaaccgcttggctttataacatggttaaaaactctgaaccggtcgatctccgctttgttacaaggcattactgtggaaatgctattaagaggcttatgttcggaacgaggacgttctcggagaaaaccaaaaccgatggtggaccgaccatggaagatattgagcatatggaagctatgtttgaggggttagggtttacgtttgcgttttgtgtatcggactatctacccatgcttacgggattggatctaaacggacatgagaagatcatgagagaagctagtgcaattatggataaatatcatgatcctattattgatgagaagattaaaatgtggagagaaggaaagagaactcagattgaagattttctagacattttcatctctatcaaggacgcagatggccaccctttgcttaccgctgatgaaatcaaaccaaccattaaggaacttgtaatggcggctccagacaatccatcgaacgccgtggaatgggccatggcggagatgataaataaaccggagatccttcagaaagcaatagaagagatagaaagagtcgtcggcaaagaaagactcgtccaagaatccgacatacctaaacttaactatctcaaagctattatccgtgaagctttccgtcttcatcccgtcgccgcgtttaacctcccacatgtggccctttccgatacaaccgtcgctggttaccatatccctaaaggaagtcaagttttacttagccgttacggtcttggtcgtaaccctaaggtttggtctgatccactgagttttaaaccggagagacatctcaatgagtgcttggaagtgactttaaccgagaacgatctccggtttatctcgtttagtaccggaaagagaggatgtgctgctcctgcgttaggtacggccataactgtcatgatgctcgctaggcttttgcaagggtttaagtggaaactagctggaggtgagacacgtgtcgagttgatggaatcgagccatgatatgtttcttgcgaagcctttggttatggtcggagaattgaggttgtcggaggaactgtacccgatggtaaactga

SEQ ID NO: 50 - protein, 543 aa, Brassica napus
MDTIASNSSDLTTKPSPQTSPFSNMYFLTTLQALVVISLLMIIKKIKSSSHNKKLHPLPPGPSGFPIVGMLPAMLKNRPVFRWLHSLMKELNTEIACVRLGNTHVIPVTCPKIAREIFKQQDALFASRPLTYAQKILSNGYKTCVITPFGEQFKKMRKVIMTEIVCPARHRWLHDNRAEETDHLTAWLYNMVKNSEPVDLRFVTRHYCGNAIKRLMFGTRTFSEKTKTDGGPTMEDIEHMEAMFEGLGFTFAFCVSDYLPMLTGLDLNGHEKIMREASAIMDKYHDPIIDEKIKMWREGKRTQIEDFLDIFISIKDADGHPLLTADEIKPTIKELVMAAPDNPSNAVEWAMAEMINKPEILQKAIEEIERVVGKERLVQESDIPKLNYLKAIIREAFRLHPVAAFNLPHVALSDTTVAGYHIPKGSQVLLSRYGLGRNPKVWSDPLSFKPERHLNECLEVTLTENDLRFISFSTGKRGCAAPALGTAITVMMLARLLQGFKWKLAGGETRVELMESSHDMFLAKPLVMVGELRLSEELYPMVN

SEQ ID NO: 51 - DNA, 1500 bp, Brassica napus
atggatctcttcttgattattgccgcgatggtagcagtcgcagccttcttcctccttcggagctccacgaagaaatctctccggctgcctccggggccaaaaggtcttcctattattggaaacctccaccagatggagaaatttaacccccaacacttcctctttcgtctctccaagctctacggtccaattttcacaatgaaaatcggtggacgccgccttgcagtgatctcatcggctgagttagccaaggaactcctcaagacccaggacctcaatttcaccgctcgtcctctcctgaaggggcaacaaacgatgtcatatcaaggtcgtgagcttggtttcggacagtacacagcgtactatcgtgagatgaggaagatgtgtatggttaacctattcagcccaaatcgcgtcgcaagtttccgacccgttagagaagaagagtgccaacgtatgatggacaagatctacaaagccgctgatcaatcagggaccgttgatttaagtgagcttctcttgtccttcaccaactgtgtcgtctgtagacaagcttttggtaagcggtataacgagtacggaaccgagatgaagagattcataaacatcttgtacgagactcaagcccttttgggcactctgtttttctccgaccttttcccttatttcggatttcttgacaatctcactggtcttaatgcgcgtctcaagagagctttcaaggagctcgacacttaccttcaagaacttctcgatgagactcttgaccctagccgtcctaaacctgagacggagagtttcattgatcttttgatgcagatctacagagatcaacctttctccatcaaatttacccacgaaaatgtcaaggccatgatattggatattgttgtaccgggaactgacactgcggcagcagtggtagtatgggccatgacttaccttataaagtaccctgaagcaatgaagaaagctcaagatgaagttaggaatgtggtcggtgacaaaggatacgtctccgaagaagacatccccaatctcccttatctgaaggccgtcatcaaggagtcactccgtctcgaaccagtcatcccaattcttctacatagagaaaccatagcagacgcgaagataggtggctatgatattccggcgaagaccatcattcaggtgaacgcatgggcggtttctcgtgacacagccgcctggggagacaaccctaatgagttcattccagagaggttcatgaacgagcagaaaggagtggacttcaaggggcaagattttgagctcctacctttcgggtcaggcagaagaatgtgccccgctatgcatcttggagtcgcaatggtagagataccttttgctaaccttctctaccgattcgattggagcctaccgacagggattaaacctgaggacataaagatggacgtcatgaccggactcgctatgcataagaaagatcacctcgtccttgcaccaaggacgcacatttga

SEQ ID NO: 52 - protein, 499 aa, Brassica napus
MDLFLIIAAMVAVAAFFLLRSSTKKSLRLPPGPKGLPIIGNLHQMEKFNPQHFLFRLSKLYGPIFTMKIGGRRLAVISSAELAKELLKTQDLNFTARPLLKGQQTMSYQGRELGFGQYTAYYREMRKMCMVNLFSPNRVASFRPVREEECQRMMDKIYKAADQSGTVDLSELLLSFTNCVVCRQAFGKRYNEYGTEMKRFINILYETQALLGTLFFSDLFPYFGFLDNLTGLNARLKRAFKELDTYLQELLDETLDPSRPKPETESFIDLLMQIYRDQPFSIKFTHENVKAMILDIVVPGTDTAAAVVVWAMTYLIKYPEAMKKAQDEVRNVVGDKGYVSEEDIPNLPYLKAVIKESLRLEPVIPILLHRETIADAKIGGYDIPAKTIIQVNAWAVSRDTAAWGDNPNEFIPERFMNEQKGVDFKGQDFELLPFGSGRRMCPAMHLGVAMVEIPFANLLYRFDWSLPTGIKPEDIKMDVMTGLAMHKKDHLVLAPRTHI

SEQ ID NO: 53 - DNA, 1500 bp, Brassica napus
atggatctcttcttgattattgccgccatggtagcagtcgcagccttcttcctcctccggagctccacgaagaaatctctccggctgcctccggggccaaaaggtcttcctattattggaaacctccaccagatggagaaatttaacccccaacacttcctctttcgtctctccaagctctacggtccaattttcacaatgaaaatcggtggacgccgccttgcagtgatctcatcggctaagttagccaaggaactcctcaagacccaggacctcaatttcaccgctcgtcctctcctgaaggggcaacaaacgatgtcatatcaaggtcgtgagcttggtttcggacagtacacagcgtactatcgtgagatgaggaagatgtgtatggttaacctattcagcccaaatcgcgtcgcaagtttccgacccgttagagaagaagagtgccaacgtatgatggaaaagatctacaaagccgctgatcaatcagggaccgttgatttaagtgagcttctcttgtcattcaccaactgtgtcgtctgtagacaagcttttggtaagcggtataacgagtacggaaccgagatgaagagattcataaacatcttgtacgagactcaagcccttttgggcactctgtttttctccgaccttttcccttatttcggatttcttgacaatctcactggtcttagtgcgcgtctcaagagagctttcaaggagctcgacacttaccttcaagaactcctcgatgagactcttgaccctagccgccctaaacctgagacggagagtttcattgatcttttgatgcagatctacaaagatcaacctttctccatcaaatttacccacgaaaatgtcaaggccatgatattggatattgttgtaccgggaactgacactgcggcagcagtggtcgtatgggccatgacttaccttataaagtaccctgaagcaatgaagaaagctcaagatgaagtgaggaatgtggtcggtgacaaaggatacgtctccgaagaagacatccccaatctcccttatctgaaggccgtcatcaaggagtcactccgtctcgaaccagtcatcccaattcttctacatagagaaaccatagcagacgcaaagataggtggctatgatattccggcgaagaccatcattcaggtgaacgcatgggcggtttctcgtgacacagccgcctggggagacaaccctaatgagttcattccagagaggttcatgaacgagcagaaaggagtggacttcaaggggcaagattttgagctcctacctttcgggtcaggcagaagaatgtgccccgctatgcatcttggagtcgcaatggtagagataccttttgctaaccttctctaccgattcgattggagcctaccgacagggattaaacctgaggacataaagatggacgtcatgaccggactcgctatgcataagaaagatcacctcgtccttgcaccaaggacgcacatttga

SEQ ID NO: 54 - protein, 499 aa, Brassica napus
MDLFLIIAAMVAVAAFFLLRSSTKKSLRLPPGPKGLPIIGNLHQMEKFNPQHFLFRLSKLYGPIFTMKIGGRRLAVISSAKLAKELLKTQDLNFTARPLLKGQQTMSYQGRELGFGQYTAYYREMRKMCMVNLFSPNRVASFRPVREEECQRMMEKIYKAADQSGTVDLSELLLSFTNCVVCRQAFGKRYNEYGTEMKRFINILYETQALLGTLFFSDLFPYFGFLDNLTGLSARLKRAFKELDTYLQELLDETLDPSRPKPETESFIDLLMQIYKDQPFSIKFTHENVKAMILDIVVPGTDTAAAVVVWAMTYLIKYPEAMKKAQDEVRNVVGDKGYVSEEDIPNLPYLKAVIKESLRLEPVIPILLHRETIADAKIGGYDIPAKTIIQVNAWAVSRDTAAWGDNPNEFIPERFMNEQKGVDFKGQDFELLPFGSGRRMCPAMHLGVAMVEIPFANLLYRFDWSLPTGIKPEDIKMDVMTGLAMHKKDHLVLAPRTHI

SEQ ID NO: 55 - DNA, 405 bp, Brassica napus
atggccaagcttatctcgtttcatgatgtggctaaacataaatgcaagaatgattgttggattctcatccatggaaaggtttatgacgtcagcagtttcatagacgaacatcccggcggtgacaatgttctcctcgccgtcactggaaaggacgcgtcgactgattttgacgatgtgaaccatagcaatgaagcgaaagagacgatgaagaaatattatatcggtgacgttgacaagtcaactgtcccggtgacggcgaagtatattccaccgtgggagaaagaatctactgctgaaaccacaaaagacgaatctggaaacaagatgcttgtatacttggttcctctcttgattctcggcgttgctttctttctcagattctacaacaacaagcagacaacttaa

SEQ ID NO: 56 - protein, 134 aa, Brassica napus
MAKLISFHDVAKHKCKNDCWILIHGKVYDVSSFIDEHPGGDNVLLAVTGKDASTDFDDVNHSNEAKETMKKYYIGDVDKSTVPVTAKYIPPWEKESTAETTKDESGNKMLVYLVPLLILGVAFFLRFYNNKQTT

SEQ ID NO: 57 - DNA, 405 bp, Brassica napus
atggccaagcttatctcgtttcatgatgtggctaaacataagtgcaagaatgattgttggattctcatccatggaaaggtttatgacgtcagcagtttcatagacgaacatcccggcggtgacaatgttctcctcgccgtcaccggaaaggacgcgtccactgattttgacgatgtgaaccatagcaatgaagcgaaagagacgatgaagaaatattatataggtgacgttgacaagtcaactgtcccggtgacggcgaagtatattccaccgtgggagaaagaatctacagctgaaaccacaaaagacgaatctggaaacaagatgcttgtatacttggttcctctcttgattctcggcgttgctttctttctcagattctacaacaacaagcagacaacttaa

SEQ ID NO: 58 - protein, 134 aa, Brassica napus; identical to SEQ ID NO: 56

SEQ ID NO: 59 - DNA, 1113 bp, Brassica napus
atgggatacgtttcagaccctaaatccatgaatgagattaatggagatgatgagaccgagcttggtttgagggcggtgaggctagccaattacataaccttcccaatggttttcaaagccgccatcgaacttggtgtcatcgatactctctactcagctgctcgtgctgatatgaatggatccagttcattcctcaaaccgtctgagatagctactcggcttcctacaacgcctagtaatcctgaagcacctgctttgttggaccgtatgcttcgtttactcgctagttactcaatggtcaaatgccaaatcctagatggtgagagggtttacaaagctgaacccatttgcaagtatttcttgagatacaatattgaagaaataggaacacttgcttctcaattcattcttgaactcgacagtgtcttcctcaatacatgggcacaactgaaagatgtggtgctagaaggaggagatgcatttgctcgtgccaacggtgggttgaagctctttgattacatgggaacggatgagagactaagcaaactctttaaccggactggattcagcgttggggttttgcagaagtttctagaagtttacaaaggtttcgaaggagttaatgtgttggttgatgtaggaggaggagttggaaacacactaggctttgttacttcaaagtatccaaacattaagggtattaattttgatctaacttgtgctttgacacaagcaccttcttatcctaatgtggagcatgtggctggagatatgtttgtagaagtcccaagaggagatgctatcatcttgaaacgtatgcttcatgattggagtgatgaagactgtgcaaagattctcaagaattgctggaaagcgttaccggagaatgggaaagtgattatcatggaactagtaattccagatgaggcagagagtgcagatgtgcagtccaacattgcatttgacatggatttgttgatgctcacacaatgctctggaggaaaagagagatcaagggctgagtatgaagctatggctgcagattcgggttttgccaattgcaagtttgtatgccaagcttatcatttgtgggtcattgagttcactaaatag

SEQ ID NO: 60 - protein, 370 aa, Brassica napus
MGYVSDPKSMNEINGDDETELGLRAVRLANYITFPMVFKAAIELGVIDTLYSAARADMNGSSSFLKPSEIATRLPTTPSNPEAPALLDRMLRLLASYSMVKCQILDGERVYKAEPICKYFLRYNIEEIGTLASQFILELDSVFLNTWAQLKDVVLEGGDAFARANGGLKLFDYMGTDERLSKLFNRTGFSVGVLQKFLEVYKGFEGVNVLVDVGGGVGNTLGFVTSKYPNIKGINFDLTCALTQAPSYPNVEHVAGDMFVEVPRGDAIILKRMLHDWSDEDCAKILKNCWKALPENGKVIIMELVIPDEAESADVQSNIAFDMDLLMLTQCSGGKERSRAEYEAMAADSGFANCKFVCQAYHLWVIEFTK

SEQ ID NO: 61 - DNA, 1788 bp, Brassica napus
atgggatcggaacatggtgacgatgggcttcgtcggcgaggctgttcatgcacaaaagatgattttctcccggaggaatcgttcaaaagcatggggaactactttaaagcccttaaagaaacaccgagtcggttcatagaccgtgtaatgactcggtctgacgactcggcggagattcatgacatgaaggcacgtagtggtaacgagatgaagaaaacgttgacgtggtgggatcttatgtggtttggcgtcggtgcagttatcggctcgggaatattcgttctcaccggacagcaagcaaagggtgagtccggtccagccgttgtgttgtccttcgttgtatcaggtgtctccgccatgctctccgtgttttgttacaccgagttcgccgttgagattcctgtcgcaggcggttctttcgcctacttgagagtggagcttggcgacttcatggctttcatcgccgctggaaatataatcctcgaatacgtagtcggtggagcagctgtggctcggtcatggacctcttacttcgccactctcttaaaccacaaacctgaagacttccgcataatagcccacagtctcaacgaagactatagccacttagacccaatagccgtcggcgtttgcgcgataatttgcgttttagccgttgttggcacaaaaggctcatcagttttcaactacatcgcttctatgatccatatggtcgtcattgcgttcgtgatcatcgctggtttaactagagctgacctcaaaaactattcggacttcgctccctttggagttagaggagtgtttaaatccgcctcggttcttttctttgcgtatattggattcgatgccgtgtcaacaatggctgaggagacaaagaaccctgggagagatatccccattggtcttgtcggctcaatggtggttaccacggtttgttattgtctcatggcggttacgttgtgtctcatgcaaccgtatagtcagattgatcctgacgcgccgttttctgtcgcgttctctgcggttggatgggattgggctaagtacattgttgcgtttggtgctcttaaaggtatgacaaccgttttgttagttggagccataggtcaagctaggtacatgacgcacatagcccgtgcccacatgatgccaccatggctagctcatgtcaacgcaaagacaggaacaccgatcaacgcgacagtggtcatgcttgccgcgacagctctcatcgcattcttcacaaaacttgaaatattagccgatcttttatcggtttccacacttttcatcttcatgttcgtcgcggtggctcttcttgtgaggagatattacgtcaccggagaaacatcttcacgtgacctgaacaagttcttaatgttcttaggtttgattcttgcatcctcgatcgcgactgctgtgtattgggctatggaaagagacggttggattgtatatgccgttacggtccctatttggttcttgtctaccgtggggatgaagtttcttgtaccgcaagctagggctccgaagctatggggtgttcctttggttccgtggttgccttccgcttcgatagctattaatatatttcttcttggttcgatcgatgaaaaatcgttcgttaggtttgggatatggactggtgttcttcttgtctactacttcttgtttgggttgcacgcaacttacgatacggcgaaggcgacgctaaaggagaagtcggcgttgaagaatgctgaggaaggtagtgttgctgcagataaatctggtcgtgcagctttatag

SEQ ID NO: 62 - protein, 595 aa, Brassica napus
MGSEHGDDGLRRRGCSCTKDDFLPEESFKSMGNYFKALKETPSRFIDRVMTRSDDSAEIHDMKARSGNEMKKTLTWWDLMWFGVGAVIGSGIFVLTGQQAKGESGPAVVLSFVVSGVSAMLSVFCYTEFAVEIPVAGGSFAYLRVELGDFMAFIAAGNIILEYVVGGAAVARSWTSYFATLLNHKPEDFRIIAHSLNEDYSHLDPIAVGVCAIICVLAVVGTKGSSVFNYIASMIHMVVIAFVIIAGLTRADLKNYSDFAPFGVRGVFKSASVLFFAYIGFDAVSTMAEETKNPGRDIPIGLVGSMVVTTVCYCLMAVTLCLMQPYSQIDPDAPFSVAFSAVGWDWAKYIVAFGALKGMTTVLLVGAIGQARYMTHIARAHMMPPWLAHVNAKTGTPINATVVMLAATALIAFFTKLEILADLLSVSTLFIFMFVAVALLVRRYYVTGETSSRDLNKFLMFLGLILASSIATAVYWAMERDGWIVYAVTVPIWFLSTVGMKFLVPQARAPKLWGVPLVPWLPSASIAINIFLLGSIDEKSFVRFGIWTGVLLVYYFLFGLHATYDTAKATLKEKSALKNAEEGSVAADKSGRAAL

SEQ ID NO: 63 - DNA, 1122 bp, Brassica napus
atgaaagtgaaggttggagagtttgtgccgtttgtggcaatggtgataatggaggcatgcacgattgctcttacgataatggccaagacggcactaacgggagggatgagtccttttgttttcgttgtttacacaaacgctttgggatctattcttcttcttcccttttctttcttcttccatagaaaggacagaactaaagaatctatcttttcttggccactcttcgttcgtgttttctttctaggtttcaccgggatatttctgttccaaaatttggcatttgtgggactaagcttcagctcacccatagtagtatgtgcaatgggattactcattccttcattctccttcttgctcaatcttatcctcggaaggagcaagttggattggagaaacacgagcacgagggctagagttatgggaacaataatctcattaagcggagcattcagtgaagaattgtacaaaggtccttttataagaccagcttcgtctgcttcccctactcgtcttctaaaatcaatccctaaactattggtctactacaacatccctgataattggttcctcggctgtatctttctggcggccgctgttttttctgtctccctattcaatgttattcagacagggacggtcaaaaagtatccacacgtaatgaaagtggcttcgttttacagcatagtcgggacgatccaatgcctaatattctcgttgtttatggaaagagacctaagtgcatggaagatcgagcctaactacgatctttgcctcattattgccacgggaattttcggaagtgtaatacggacaagcgtacaagtaaagtgtacccaaatgaaaggaccatattacgtgccattattcaaaccctttggcatattttgggcaacactctttggtaccagcttcttcgtcaacagtcttcactacggcagtgtattaggagcagtcataggtggcgttggatattacacagtttcttggggacaattgagggaaaccgaagaaaaacaaaattcaaaagatgaaagaaaacccatcaaaactattcatcatcacgaagatgatgaatacaaaattccattgcttattaatcaggaagaaagtcctgtgtga

SEQ ID NO: 64 - protein, 373 aa, Brassica napus
MKVKVGEFVPFVAMVIMEACTIALTIMAKTALTGGMSPFVFVVYTNALGSILLLPFSFFFHRKDRTKESIFSWPLFVRVFFLGFTGIFLFQNLAFVGLSFSSPIVVCAMGLLIPSFSFLLNLILGRSKLDWRNTSTRARVMGTIISLSGAFSEELYKGPFIRPASSASPTRLLKSIPKLLVYYNIPDNWFLGCIFLAAAVFSVSLFNVIQTGTVKKYPHVMKVASFYSIVGTIQCLIFSLFMERDLSAWKIEPNYDLCLIIATGIFGSVIRTSVQVKCTQMKGPYYVPLFKPFGIFWATLFGTSFFVNSLHYGSVLGAVIGGVGYYTVSWGQLRETEEKQNSKDERKPIKTIHHHEDDEYKIPLLINQEESPV

SEQ ID NO: 65 - DNA, 957 bp, Brassica napus
atggattctcgaaattttctcgcgttttcgctaagtcttatcctgattttccctccaatttcgtctgcttccatcagattcatccctcgatggagtagtagtagtaatagagaaggggcagtgattaagcagaagacgagtgcttcgtctctagttattgatccaactcgtgtcactcagctctcttggactcctagggtgtttttgtacaagggacttttaacagatgaggaatgtgatcatttcatcaacttggcgaaagggaagcttgagaagtcaatggttgcggataatgattcaggtaagagcgtggagagtgaagtgcgcacaagctctggaatgtttctttccaaaagacaggatgatatagtagctaatgtcgaggctaaacttgctgcatggacttttcttcccgaggaaaatggagaatcaatgcaaatactgcactacgagaacggtcaaaaatatgaaccgcattttgacttctttcacgaccaagtaaaccaacagcttggtggtcatcggatcgccaccgtattgatgtacttgtccaacgtcaaaaaaggtggagaaacagtgtttcccatgtggaagggagcaacagctcaacccaaagacgatagctggacccaatgtgccaaacaaggttatgcagtgaaaccgatgaaaggggacgcattgctattctttaaccttcatccgaatgcaacaacagatccgagcagcttacatggaagctgtccagtggttgagggagagaaatggtcagcaaccagatggattcatgtgaagtcgttcgaaagaccggtgtccaggacaactgggtgtgtggacgagaacgagagctgcgagaaatgggcaaaggcaggggaatgtaagaagaatccagtctacatggtgggttcagaaacagaccagggatattgcagaaagagctgcaaagcttgctcatcttaa

SEQ ID NO: 66 - protein, 318 aa, Brassica napus
MDSRNFLAFSLSLILIFPPISSASIRFIPRWSSSSNREGAVIKQKTSASSLVIDPTRVTQLSWTPRVFLYKGLLTDEECDHFINLAKGKLEKSMVADNDSGKSVESEVRTSSGMFLSKRQDDIVANVEAKLAAWTFLPEENGESMQILHYENGQKYEPHFDFFHDQVNQQLGGHRIATVLMYLSNVKKGGETVFPMWKGATAQPKDDSWTQCAKQGYAVKPMKGDALLFFNLHPNATTDPSSLHGSCPVVEGEKWSATRWIHVKSFERPVSRTTGCVDENESCEKWAKAGECKKNPVYMVGSETDQGYCRKSCKACSS

SEQ ID NO: 67 - DNA, 1299 bp, Brassica napus
atggcggcgtcaacaaactctttcctcgtcggaaacaacaacactcagatcccgtctttgaaacccaaatccatatctcaatccctcctccacctcgccaaaccaaacaccttgcacctcgctggaaaaaccaaacccgcatccgtcagatgcatctcctccccgacccaaatccaagacggagccagatccgcccccaccggatcgcaagaccgcgtcttcaacttcgcggcgggccccgccactctcccggagaacgtcctcctcaaagctcaggcggatctatacaactggcgcggatcggggatgagcgtgatggagatgagccacagagggaaagagttcctctccatcatccaaaaagccgactcggatctccgccagctcctcgagattccctccgaatattccgttttgttcttgcaaggcggggccacgacccagttcgccggtttgcctctcaatctctgcaaaccggacgataccgtcgatttcgtcgtaaccggttcgtggggtgacaaagccgtgaaggaagctaagaagtattgcaagaccagcgtgatctggtctggtaagtccgacaagtacacaaaggttccgtctttcgatgcgctggagcagagtcccgacgctaagtatctgcatatatgcgctaacgagactatccatggagttgagtttaaggactatcctgtcgttagaaaccctaatgggttcttggtggctgacatgtcttctaacttctgttcgaagccggttgatgtgtctaagttcggtgtgatctacggtggtgctcagaagaacgttggtccgtccggtgtgacgatcgtgataatcaggaaagatctgatcggtaacgctcaggagatcactccagtgatgcttgactacaagattcatgatgagaacagttccttgtacaacacgcctccttgcttcgggatttacatgtgtgggctcgtgtttgaggatctgttggagcaaggcgggttgaaggaagtggagaggaagaatcagaggaaagctgatttgctttacaacgctattgaagagagcaaggggttttttaagtgtccggttgagaagtcggtgaggtcgttgatgaatgtgccttttactttggagaagtctgagttggaaggggagtttattaaggaagcggctaaggagaagatggtgcagcttaaagggcataggtctgtgggaggtatgagagcttctatttataatgctatgcctttgtctggtgttgagaagcttgttgctttcatgaaagattttcaggctaagcatgcctaa

SEQ ID NO: 68 - protein, 432 aa, Brassica napus
MAASTNSFLVGNNNTQIPSLKPKSISQSLLHLAKPNTLHLAGKTKPASVRCISSPTQIQDGARSAPTGSQDRVFNFAAGPATLPENVLLKAQADLYNWRGSGMSVMEMSHRGKEFLSIIQKADSDLRQLLEIPSEYSVLFLQGGATTQFAGLPLNLCKPDDTVDFVVTGSWGDKAVKEAKKYCKTSVIWSGKSDKYTKVPSFDALEQSPDAKYLHICANETIHGVEFKDYPVVRNPNGFLVADMSSNFCSKPVDVSKFGVIYGGAQKNVGPSGVTIVIIRKDLIGNAQEITPVMLDYKIHDENSSLYNTPPCFGIYMCGLVFEDLLEQGGLKEVERKNQRKADLLYNAIEESKGFFKCPVEKSVRSLMNVPFTLEKSELEGEFIKEAAKEKMVQLKGHRSVGGMRASIYNAMPLSGVEKLVAFMKDFQAKHA

SEQ ID NO: 69 - DNA, 675 bp, Brassica napus
atggcggctcctcatgtcacagctgttgcatctctgatcaaatcactccatcccacatggagtccatccgccatcagatcagcaattatgacaacagcgactcaaacgaacaatgacaaaggccttataacaacagaaactggtgcaacagccacgccttatgacactggatcaggagagctgagcacaacagcatcaatgcaaccaggactggtctacgagactactgctgttgactacttgaacttcctctgttactatggatacaacataagcacaatcaagaccatatcaaaatctgttccagaaaatttcacttgccctgaagattccaagctagacttgatctcaaccgttaattatccatcaattggaatctccggatacaaggggaatgagaacaagacggtgagcaggacagtgactaatgttggtggagacggtgtggctgtttacatggtcagcgtggagacaccaccagggtttagtattcaggtgacgccagagaaactgcagtttacaaaagatggtgagaagttgacataccaggtgacagtctctgctggtgatggctcacttaagaaagatgtatttggggctcttacttggtctaatgctaagtataaagtcagaagtccaattgtaattagtagcgagactagtaccacaaactga

SEQ ID NO: 70 - protein, 224 aa, Brassica napus
MAAPHVTAVASLIKSLHPTWSPSAIRSAIMTTATQTNNDKGLITTETGATATPYDTGSGELSTTASMQPGLVYETTAVDYLNFLCYYGYNISTIKTISKSVPENFTCPEDSKLDLISTVNYPSIGISGYKGNENKTVSRTVTNVGGDGVAVYMVSVETPPGFSIQVTPEKLQFTKDGEKLTYQVTVSAGDGSLKKDVFGALTWSNAKYKVRSPIVISSETSTTN

SEQ ID NO: 71 - DNA, 1038 bp, Brassica napus
atgtcttccacaaaacccatatcactccttgtctccatcactttcttcttctctctcctcctcagttcatcctccgcccagtccgtcgtcaaagcttcatactggttcccagggagtgagtatcccgtcaccgacattgactcatctctcttcactcacctcttctgcgcctttgctgacctcaacgctcaaaccaatcaagtcaccatcgcctccgcgagccagcagaatttctccactttcactcaaaccgtacaacggcgaaacccttctgtgaaaacccttttgtccatcggcggtggaaacgctagtaaaaccgcgtttgcgtccatggcaagtaaccccacttctagaaaatcgttcattgattcttcgataagacttgcgagatcgaacggcttccacggtcttgacctagactgggagtatccgagcagtgctacggagatgagcaacttcgggacgctgcttcgagaatggcgatcagcggttgcagcagaggcaagtagcagtggtagacagcgtttgcttttggcggctgcggttttttattccaacaaccactattcggtactgtacccggttcaagccgttgccgacagtctggattgggttaatcttatggcctatgatttttatggaccgggttggtctaaagtaaccggtccacctgccgctttgtatgacccttcaaacgcaggtccaagcggcgacgctggagtgagatcatggacacaagctggcctccctgcgaagaaagcagtcttgggatttccatactacggctacgcatggggcctctcaaacgccaacacggctgtgactataagcggagcggctataccagaggacgggacgacttggattgggtacgatgataatcagagtatcgtgacgaaagtgagatacgctaagcagagaggtttgcttggttacttctcttggcatgttggagccgacgataattctggtctatctcgtgcagcgacacgtgcatgggatgctgcggcaaccaccagaactatacagaagaattaa

SEQ ID NO: 72 - protein, 345 aa, Brassica napus
MSSTKPISLLVSITFFFSLLLSSSSAQSVVKASYWFPGSEYPVTDIDSSLFTHLFCAFADLNAQTNQVTIASASQQNFSTFTQTVQRRNPSVKTLLSIGGGNASKTAFASMASNPTSRKSFIDSSIRLARSNGFHGLDLDWEYPSSATEMSNFGTLLREWRSAVAAEASSSGRQRLLLAAAVFYSNNHYSVLYPVQAVADSLDWVNLMAYDFYGPGWSKVTGPPAALYDPSNAGPSGDAGVRSWTQAGLPAKKAVLGFPYYGYAWGLSNANTAVTISGAAIPEDGTTWIGYDDNQSIVTKVRYAKQRGLLGYFSWHVGADDNSGLSRAATRAWDAAATTRTIQKN

SEQ ID NO: 73 - DNA, 1350 bp, Brassica napus
atggcaaaaaccaaacatgttatgtatctcaaattatctctcacccttctcattttagctcatgttcttgccacgaccaacggtttggattctccttcgtccaataccaagcgtccctggcccttcaagaaactcaacaaaccggtggttttaatgatttcatgcgacggtttccgtttcggttaccaattcaaaaccgatacaccaaacatcgacctactcatatccgaaggaaccgaagccaaatccggtttaatcccggttttccccaccatgacattcccaaaccactactccatcgcgaccggactctacccggcttaccacggtataatcatgaactcctttaccgatccagtaaccggagataagttcaacaaaggcctagatccaaaatggtggttaggtgaaccgttgtgggtaaccgcagcaaaccaaggtcgcaaggctgtcacttacttctggccaggatctgaagtccccaaagattcttggacttgtcctaaagagttgtgtcctcattacaactcttcggttacttttgaagaaagggtcgataaggtcttgagctacttcgatcttccacagagtgatataccggacttcttgatgctgtatttcgaccaaccggacaaggaaggccatgagtatggtcctgatgatcctcgtgttacggctgcggtcgggagggtcgataaaatgatcggcagggtcattcaaggactcaagaagagggagatcttcgatgaggttaatgtgatattgcttggcgatcacggaatggtgacaaactgtgacatgaaaacaatttacattgatgatttagcagagtgggtcaagataccagcggattggatcaatgcttatagccccgtgctggcgatgaaccctaagtggggtaaagatgtcaagaatccaagcgagaagaacgcagaactcgtggctaagatgaacgagggtttgagctcaggaaaagtagagaacggtgagtttttgcaggtatacttgaaggagaagttacctaaaaggctgcactattcagagagttctcggattccgccgattgtaggaatggttggagaaggtctaattgttagacagaacagaacaggtgttcatgaatgttatggagatcatggatacgacaacaagtacttctccatgagatctatctttattggacatggtcctaggtttaggcaaggaaagaaagtgccgtccttcgaaaatgttcagatctataatgttgttgcggagattcttggactcagaccagcttccaacaatggttcttctttgttcactaggagcattctctcgtcctctggagagacaggggaagtggaatga

SEQ ID NO: 74 - protein, 449 aa, Brassica napus
MAKTKHVMYLKLSLTLLILAHVLATTNGLDSPSSNTKRPWPFKKLNKPVVLMISCDGFRFGYQFKTDTPNIDLLISEGTEAKSGLIPVFPTMTFPNHYSIATGLYPAYHGIIMNSFTDPVTGDKFNKGLDPKWWLGEPLWVTAANQGRKAVTYFWPGSEVPKDSWTCPKELCPHYNSSVTFEERVDKVLSYFDLPQSDIPDFLMLYFDQPDKEGHEYGPDDPRVTAAVGRVDKMIGRVIQGLKKREIFDEVNVILLGDHGMVTNCDMKTIYIDDLAEWVKIPADWINAYSPVLAMNPKWGKDVKNPSEKNAELVAKMNEGLSSGKVENGEFLQVYLKEKLPKRLHYSESSRIPPIVGMVGEGLIVRQNRTGVHECYGDHGYDNKYFSMRSIFIGHGPRFRQGKKVPSFENVQIYNVVAEILGLRPASNNGSSLFTRSILSSSGETGEVE

SEQ ID NO: 75 - DNA, 999 bp, Brassica napus
atggcaggtttcaagccttttggaacgttcttgatttgtgttggctccggatgcatttgcatcgacgacgctgagacggtcacggagctgctaggcggtgctaacacggatgaagtaggttggagagactacggggaagttcttggtcgtctggctcaaccgttagcggtggattcaagactgatggttgatgatggaattatcaaccgaatggaggaacgtgctgcgaacaagaggctgcgttttgatcgtcttatgaacctaaaccacgtgaagatatcaatggtatatatagaattctacaaaaaggaatgcgagaaggtaaaggcggcttactacgaccggtataagacacatatgaagtctcctatttcgccgtttgacatggatattgagaagcgcaaaatagaggtaaacgattactggaaagaattggttgaagtagtggagaagatgcctcagagcgagaagtcagtactcaagacccggtctctcttctccgggaacaattacagacggatggtcgagccgctcgacattgctgagtattacctcagtggtcggagagactatcgaaccactggaaggtctcgccactatgttatcctcgagaaatggtttaaggcagaagtgaaagaacaggttagatgggtaagtagagaccagagtaatctttcaacgtttgattcttgttactgggctgaagttgaggaagctatgattgcaaccaatactctgaaaaaacaagtggtggggaaaaatgtcttgctgcaaaaattcgcgtggtttaaagaagctatgcttggaaccaatgctctgaaaaaacaagaggtggggagagatgtcttgttgcaaaaactcgcgtggtttgaagaagacgtgcgggagatgatcagaaaaggtgaggtaccccgggagattctcttggagagaagcagtttcatgatgtggtggggagagtacacggagatcaaaaggcttacattctacactttatga

SEQ ID NO: 76 - protein, 332 aa, Brassica napus
MAGFKPFGTFLICVGSGCICIDDAETVTELLGGANTDEVGWRDYGEVLGRLAQPLAVDSRLMVDDGIINRMEERAANKRLRFDRLMNLNHVKISMVYIEFYKKECEKVKAAYYDRYKTHMKSPISPFDMDIEKRKIEVNDYWKELVEVVEKMPQSEKSVLKTRSLFSGNNYRRMVEPLDIAEYYLSGRRDYRTTGRSRHYVILEKWFKAEVKEQVRWVSRDQSNLSTFDSCYWAEVEEAMIATNTLKKQVVGKNVLLQKFAWFKEAMLGTNALKKQEVGRDVLLQKLAWFEEDVREMIRKGEVPREILLERSSFMMWWGEYTEIKRLTFYTL

SEQ ID NO: 77 - DNA, 1086 bp, Brassica napus
atggtgggtttgcgcagagcaaagacggtacttcttgtattagtggtggttatttccaacacaatagcagacggggcaccccttgatcgaggacccaaaggaagcggcgcagctgcacctcttggcgctacggttcccggaggaggcaatgcagctgcaccggttggagctgctacaacagcaccagctggagccaaaggaagccctgcagctgcaccggttggagctacagcaccagcagcagccgcaggaggccttgcagctgcaccagcaggagccgcaggagccgcaggatcacttgacgttaaagcagcaggagccaaaggagacggaacaaccgatgacactgcggcatttacggctgtatggaaaacagcatgtgaagcaccaggaccgagcacgattacagtgccaaagggtgactatttggtgaacaacatagagttcttaggtccatgcaagggtcccgtcactttcgaaatgagtggcaatatgaaagctccggctacggtcgctgccgttaagcctaactctggatgggttgattttactaatcttgctgatttcactttgaacggaaacggagccattttcgacggtcaaggctcactcgcctggaaggccaatgactgcgccaaaactggaaagtgcaactctctcccccattttcctgtgaatgttttcagacatgggtttggacataaatcacaacaagataagacaagaagaatggctgatgacaaatggaccgatgaagaagttaggtatttctttgctctttatgctgatgagaaaaagaaagggaacagaccaaggagtggcatgaatctagctggaagagagttcatcgtaaataagtttgaagagaaatttggtaagagatggatatgggacaggtttaagaacaaggttgatattagtagaaaagcatatgtcaagttcaagaagcttacccacaatagaaccgggcttgtctatgatgctttgggaaggttggagatgtcggatgcttggtgggatcaacgcattgcggtaagtatctttctcttagtcttcagtgtattagagttctctttctgcatttaa

SEQ ID NO: 78 - protein, 361 aa, Brassica napus
MVGLRRAKTVLLVLVVVISNTIADGAPLDRGPKGSGAAAPLGATVPGGGNAAAPVGAATTAPAGAKGSPAAAPVGATAPAAAAGGLAAAPAGAAGAAGSLDVKAAGAKGDGTTDDTAAFTAVWKTACEAPGPSTITVPKGDYLVNNIEFLGPCKGPVTFEMSGNMKAPATVAAVKPNSGWVDFTNLADFTLNGNGAIFDGQGSLAWKANDCAKTGKCNSLPHFPVNVFRHGFGHKSQQDKTRRMADDKWTDEEVRYFFALYADEKKKGNRPRSGMNLAGREFIVNKFEEKFGKRWIWDRFKNKVDISRKAYVKFKKLTHNRTGLVYDALGRLEMSDAWWDQRIAVSIFLLVFSVLEFSFCI

SEQ ID NO: 79 - DNA, 465 bp, Brassica napus
atgggacaagactatagctacagccagccttcttcatcttcggagtttgacatgacctccctactcttagcagaagctaaagcctacgcggatgaagctgagagtagctacccaattgaagagccggttcagtacccactgcaacctgaggcagatgaaggaatcccgacgacatgctattgtggtgctgagccagttgttgaaacttcgtacactcgtagagatccaggcagaaggtacttctcgtgcgtcaacgttgacgatggagactgtcacatttggaagtggtgggatgtggcgatcatggaggagatgcgtgactttcagagacaaatcaggctgctcaagaatcaattctttgagactgaccagaaggtggcaaacggacggtatatatcccaaatggtcaacatttatccaatctatcccactccctcaatctccgaaacaagagttatttgctaa

SEQ ID NO: 80 - protein, 154 aa, Brassica napus
MGQDYSYSQPSSSSEFDMTSLLLAEAKAYADEAESSYPIEEPVQYPLQPEADEGIPTTCYCGAEPVVETSYTRRDPGRRYFSCVNVDDGDCHIWKWWDVAIMEEMRDFQRQIRLLKNQFFETDQKVANGRYISQMVNIYPIYPTPSISETRVIC

SEQ ID NO: 81 - DNA, 648 bp, Brassica napus
atggtgctaaaggtgtacggacctcactttgcttcaccaaagcgtgctttggtaacgcttatcgagaagggcgttcccttcgagaccgtccccgtcgatctcatgaaaggagagcacaagcagcctgcttatctcgccttacagcctttcggtaccgttcctgctgttgtcgacggtgactacaagatcttcgagtcacgtgcggtgatgaggtacgtagctgagaagtacaggtcacaaggacctgaccttttgggcaaaaccgtggaagacagaggtcaagttgagcaatggctcgacgttgaggccaccactttccacccacctctacttaacctcacactccacatcatgttcgcatcagtcatgggattcccttccgatgagaagctgatcaaggagagtgaggagaagctctcagctgtgctcgatgtctacgaggcacatctctccaagagcaagtacttggctggtgatttcgtcagcttggctgatttggctcacctccctttcactgattacttggttggacccatcggtaaggcttacatgatcaaagacaggaagcacgtgagcgcatggtgggatgatattagcagccgtcctgcttggaaggagacacttgagaagtactcattccctgcttaa

SEQ ID NO: 82 - protein, 215 aa, Brassica napus
MVLKVYGPHFASPKRALVTLIEKGVPFETVPVDLMKGEHKQPAYLALQPFGTVPAVVDGDYKIFESRAVMRYVAEKYRSQGPDLLGKTVEDRGQVEQWLDVEATTFHPPLLNLTLHIMFASVMGFPSDEKLIKESEEKLSAVLDVYEAHLSKSKYLAGDFVSLADLAHLPFTDYLVGPIGKAYMIKDRKHVSAWWDDISSRPAWKETLEKYSFPA

SEQ ID NO: 83 - DNA, 1584 bp, Brassica napus
atgaaggatacaatctctgttttgtgtcttgttcttttggtttcggttttagaagtagcaatagcaaaaccgaactctgcaaactttatcgaatgccttcactatcggactagtccgaagaatccagtcaccgatgccatcttcacccccgataacaccaccaccttcttgtcttcttacttgtcctacacgaaaaacaagaggtactcgagccctaaccaaaacctactagccatcgtggtggcagaacatgtatctcatgttcaagccaccgtggtctgcgcaaagaccaacggtatacaactccgtacccgaagtggcggtcatgacctcgaagccctttcttacatatcctctgttccatttgtcattcttgatatgcacaatcttaggtcaattaccattgacgtgccacgcaagaaagcttgggttcaagctggtgccaccttgggagagctctacgtaaagatcaaagacgcaagcaaaacgctagcgttcccagctggcgtgtgccctacggtaggagcaggagggcacataagcggtggagggtacggtaatctcataagaaaatatggaatcactgtggatcatgtcgttgatgctctattagttgatgtcaacggcaagctcttgaaccgagctaccatgggagaagatctcttttgggctatccgtggaggtgggggtgcaagcttcggagttatcctctcttggaaaattaacctcgtggaagttccaaagatcatgacagtgtttagggttaacaaaacattggaacaaggaggcactgatgttctctacaagtggcagcttgtctccaccaagttccctgaagatcttttcattagagcatggcctcaggtcgtaaacggaacaaaacatggcgagagaaccatcgcggttgtgttctacgctcagttcttgggtcgagccgacaagctgatggcgttactaaacaagaacttgcctgggttagggctaaaacgtgaagattgtcacgaaatgagctggttcgaaacgacgctgttttgggctgattaccctgaaggaacaccaccgagcgttcttctagaaagacctacgaatccaggattcttcaaaagcaaatctgattacgtcaagaaacctatccctaaagaagggttcgagaagctctggaaaaaaatgtttgaattcaaacacactgtgtggatgcaaatgaacacttacggtggagtgatggacaggattccggccaacgccacggcgtatcctcatcggaaaggaaacatgttcaaggttcaatactctgcgacatggttggacgcaagcgaaacagagactacccttagactgatgagggagctatatgaggttgcggaaccgtatgtctcaagtaacccgagagaggcgttttttaattacagagatattgatattggaagcaatcctagtggtgagacagacgtggatgaagctaagatctatggtaccaagtatttcttggggaatttgaagagattgatgcaagtaaaagcaaagtatgatcctgagaatttctttaagaacgagcagagtattcctcctgttcggttcatgtag

SEQ ID NO: 84 - protein, 527 aa, Brassica napus
MKDTISVLCLVLLVSVLEVAIAKPNSANFIECLHYRTSPKNPVTDAIFTPDNTTTFLSSYLSYTKNKRYSSPNQNLLAIVVAEHVSHVQATVVCAKTNGIQLRTRSGGHDLEALSYISSVPFVILDMHNLRSITIDVPRKKAWVQAGATLGELYVKIKDASKTLAFPAGVCPTVGAGGHISGGGYGNLIRKYGITVDHVVDALLVDVNGKLLNRATMGEDLFWAIRGGGGASFGVILSWKINLVEVPKIMTVFRVNKTLEQGGTDVLYKWQLVSTKFPEDLFIRAWPQVVNGTKHGERTIAVVFYAQFLGRADKLMALLNKNLPGLGLKREDCHEMSWFETTLFWADYPEGTPPSVLLERPTNPGFFKSKSDYVKKPIPKEGFEKLWKKMFEFKHTVWMQMNTYGGVMDRIPANATAYPHRKGNMFKVQYSATWLDASETETTLRLMRELYEVAEPYVSSNPREAFFNYRDIDIGSNPSGETDVDEAKIYGTKYFLGNLKRLMQVKAKYDPENFFKNEQSIPPVRFM

SEQ ID NO: 85 - DNA, 1440 bp, Brassica napus
atggagagagcaaagtcgagaaagcctcatgccatgatgataccattcccacttcaaggccatgtcatcccttttgtccacttagccatcaaactagcttcacatggcttcaccattactttcgtcaacaccgattccatccaccaccacatctccaccgctcgccaaggtgacgccggagatatcttctccgccgctcgaacctccggaaaccttgacatacgttacaccacggtgagcgacggcttccctttggagtttgaccggtcgcttaaccatgaccaattcttcgaaggccttttccacgtctttcctgcccacgttgatgatctcatcaccaaaatctcccgccggggcgatgatcctccggtgacttgctttatcgccgacacgttttacgtttggtcatctatgatttgtaacaagcacaacctcgttaatgtctcgttttggacccaacctgccttggcccttaatatctattatcacttgcatctcctcatatccaacggccatttcaattctcttgataatcgtgaagacgtgatcgattacataccaggagttaaggcaatagatccaaaggacttgatgtcatatcttcaagtgagcgacaaagacgtcgacacaaacagagtggtctatagaataatattcgaggccttcacagacgtcaagaaagcagatttcgttttatgcaacaccgtacaagagctagaaccaggctctctctctgctctacaagccaaccaacctgtttacgccatcggtcccattttctcaaccgaatcggttgtgccaacaagcctgtgggccgagtctgattgtaccgagtggctcaaaggccggcccaccggatcagtcctctacgtctcgtttggtagctatgcccatgttggtaagaaggagatagtggagatagcacatgggcttttgctaagtggagttagtttcatttgggttttacgtccagatatagttagctctgacgtgcaggattttcttccaactgggtttatggaccgagcccaaagtcgaggtatcgtggtccagtggtgttgtcaaatggcagtaatctcaaacccggccattggagggtttttgacacaatgcgggtggaattcggttctagaaagcgtttggtgtggtttaccattgttgtgttatccgcttttaaacgatcagttcacgaaccggaagcttgtggtggatgattggcgcactggaattaacctttgtgagaacaagatggtcacaagggatgaagtctcggtgaatattaagcggttgatgaacgaagaaactttaagtgagctgagaagcaacgctgagaaggttaatcgtcatatcaaagatgcggttacaaccgttggatcttcagaggttaattttaactcattcgttggcgatgtacaagatagaatagaaattagaaactga

SEQ ID NO: 86 - protein, 479 aa, Brassica napus
MERAKSRKPHAMMIPFPLQGHVIPFVHLAIKLASHGFTITFVNTDSIHHHISTARQGDAGDIFSAARTSGNLDIRYTTVSDGFPLEFDRSLNHDQFFEGLFHVFPAHVDDLITKISRRGDDPPVTCFIADTFYVWSSMICNKHNLVNVSFWTQPALALNIYYHLHLLISNGHFNSLDNREDVIDYIPGVKAIDPKDLMSYLQVSDKDVDTNRVVYRIIFEAFTDVKKADFVLCNTVQELEPGSLSALQANQPVYAIGPIFSTESVVPTSLWAESDCTEWLKGRPTGSVLYVSFGSYAHVGKKEIVEIAHGLLLSGVSFIWVLRPDIVSSDVQDFLPTGFMDRAQSRGIVVQWCCQMAVISNPAIGGFLTQCGWNSVLESVWCGLPLLCYPLLNDQFTNRKLVVDDWRTGINLCENKMVTRDEVSVNIKRLMNEETLSELRSNAEKVNRHIKDAVTTVGSSEVNFNSFVGDVQDRIEIRN

SEQ ID NO: 87 - DNA, 1215 bp, Brassica napus
atggaagtacttggcaccctagatattgtcatcgttggcgccggaatctccggcctttcaaccgccgttggactccataggcttgggattagaagcatggtgctggaatcttcagataagctgagagccacaggattcgcatttactacttggttcaacgcttggaaggctatggaagctctcggcgtttctcagcatgttcgtgatctccatgatctccttcaaggatgggtggtgggacacatctccccaggaaatccttccaaggaaatgctctttccaaaatccgaagaatacgagtctcgatgcgtacagaggaaggtcctgttagaggctctagcggatgagttgcctcaagggaccataaggttttcgtctaaggttgttcatatcgaattgtctggatactacaagatggttcatctctctgatggcactattctcaaaaccaaggttttggtagggtgtgatggtgtgaagtcagtggttggtaagtggctaggcttcaagaatccggctacaacttcccggttagcaatccgagggctcacacattttccacaaggccatggatttgggaaaaagttcttccagttttatggcaacggtgttcgctcgggttttatcccctgtgaccacaacactgtctactggttcctaacccacactggtattgaattagatgaggagacaagtcccgaaaacatcaaagaatttgtgctgaacaagatcaaagacttgcctgaaaacattaagagtgtggtggagaccaccgatcttgatagcatggtgatgtctcgactcaagtaccgacccccatgggaactcttatgggcaaacatcgcaaaggacaacgtatgcgttgcaggggatgcacttcacccaatgactccagatataggacagggcggttgctcagccatggaggatggagttatcctggctcgttgtctcggtgaagctataaaagctaaaggtgaaacagaggatgaaggggagagatataagcggattgaacaaggtctgaagaagtacgcaggagaaaggaaatggagaagcatagatctgataacaacgtcatatacagtaggattcatacagcagagcacagggaagtggatgaacctgttgagagacaagttcttgtcctctttcctttcttggttgctgctgaaaaagtctcatttcgactgcggaagcctcgtccccacatga

SEQ ID NO: 88 - protein, 404 aa, Brassica napus
MEVLGTLDIVIVGAGISGLSTAVGLHRLGIRSMVLESSDKLRATGFAFTTWFNAWKAMEALGVSQHVRDLHDLLQGWVVGHISPGNPSKEMLFPKSEEYESRCVQRKVLLEALADELPQGTIRFSSKVVHIELSGYYKMVHLSDGTILKTKVLVGCDGVKSVVGKWLGFKNPATTSRLAIRGLTHFPQGHGFGKKFFQFYGNGVRSGFIPCDHNTVYWFLTHTGIELDEETSPENIKEFVLNKIKDLPENIKSVVETTDLDSMVMSRLKYRPPWELLWANIAKDNVCVAGDALHPMTPDIGQGGCSAMEDGVILARCLGEAIKAKGETEDEGERYKRIEQGLKKYAGERKWRSIDLITTSYTVGFIQQSTGKWMNLLRDKFLSSFLSWLLLKKSHFDCGSLVPT

SEQ ID NO: 89 - DNA, 1080 bp, Brassica napus
atgggatcaccagtggcatttttggcagcagctttgcttgtcttagcattatctcttggttttgtatctgaaaccaccgcaaattactattattcttctcctcctccgccggtgaagcactacactcctccagtttacaaatctccaccaccacttattaaacactatccagctcctccagtttacaaatctcccccaccacccaagaagcaatacgaatacaaatcacctccaccaccggtaaagcattactcccctcgaccggtttacaagtctcctccaccaccgaagaagcactacgagtacaaatcaccaccaccaccggtctaccagtctcctcctcctccagtttaccactctccaccaccaccaaagaagcactacgagtacaaatcaccaccaccaccggtctaccagtctcctcctcctccagtgtaccactctccaccaccaccaaagaagcactacgagtacaaatcaccaccaccaccggtctaccagtctcctcctcctccagtgtaccactctccaccaccaccaaagaagcactacgagtacaaatcaccaccaccaccggtctaccagtctcctcctcctccagtgtaccactctccaccaccaccaaagaagcactacgagtacaaatcaccaccaccaccggtctaccagtctcctcctcctccagtttaccactctccaccaccaccaaagaagcactacgagtacaaatcaccaccaccaccggtctaccagtctcctcctcctccagtgtaccactctccaccaccaccaaagaagcactacgagtacaaatcaccaccaccaccagtctaccagtctcctcctcctccagtttaccactctcctccaccaccaaagaagcattatgagtacaaatctcctcctcctccagttttccagtctcctcctcctccggtttaccattctcccccaccaccaaaacactatgaatacaaatctcctcctcctcccgtccactctcctcctccaccggttcactactcacctcctcaccaaccctacctctacaaatctccacctcctccataccactactaa

SEQ ID NO: 90 - protein, 359 aa, Brassica napus
MGSPVAFLAAALLVLALSLGFVSETTANYYYSSPPPPVKHYTPPVYKSPPPLIKHYPAPPVYKSPPPPKKQYEYKSPPPPVKHYSPRPVYKSPPPPKKHYEYKSPPPPVYQSPPPPVYHSPPPPKKHYEYKSPPPPVYQSPPPPVYHSPPPPKKHYEYKSPPPPVYQSPPPPVYHSPPPPKKHYEYKSPPPPVYQSPPPPVYHSPPPPKKHYEYKSPPPPVYQSPPPPVYHSPPPPKKHYEYKSPPPPVYQSPPPPVYHSPPPPKKHYEYKSPPPPVYQSPPPPVYHSPPPPKKHYEYKSPPPPVFQSPPPPVYHSPPPPKHYEYKSPPPPVHSPPPPVHYSPPHQPYLYKSPPPPYHY

SEQ ID NO: 91 - DNA, 801 bp, Brassica napus
atggagtctgctaaatcaaatatgaatggccaccaaaacatcatcgtgatgcgccacggtgatcggctcgaccattgtaagccactatgggtttcaaccgctgagagaccgtgggatcctccgctcgtacacgatggtaaggttcgagcctatcaaaccggtcaaagaatccgatctcagattgggtttccgattcaccgtgtctttgtttctcctttcctccgctgcatccagaccgctgccgaagtcgtcgccgctctctcagccgacgatctcgatgacaacgctatgccttccatcgatatctctaagctcaaggtggctattgaatttggattgtgcgagacattgaacacaatggctattaagagtgacgttgttcccaaagatgggaagtttgatttcaagttttcagatcttgaagctatgtttcctgagggaacatttgatcacaatgtggaaatggcttataaagagtttccacagtggggagaatctgtggaagactttaaagaaagatatgttaatacattgaagattcttgcagagaagtatccttcagagaatttgttactagtcacccactggggaggtgtaagttctatgctttacaaatacttcaaagacgcaactaagtacttagtagactactgtggttgtgttgagttgagaaggcagattatggataatgatggatttggtgaatctgcggactttgaggtggttacaagtcatggtgtagctttcaagaacaacaaagccccgattcatggtcctcttataatccaatctcccatttag

SEQ ID NO: 92 - protein, 266 aa, Brassica napus
MESAKSNMNGHQNIIVMRHGDRLDHCKPLWVSTAERPWDPPLVHDGKVRAYQTGQRIRSQIGFPIHRVFVSPFLRCIQTAAEVVAALSADDLDDNAMPSIDISKLKVAIEFGLCETLNTMAIKSDVVPKDGKFDFKFSDLEAMFPEGTFDHNVEMAYKEFPQWGESVEDFKERYVNTLKILAEKYPSENLLLVTHWGGVSSMLYKYFKDATKYLVDYCGCVELRRQIMDNDGFGESADFEVVTSHGVAFKNNKAPIHGPLIIQSPI

SEQ ID NO: 93 - DNA, 201 bp, Brassica napus
atgtcgttgagcaattacactgccgtaatcgccttgatcgttttcgccgtcgtgtctgccggcgctcaatccctagctcctgctccatctcccacaagcgacggaacatcgatcgatcaaggaatagcgtatttgctaatggtggtggcgttggtgctgacgtatatcattcatcccctcgatgcatcttctttcttctga

SEQ ID NO: 94 - protein, 66 aa, Brassica napus
MSLSNYTAVIALIVFAVVSAGAQSLAPAPSPTSDGTSIDQGIAYLLMVVALVLTYIIHPLDASSFF

SEQ ID NO: 95 - DNA, 894 bp, Brassica napus
atggagtttactacaaaaataacatcattctctctcttatttctctctctcataaacccgaccttgaccactgccacatgcagtaccgccgtttgtcgcaatggcgatccaattatccgtttccctttccgtttaaaatcccaccaaccccaatcttgcggttatgacaaagggttcgacctaacctgcggtaataataacggcgttaacagaaccaccataagactaccattctctggtaacttcaccatcgagatgatcgattacgcagctcaagagattttactcgccgacccaaataactgtcttcctaaacgcctcttaacgctaaacctcacttcgacgccgtttgatggcgtttacacacgtcggttcacgttttttagcttcccgacgtcgggatatcttcgttttgggaggttcaatccgataacgtgtttgagcgatgaaaacagcaccgtgtttgccactgcttttcctagagcggtgaactacctgtccgctcagtcgtgccggttaatgaaaaccgttaacgttccggttcgttggccggcttatgagcatgccgtttcgtattggggactcagtgataacctatggctcacttggagggtcccgagatgccgccggtgtgagagtagaggtggtaagtgtgggattaagagtaattcttctcgtgaaatcatatgctctgatgctcctaagccaggcgcaagaagagaccgtagtttgagacacgtgttggacaaaagggctacatctctttccctcttcccactccgtgtctctctctctccctttcgtcgaccacaccgcttctctttctctcttctcctcgaggactccgcttccctttgttcatctctcaacgatcggatgaatcagccgagatga

SEQ ID NO: 96 - protein, 297 aa, Brassica napus
MEFTTKITSFSLLFLSLINPTLTTATCSTAVCRNGDPIIRFPFRLKSHQPQSCGYDKGFDLTCGNNNGVNRTTIRLPFSGNFTIEMIDYAAQEILLADPNNCLPKRLLTLNLTSTPFDGVYTRRFTFFSFPTSGYLRFGRFNPITCLSDENSTVFATAFPRAVNYLSAQSCRLMKTVNVPVRWPAYEHAVSYWGLSDNLWLTWRVPRCRRCESRGGKCGIKSNSSREIICSDAPKPGARRDRSLRHVLDKRATSLSLFPLRVSLSPFRRPHRFSFSLLLEDSASLCSSLNDRMNQPR

SEQ ID NO: 97 - DNA, 744 bp, Brassica napus
atggcgcagccttatgtgtacgctcacccaccaggaacggctcctaggccttcgggagcgttcgtgaatccaaggttctgtgttgcgggccccgtggatctgacgatggttcgggatgagactgagaaaaaatggggtagtttctatatactggacgctaacatgaacctgcggtttcaggtgaagaagccgggttttggttttggttttggcaggaatatgattttattggatggttctggatctccgatcttgactatgaaagagcagacgatgaccatgagcttccgtgagaagtgggaagtacatataggggatcaagcggcatacacggtgaagggatcgtcgattttttcgagtagtacgaaacttgatggtcttcatgtgtttttggctcgaaaccatgaagaggagataccagatttcagagttaaagcgacaaatcaccgtgggtttgaacgctcttgcgtcgtctacgccggtgaatctgacaccattgttgcccaaatgcagcacgaggacgtcacggcgagtaacaccgacattttcacggtgacgattaatccaaacgtcgatcatgccttcattgcctctctcgtaatcattctcgatgtttataaccgagtagatacagagttgcctcataggtaccatgaggcacaccaggctgtaaaccgtgttcatatgggtttacacggtgctacacacgctgctctacgcgctggcgcttgcactatacaataa

SEQ ID NO: 98 - protein, 247 aa, Brassica napus
MAQPYVYAHPPGTAPRPSGAFVNPRFCVAGPVDLTMVRDETEKKWGSFYILDANMNLRFQVKKPGFGFGFGRNMILLDGSGSPILTMKEQTMTMSFREKWEVHIGDQAAYTVKGSSIFSSSTKLDGLHVFLARNHEEEIPDFRVKATNHRGFERSCVVYAGESDTIVAQMQHEDVTASNTDIFTVTINPNVDHAFIASLVIILDVYNRVDTELPHRYHEAHQAVNRVHMGLHGATHAALRAGACTIQ

SEQ ID NO: 99 - DNA, 675 bp, Brassica napus
atgtcaatccgagtcccctcggcttctcaggattctagaggtttcatattatgtatcatcttcttcgggtcgttcccaggtagaacaatacttacacaagacgttaaactagattcgattctgattttcaagacgcatgagtggttctcgaccaagcctatagtttatttccaatgcaaaggagagaacaagactttgtttcccgatgtgaagacaacgaacgtgtcttattctttcagtggccaagaatcttggcagccactaacaaaacttaagggaacaaaatgcaagatatgtggaatctatgaggaagatacctttagatatgatacatttaacgaatgggagctttgtccttctgatttcacacctgagggtacatacacacatgctaaggagaaagatttcaatgctacttttctctgccatggttgctcacaactcggagctggtttgaataaagattctggcactgacaaggaagaagaaacgcgcggaatgcatcatgcaatcgttgtactgattgtagtacttgtgttaggtgtagttgcggtgggtcttgtggtaggtagtacgtactggcaaaagaagaagcggcaacaagagccgacccagtttctgtttgaagatagtgacgaaatggaggacgaacttggcctagacgataccttatga

SEQ ID NO: 100 - protein, 224 aa, Brassica napus
MSIRVPSASQDSRGFILCIIFFGSFPGRTILTQDVKLDSILIFKTHEWFSTKPIVYFQCKGENKTLFPDVKTTNVSYSFSGQESWQPLTKLKGTKCKICGIYEEDTFRYDTFNEWELCPSDFTPEGTYTHAKEKDFNATFLCHGCSQLGAGLNKDSGTDKEEETRGMHHAIVVLIVVLVLGVVAVGLVVGSTYWQKKKRQQEPTQFLFEDSDEMEDELGLDDTL

SEQ ID NO: 101 - DNA, 255 bp, Brassica napus
atggcatcttctaaagtatcttccatgcttctctttctcctccttctcgttcttgtgtttccccatatggataaagcccttggtgaagaaagacagctccacgaacttaaaggcacagagcatcccgatcaactgatgacggttggaaggagccttctctttcgcatccctccatgcccaccgtccttatgtaagcctcgtccgataagaattcctccccgacttcgtacacctccaccccctccacggcattga

SEQ ID NO: 102 - protein, 84 aa, Brassica napus
MASSKVSSMLLFLLLLVLVFPHMDKALGEERQLHELKGTEHPDQLMTVGRSLLFRIPPCPPSLCKPRPIRIPPRLRTPPPPPRH

SEQ ID NO: 103 - DNA, 297 bp, Brassica napus
atgccgggagggcactacaaattggtacatatgataggcatcggtttggttaacaatgtcccgagggcgacttcagcctacgctgctgcaaggcaagaagattctcagctgtgtgtggatttgacggcggccaacaacgtcattgcattgctctgggaagaacaagatgacatgagggctcagttgagggccatggaaaggatgttcgatgtcatcgcatcagggaacccagagattatgtgggcatgggattctgttcgtcagtttgttcaccgcgtccctaccctagaggagtag

SEQ ID NO: 104 - protein, 98 aa, Brassica napus
MPGGHYKLVHMIGIGLVNNVPRATSAYAAARQEDSQLCVDLTAANNVIALLWEEQDDMRAQLRAMERMFDVIASGNPEIMWAWDSVRQFVHRVPTLEE

SEQ ID NO: 105 - DNA, 123 bp, Brassica napus
ttgatttggtggtgcattacctggtgcttcactcccatattgcccttgagcaagtatcccattccaacctgcagttataaacacactcttaacaatattcaatatatatacacatgttccttt

SEQ ID NO: 106 - protein, 41 aa, Brassica napus
MIWWCITWCFTPILPLSKYPIPTCSYKHTLNNIQYIYTCSF

SEQ ID NO: 107 - DNA, 414 bp, Brassica napus
atggtgttactggacgagaagcatgtgactgaatatcatttgcatcttcgcagggatgtgaacttctgtgttagtatatttgactctttgcctcttgcatttcataacaaattcgaaagctacgggtctgagccaaaaattgtggttgccaccagtaaatccgaagatagttggagattaacaggtgatggaacagagcagacagcatcttcatcaaagataattgagtttctctgcacagccaaagtgaccggtgttcagttggtgttatattggctgctccaaatgttcaaagaaactcctgtagcgtgtggaactgtcgttgtcagaccacacaaatattgtctcaggtcaacctcgctttctctgcatacattgcaggtaacggcttgggacagcactccagttttttaa

SEQ ID NO: 108 - protein, 137 aa, Brassica napus
MVLLDEKHVTEYHLHLRRDVNFCVSIFDSLPLAFHNKFESYGSEPKIVVATSKSEDSWRLTGDGTEQTASSSKIIEFLCTAKVTGVQLVLYWLLQMFKETPVACGTVVVRPHKYCLRSTSLSLHTLQVTAWDSTPVF

SEQ ID NO: 109 - DNA, 20 bp, artificial
  Primer
tctcgttgacccaaaggttc

SEQ ID NO: 110 - DNA, 20 bp, artificial
  Primer
cagccttcgctcaaagctac

SEQ ID NO: 111 - DNA, 20 bp, artificial
  Primer
gctgcttttgaagcaccaac

SEQ ID NO: 112 - DNA, 20 bp, artificial
  Primer
gttgcaagatccatgtcgtg

SEQ ID NO: 113 - DNA, 20 bp, artificial
  Primer
tcaacgcgtgtctcattctc

SEQ ID NO: 114 - DNA, 20 bp, artificial
  Primer
taccgggaaaagaggttgtg

SEQ ID NO: 115 - DNA, 20 bp, artificial
  Primer
tcgtctaggccaagttcgtc

SEQ ID NO: 116 - DNA, 20 bp, artificial
  Primer
aaagaagaagcggcaacaag

SEQ ID NO: 117 - DNA, 20 bp, artificial
  Primer
ttggcacttccccacttaac

SEQ ID NO: 118 - DNA, 20 bp, artificial
  Primer
gcgtatcttggaccgatcac

SEQ ID NO: 119 - DNA, 20 bp, artificial
  Primer
gttgggagccttaggaaacc

SEQ ID NO: 120 - DNA, 20 bp, artificial
  Primer
accgtccatcatctgctctc

SEQ ID NO: 121 - DNA, 20 bp, artificial
  Primer
taggctgtgacgggactacc

SEQ ID NO: 122 - DNA, 20 bp, artificial
  Primer
tccggcttcatagaatgtcc

SEQ ID NO: 123 - DNA, 20 bp, artificial
  Primer
ttcaccgacctccttgcttc

SEQ ID NO: 124 - DNA, 23 bp, artificial
  Primer
gaagctgctgcgagaagattgcg

SEQ ID NO: 125 - DNA, 20 bp, artificial
  Primer
cttggcattggtgcaactcc

SEQ ID NO: 126 - DNA, 20 bp, artificial
  Primer
tccgagarcgaatccgcttg

SEQ ID NO: 127 - DNA, 20 bp, artificial
  Primer
atctcttggttctggcatcg

SEQ ID NO: 128 - DNA, 20 bp, artificial
  Primer
gcaatgtgcgttcaaagatt

(19) United States
(12) Patent Application Publication
(10) Pub. No.: US 2021/0108222 A1
(43) Pub. Date: Apr. 15, 2021

(54) PLANTS HAVING INCREASED RESISTANCE TO L. MACULANS AND METHODS OF USE

(71) Applicant: University of Manitoba, Winnipeg (CA)

(72) Inventors: Michael Becker, Winnipeg (CA); Mark Belmonte, Winnipeg (CA); Dilantha Fernando, Winnipeg (CA); Xuehua Zhang, Winnipeg (CA)

(21) Appl. No.: 16/332,593

(22) PCT Filed: Sep. 13, 2017
(86) PCT No.: PCT/IB2017/055511

§ 371 Date: Mar. 12, 2019

Related U.S. Application Data
(60) Provisional application No. 62/393,966, filed on Sep. 13, 2016.

(51) Int. Cl. C12N 15/82; A23L 33/105; A23K 10/30; A23D 9/02; C07K 14/415

(52) U.S. Cl. CPC C12N 15/8282; A23L 33/105; C07K 14/415; A23D 9/02; A23K 10/30

(57) ABSTRACT

Provided herein is a transgenic plant having increased expression of a coding region encoding a resistance protein, where the transgenic plant has increased resistance to infection by Leptosphaeria maculans. In one embodiment, the transgenic plant is B. napus, B. oleraceae, B. rapa, or B. juncea. Also provided are methods of increasing resistance of a member of the genus Brassica to infection by Leptosphaeria maculans, methods of making a transgenic plant with increased resistance to Leptosphaeria maculans, and methods of producing food, feed, or an industrial product using a transgenic plant.

### CROSS-REFERENCE TO RELATED APPLICATIONS

This application claims the benefit of U.S. Provisional Application Ser. No. 62/393,966, filed Sep. 13, 2016, which is incorporated by reference herein.

### SUMMARY

Provided herein is a transgenic plant having increased expression of a coding region encoding a resistance protein. The resistance protein is identical to or has structural similarity to a protein selected from SEQ ID NO:2, 4, 6, 8, 0, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, or 108. The amount of the resistance protein in the transgenic plant is increased compared to the wild type plant, the transgenic plant is a member of the genus Brassica, and the transgenic plant includes increased resistance to infection by Leptosphaeria maculans compared to the wild type plant. In one embodiment, the transgenic plant is B. napus, B. oleraceae, B. rapa, or B. juncea.

In one embodiment, the coding region encodes a receptor, such as a receptor selected from SEQ ID NO:22, SEQ ID NO:2, SEQ ID NO:12, SEQ ID NO:4, SEQ ID NO:6, SEQ ID NO:20, SEQ ID NO:26, or SEQ ID NO:18. In one embodiment, the coding region encodes a protein involved in signal transduction and gene regulation, such as the protein SEQ ID NO:38. In one embodiment, the coding region encodes a protein that is a transcription factor, such as a protein selected from SEQ ID NO:32, SEQ ID NO:34, or SEQ ID NO:36. In one embodiment, the coding region encodes a protein associated with sulfur assimilation, such as a protein selected from SEQ ID NO:40 or SEQ ID NO:42. In one embodiment, the coding region encodes a protein catalyzing a step in glucosinolate biosynthesis or indole glucosinolate biosynthesis, such as a protein selected from SEQ ID NO:44, SEQ ID NO:46, SEQ ID NO:48, SEQ ID NO:50, SEQ ID NO:52, SEQ ID NO:54, SEQ ID NO:56, SEQ ID NO:58, or SEQ ID NO:60. Optionally, the transgenic plant includes increased expression of at least two coding regions encoding resistance proteins.

Also provided is a part of a transgenic plant described herein, where the part is a leaf, a stem, a flower, an ovary, fruit, a seed or a callus. A part of a transgenic plant includes an increased amount of a protein encoded by a coding region described herein. Further provided are progeny of a transgenic plant, including but not limited to a progeny that is a hybrid plant.

Also provided herein are methods. In one embodiment, a method is for increasing resistance of a member of the genus Brassica to infection by Leptosphaeria maculans. The method includes increasing in the member of the genus Brassica expression of a coding region encoding a resistance protein identical to or having structural similarity to a protein selected from SEQ ID NO:2, 4, 6, 8, 0, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, or 108.

In one embodiment, a method is for making a transgenic plant with increased resistance to Leptosphaeria maculans. The method includes increasing expression of a coding region encoding a resistance protein identical to or having structural similarity to a protein selected from SEQ ID NO:2, 4, 6, 8, 0, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, or 108, where expression of the protein in the transgenic plant is increased compared to the wild type plant, and where the transgenic plant is a member of the genus Brassica.

In one embodiment, a method is for producing oil. The method includes harvesting seeds from a transgenic plant described herein and extracting the oil from the seeds.

In one embodiment, a method is for producing food, feed, or an industrial product. The method includes obtaining a transgenic plant described herein or a part thereof, and preparing the food, feed or industrial product from the plant or part thereof. In one embodiment, the food or feed is, for instance, oil, meal, grain, starch, flour or protein. In one embodiment, the industrial product is, for instance, biofuel, fiber, industrial chemicals, a pharmaceutical or a nutraceutical.

In one embodiment, a method id for producing an oil. The method includes crushing seeds produced from at least one transgenic plant described herein, and extracting the oil from said crushed seeds.

As used herein, the term “transgenic plant” refers to a plant that has been engineered to have increased expression of one or more coding regions described herein. In one embodiment, cells of a transgenic plant contain a polynucleotide encoding a protein described herein. The term “transgenic plant” includes whole plants, plant parts (stems, roots, leaves, fruit, etc.) or organs, plant cells, seeds, and progeny of same. A transformed plant can be a direct transfectant, meaning that the DNA construct was introduced directly into the plant, such as through Agrobacterium or other methods, or the plant can be the progeny of a transfected plant. The second or subsequent generation plant can be produced by sexual reproduction, i.e., fertilization. Furthermore, the plant can be a gametophyte (haploid stage) or a sporophyte (diploid stage).

As used herein, a “control” plant or “control” host cell refers to a cell that has not been engineered to have increased expression of a coding region described herein. In one embodiment, an example of a control plant or control host cell is one that is wild-type. In one embodiment, an example of a control plant or control host cell is one that is not wild-type (e.g., it is transgenic for some other type of coding region) but has not been engineered to have increased expression of a coding region described herein.

As used herein, the term “infection” refers to the presence of and/or reproduction of L. maculans on or in the body of a plant. The presence of L. maculans on or in the body of a plant is also referred to as colonization. The infection can be clinically inapparent, or result in symptoms associated with disease caused by the microbe. The infection can be at an early stage, or at a late stage. Symptoms include, but are not limited to, necrotic lesions on leaves, often with development within of tiny black, spherical structures of 0.5mm diameter referred to as pycnidia. Stem symptoms include plant lodging, and blackening at the base of the plant and within the stem.

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, tetramers). Thus, the terms peptide, oligopeptide, and protein are all included within the definition of protein and these terms are used interchangeably.

As used herein, a protein may be “structurally similar” to a reference protein if the amino acid sequence of the protein possesses a specified amount of sequence similarity and/or sequence identity compared to the reference protein. Thus, a protein may have structural similarity to a reference protein if, compared to the reference protein, it possesses a sufficient level of amino acid sequence identity, amino acid sequence similarity, or a combination thereof.

As used herein, the term “polynucleotide” refers to a polymeric form of nucleotides of any length, either ribonucleotides, deoxynucleotides, or a combination thereof, and includes both single-stranded molecules and double-stranded duplexes. A polynucleotide can be obtained directly from a natural source, or can be prepared with the aid of recombinant, enzymatic, or chemical techniques.

As used herein, a polynucleotide may have “sequence similarity” to a reference polynucleotide if the nucleotide sequence of the polynucleotide possesses a specified amount of sequence identity compared to a reference polynucleotide. Thus, a polynucleotide be structural similarity to a reference polynucleotide if, compared to the reference polynucleotide, it possesses a sufficient level of nucleotide sequence identity.

An “isolated” polynucleotide or protein is one that has been removed from its natural environment. Polynucleotides and proteins that are produced by recombinant, enzymatic, or chemical techniques are considered to be isolated and purified by definition, since they were never present in a natural environment.

As used herein, the terms “coding region” and “coding sequence” are used interchangeably and refer to a nucleotide sequence that encodes a protein and, when placed under the control of appropriate regulatory sequences expresses the encoded protein. The boundaries of a coding region are generally determined by a translation start codon at its 5′ end and a translation stop codon at its 3′ end.

As used herein, a “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

As used herein, the term “heterologous” refers to a nucleotide sequence that is not normally or naturally found flanking another nucleotide sequence. For instance, a coding region and a promoter may be heterologous.

As used herein, the term “exogenous” refers to a polynucleotide or protein that is not normally or naturally found in a specific plant.

While the polynucleotide sequences described herein are listed as DNA sequences, it is understood that the complements, reverse sequences, and reverse complements of the DNA sequences can be easily determined by the skilled person. It is also understood that the sequences disclosed herein as DNA sequences can be converted from a DNA sequence to an RNA sequence by replacing each thymidine nucleotide with a uridine nucleotide.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1-1 through 1-35 shows proteins and examples of coding regions encoding the proteins.

FIG. 2 shows principle component analysis of raw counts for each individual treatment. Legend on right of graph shows representative color for each treatment group.

FIG. 3 shows RNA quality following tissue processing and laser microdissection. Representative electropherograms were recorded using a microfluidic PicoChip on the Agilent 2100 bioanalyzer. Ribosomal peaks (18S and 25S) and chip marker have been superimposed onto electropherograms to aid interpretation.

FIG. 4 shows disease symptoms in B. napus cotyledons in response to L. maculans infection. (FIG. 4a) Disease symptoms in resistant (R) and susceptible (S) cotyledons at 3, 7 and 11 days post inoculation (dpi). (FIG. 4b) Lesion size over time. Asterisks (p<0.01, student's t test). Scanning electron micrograph (SEM) of R at 3 dpi (FIGS. 4c) and 11 dpi (FIG. 4d) at the infection site (black arrow), scale=1 mm (FIG. 4e) Fungal hyphae (H) at infection site, scale=50 μM in R at 11 dpi. (F) SEM of S at 3 dpi (f) and 11 dpi (FIG. 4g) at the infection site (IS), scale=1 mm (FIG. 4h) SEM of pycnidia (Py) on S cotyledons at 11 dpi, scale=200 μM. (FIG. 4i-n) Light micrographs of R at 3 dpi (FIG. 4i), 7 dpi (FIG. 4j), 11 dpi (FIG. 4k) and S at 3 dpi (FIG. 4l), 7 dpi (FIGS. 4m) and 11 dpi (FIG. 4n). Scale bars=500 μM.

FIG. 5 shows hierarchical clustering and global gene activity in the B. napus-L. maculans pathosystem. (FIG. 5a) Hierarchical clustering of all DEGs detected in dataset. (FIG. 5b) Number of transcripts detected in both genotypes across all treatments. Transcripts with an FPKM>1 are considered to be detected. Detected transcripts are subdivided into low (FPKM≥1, <5), moderate (FPKM≥5, <25), or high (FPKM≥25) detection levels.

FIG. 6 shows upregulated DEGs in resistant (R) and susceptible (S) B. napus cotyledons inoculated with L. maculans as compared to mock inoculated controls. (FIG. 6a-c) Venn diagram showing activated genes at 3 dpi (FIG. 6a), 7 dpi (FIGS. 6b), and 11 dpi (FIG. 6c) in response to L. maculans in R (left), S (right), or shared between both genotypes (intersect). (FIG. 6d) Heatmap of enriched GO terms identified from upregulated genes. Terms are considered enriched at P<0.001. Darker blue color represents a greater statistical enrichment. (FIG. 6e-f) Deposition of lignified plant materials at the site of infection in R (FIG. 6e) and S (FIG. 6f) hosts at 7 dpi. Lignified plant materials appear dark orange/red. (FIG. 6g-h) Aniline blue callose staining of R (FIG. 6g) and S (FIG. 6h) B. napus cotyledons inoculated with L. maculans at 7 dpi. Scales=1 mm

FIG. 7 shows expression levels of hormone biosynthesis genes and hormone signaling markers in response to L. maculans. Heatmap of Loge transcript level fold-change vs. mock controls in resistant (R) and susceptible (S) cotyledons at 3, 7, and 11 days post L. maculans inoculation.

FIG. 8 shows deposition of lignified plant materials at the site of infection in resistant and susceptible hosts. Cotyledons are infected with L. maculans and stained with phloroglucinol-HCl. Lignified plant materials appear dark orange/red. Scales=1 mm

FIG. 9 shows differentially expressed (p<0.05) glucosinolate and indole glucosinolate biosynthetic genes in B. napus cotyledons infected with L. maculans. Changes in expression of biosynthetic gene homologs are shown across their respective biosynthetic pathways. Fluctuations in gene expression are recorded as FPKM [Fragments Per Kilobase of transcript per Million mapped reads] deviation from mock controls. A more intense red color reflects gene activation, a more intense blue color represents gene repression.

FIG. 10 shows transcript levels of transcription factors expressed in response to L. maculans. Heatmap of Loge transcript level fold-change vs, mock controls in resistant (R) and susceptible (S) cotyledons at 3, 7, and 11 days post L. maculans inoculation.

FIG. 11 shows identification of DEGs specific to resistant (R) cotyledons inoculated with L. maculans. (FIG. 11a) Venn diagram showing all genes upregulated in R hosts at 3, 7, and 11 days post inoculation (dpi). (FIG. 11b) Identification of DEGs specific to R hosts (FIG. 11c) Expression profiles of 54 DEGs specific to R hosts. Expression levels are measured in FPKM.

FIG. 12 shows disease symptoms in Arabidopsis following L. maculans infection. (FIG. 12a) Wild-type Col-0 (FIG. 12b,c) at4g39940.1, aps kinase 2 (FIG. 12d) at3g14840.1, lysm interacting kinase 1 (FIG. 12e,f) at4g18250.1, putative receptor (FIG. 12g) at3g53490.1, putative receptor (FIG. 12h) at1g73260.1, kunitz trypsin inhibitor 1, (FIG. 12i) at3g11820, penetration 1 (FIG. 12j) Col-0 water inoculated mock control. Scale bar=1 mm (FIG. 12j) Relative abundance of L. maculans 18s rDNA in each mutant. Asterisk (*) denotes significant difference (p<0.05, student's t test) in fungal load compared to Col-0.

FIG. 13 shows B. napus gene expression following inoculation with L. maculans. Relative transcript abundance of BnPDF1.2, BnRBOHF, BnaC04g27200D, BnAPK2, BnaA03g43720D, BnLIK1, BnPR1, BnWRKY25 and BnCYP79B2 in susceptible (S) and resistant (R) cotyledons as measured 0-200, 200-400, and 400-600 μm from the inoculation site. Actin (GenBank accession number: AF111812.1) was used as the internal control and to normalize expression data. Relative transcript abundance is normalized relative to S mock (0-200 μm) treatment. Error bars represent standard deviation of the mean. For each gene, different lowercase letters indicate significant differences among mean values (one-way ANOVA with Ducan's multiple range test (p<0.05)). The results are based on three replicates in three independent experiments.

### DETAILED DESCRIPTION OF ILLUSTRATIVE EMBODIMENTS

Brassica napus (canola, oilseed rape) ranks second largest in production among oilseed crops worldwide and is under constant threat by the devastating fungal pathogen, Leptosphaeria maculans, the causal agent of blackleg (Stotz et al., 2014, Trends Plant Sci 19: 491-500). Resistance to blackleg mediated by race-specific resistance (R) genes often relies on the interaction between R genes and the corresponding pathogen avirulence (Avr) genes (Larkan et al., 2013, New Phytol 197: 595-605). Research and breeding efforts have identified 14 major R-genes for B. napus that are effective against L. maculans isolates with corresponding Avr-genes from the seedling stage to full maturity. However, absence of either R or Avr gene generally results in a compatible host-pathogen interaction and successful pathogen infection. Each interaction is likely governed by large sets of genes activated over time and under the control of complex gene regulatory networks and signal transduction cascades leading to either plant protection or defeat.

Described herein is the identification of genes regulating the plant-type hypersensitive response in Brassica napus against L. maculans. To identify defense genes, high throughput next generation RNA sequencing was used to examine the transcriptome of a universally susceptible (cv. Westar) and commercially available resistant line carrying LepR1 (line DL15 or DF78). Cotyledons were treated with water (mock) or L maculans (D3) and sequenced using the Illumina Hi-seq 2500 platform. Nine samples of the host-incompatible (resistant) advanced breeding line B. napus DF78 (DL15) (LepR1, Rlm3) were inoculated with Australian L. maculans strain D3 (AvrLepR1, AvrLm5), nine samples of universally susceptible B. napus cv. Westar were inoculated with L. maculans strain D3, and 18 controls; nine samples of mock inoculated Westar and 9 samples of mock inoculated DF78 (controls). A portion of the samples were taken at 0, 3, 7, and 11 days post infection.

Sequencing resulted in >475 million high quality (Phred>30) sequence reads. Reads were aligned to the recently published B. napus genome (v4.1, Chalhoub et al., 2014, Science 345: 950-953) and normalized counts were obtained using the cufflinks RNA-seq data analysis pipeline (Trapnell et al., 2012, Nat Protoc 7: 562-78). A total of 57,654 unique transcripts were detected with a normalized expression of FPKM>1.

Differential gene expression was performed using Cuffdiff, comparing both host-incompatible line DF78 and susceptible cv. Westar to their mock controls. It was hypothesized that if a gene was important before, during and after the infection process it would be active across all infection time points (shared over time). A total of 1221 genes with significantly increased transcript levels at every time point in resistant line DF78. To identify those specific to the incompatible interaction any gene from this list that had significantly increased expression at any given time point in susceptible cv. Westar was removed. This produced a dense list of 54 core defense genes that may be contributing to host resistance.

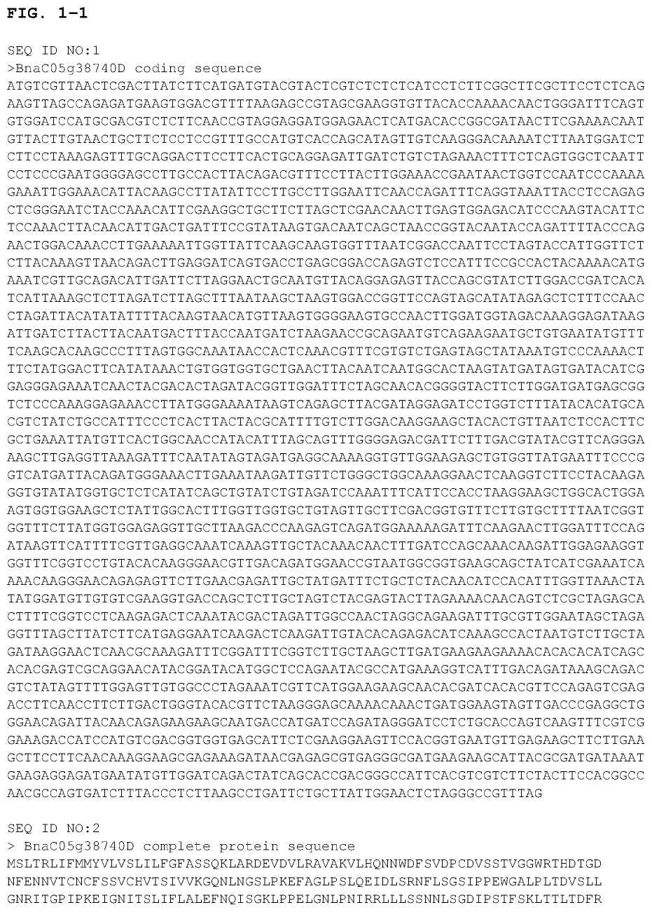

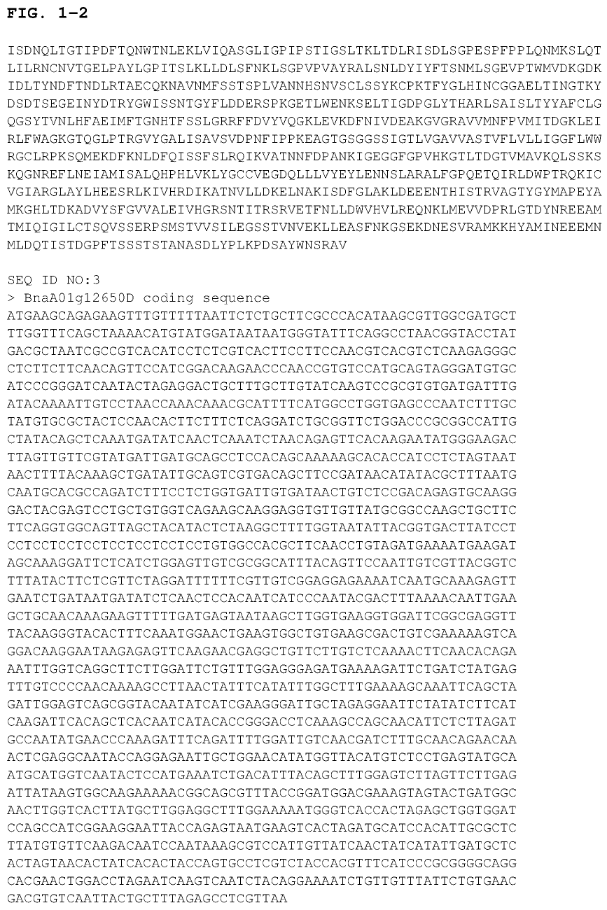

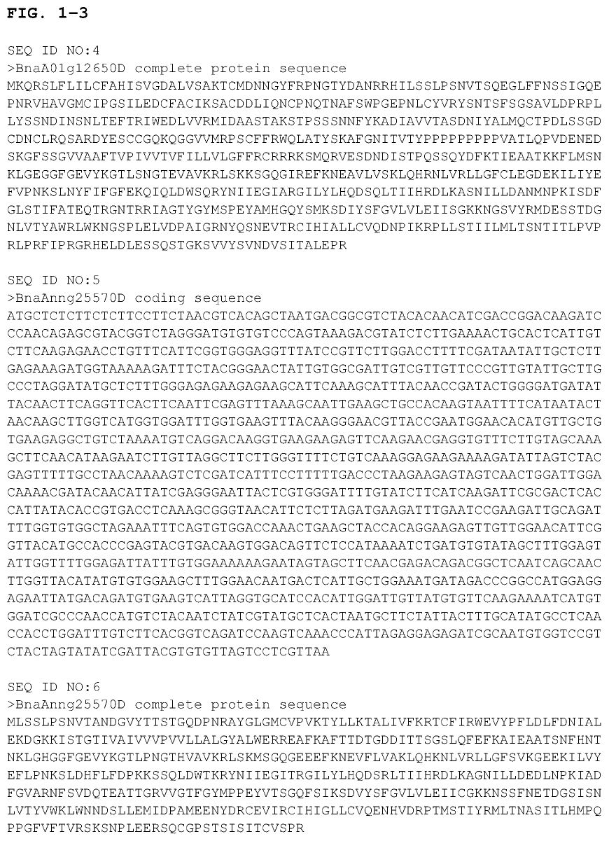

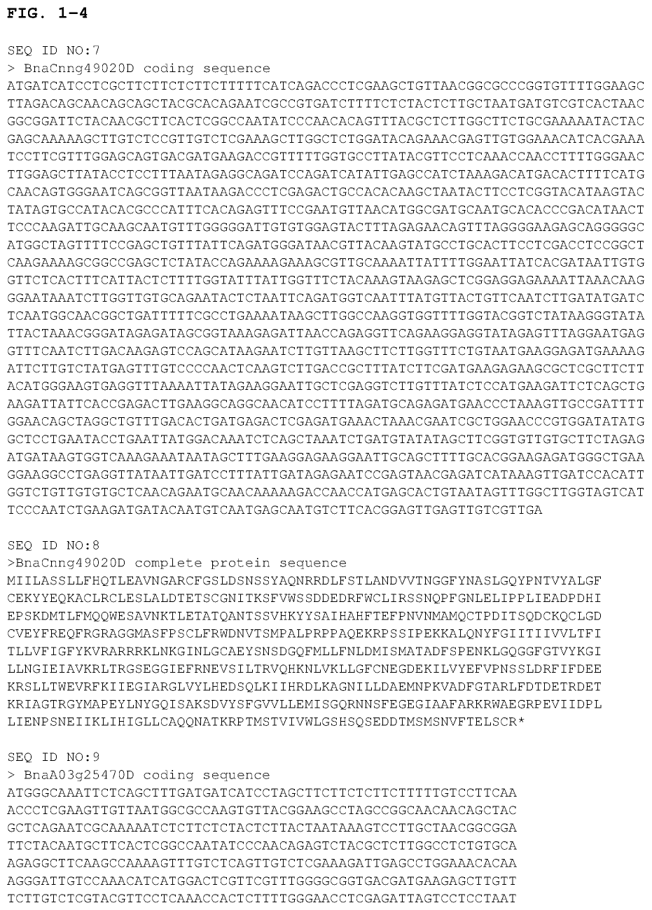

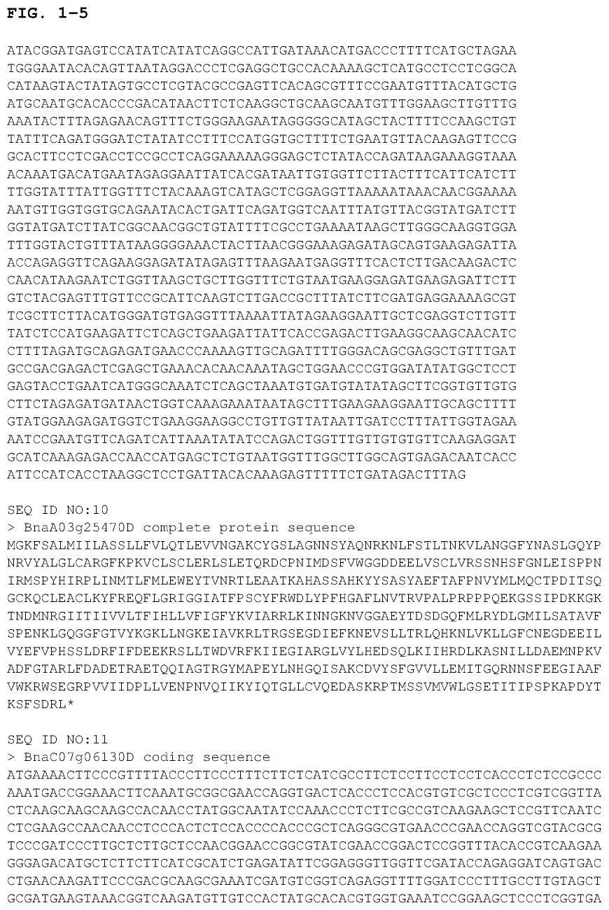

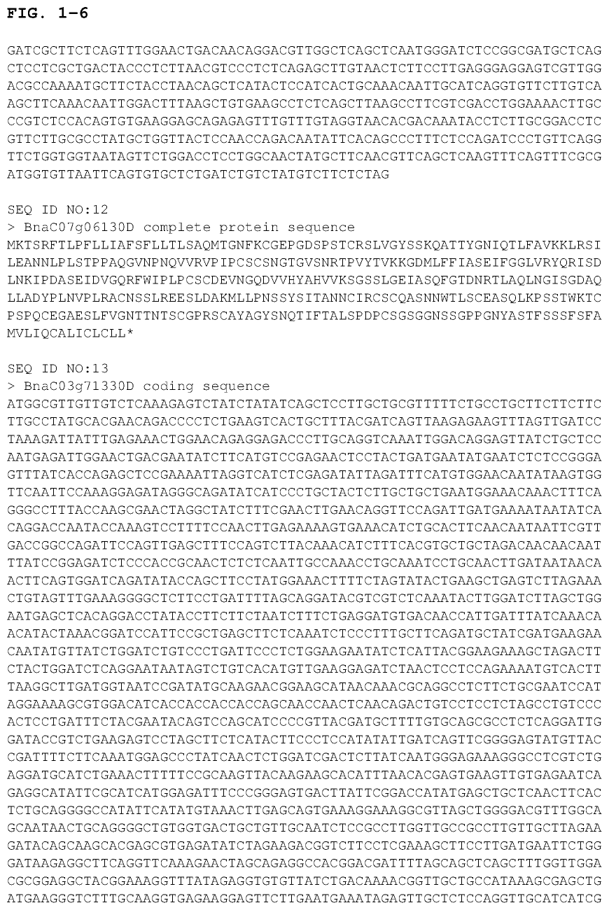

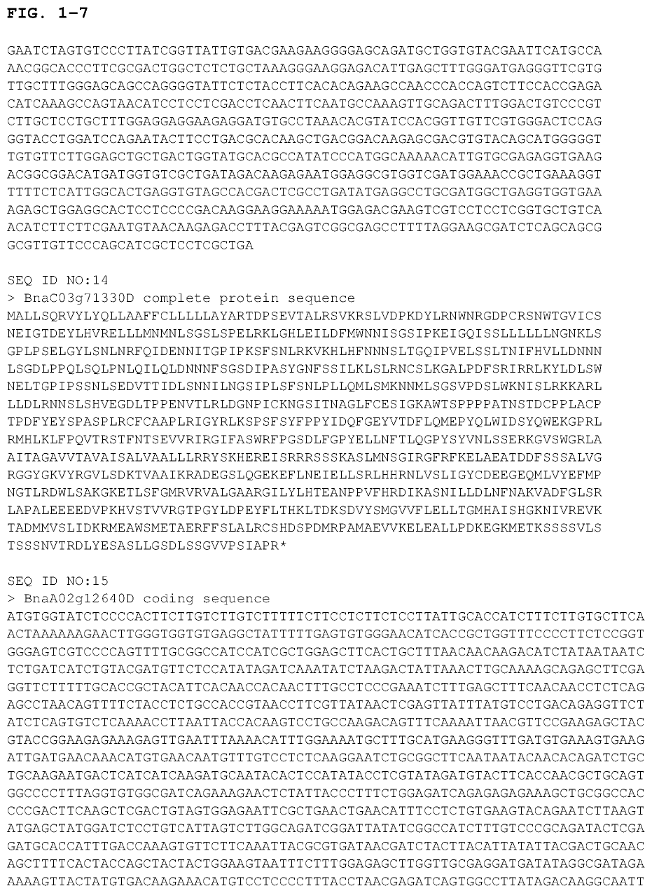

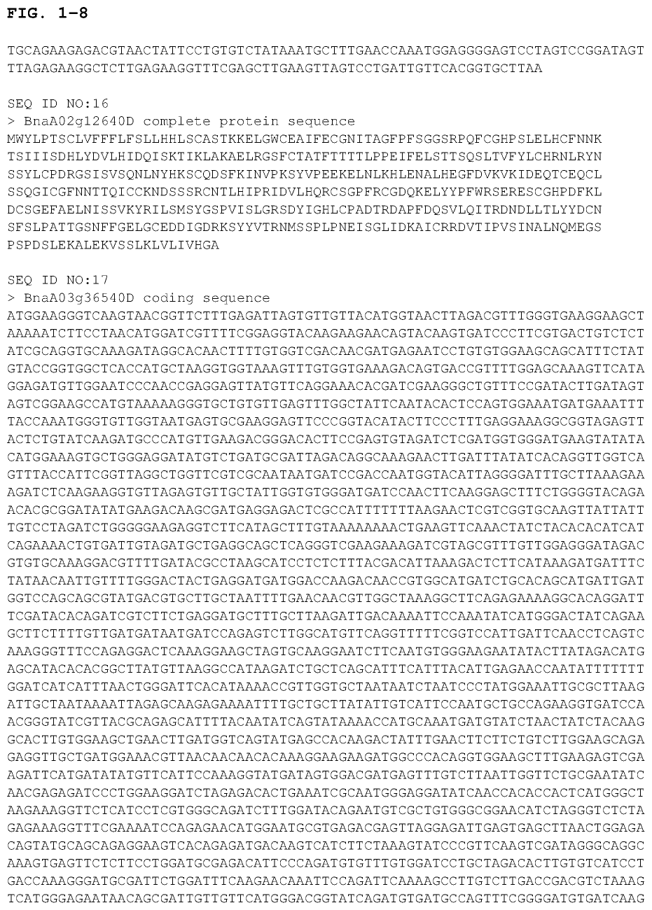

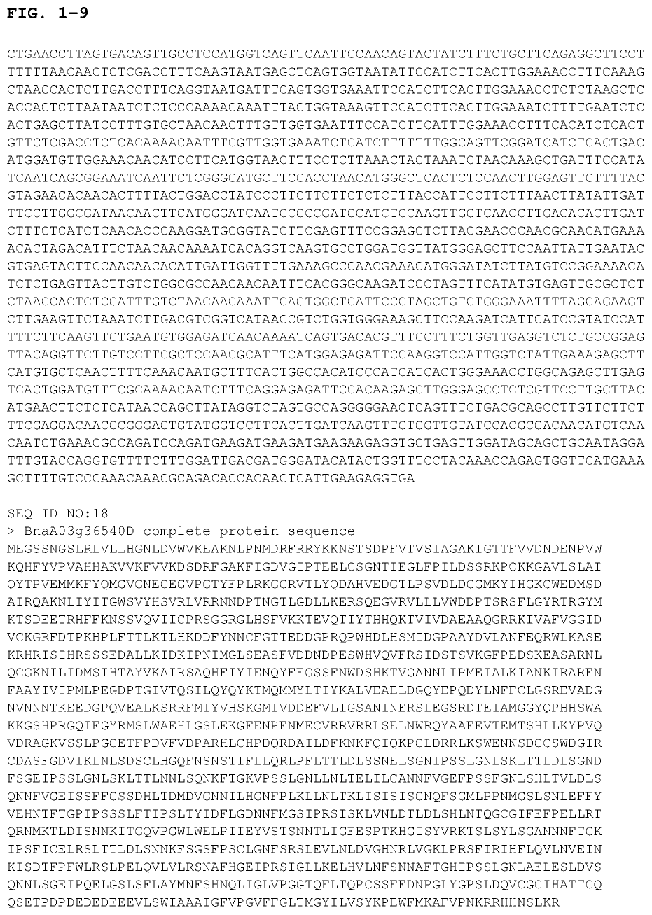

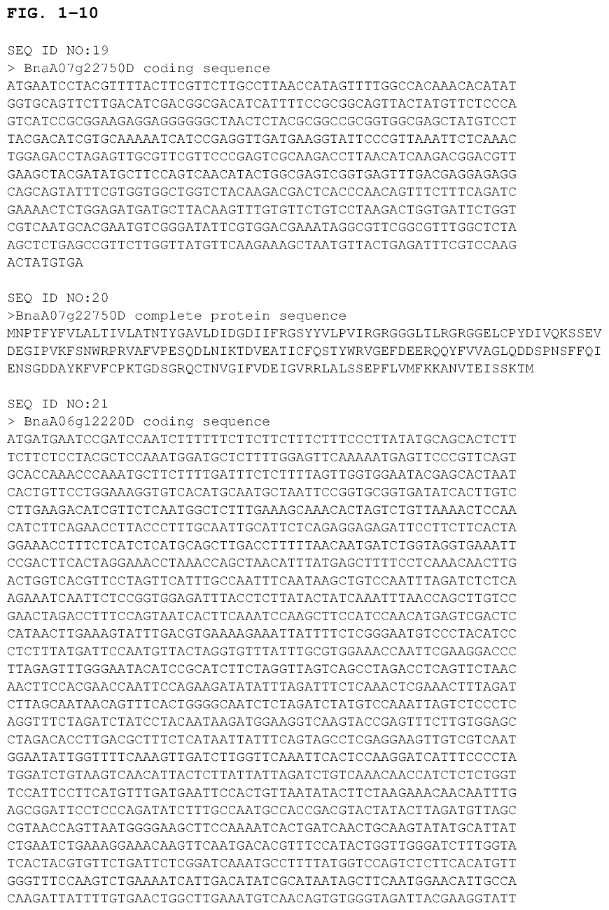

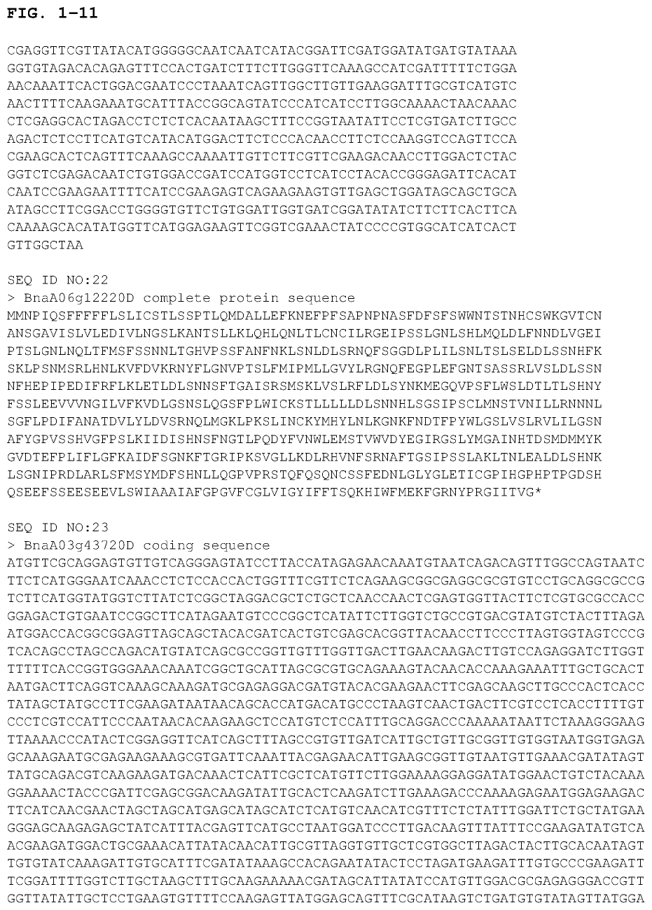

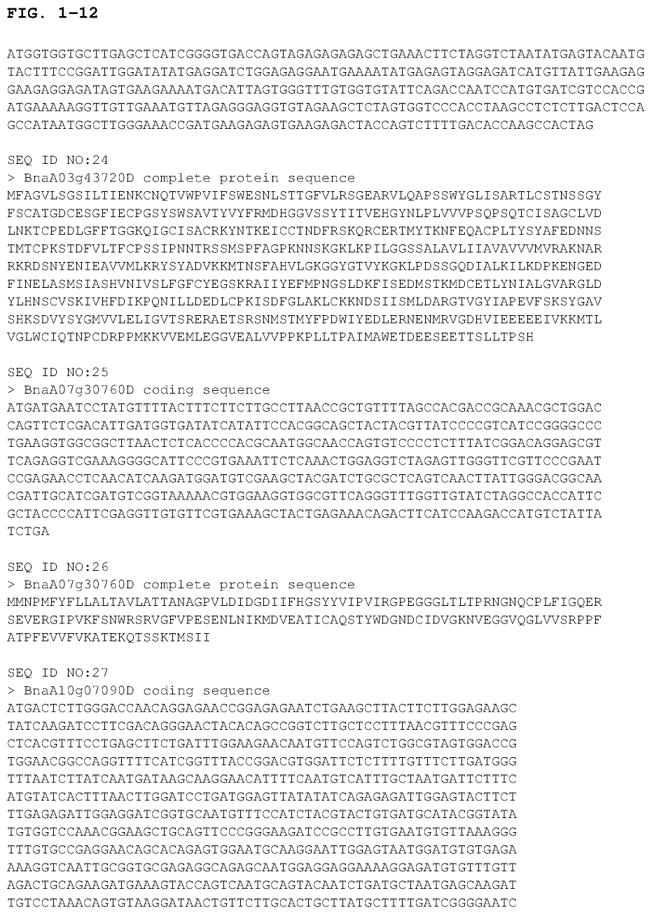

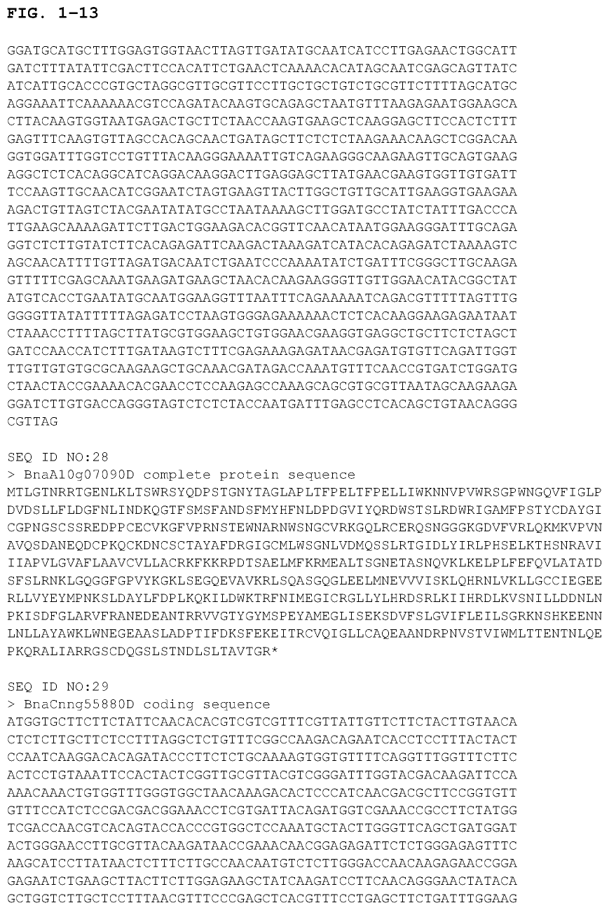

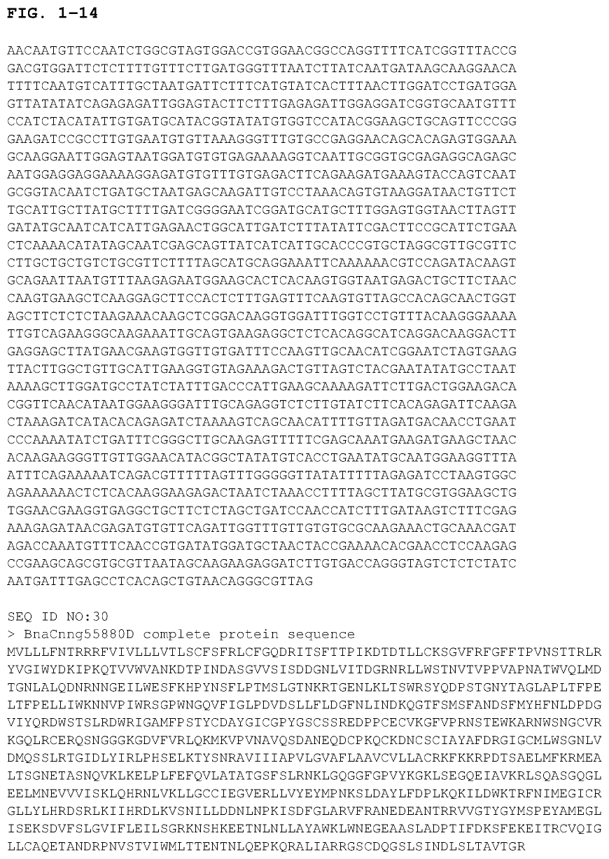

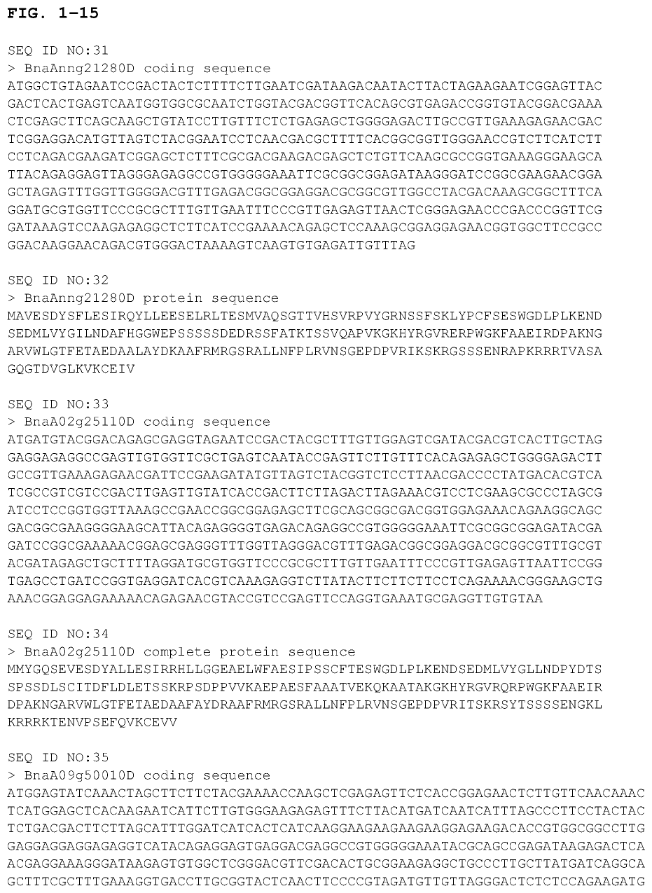

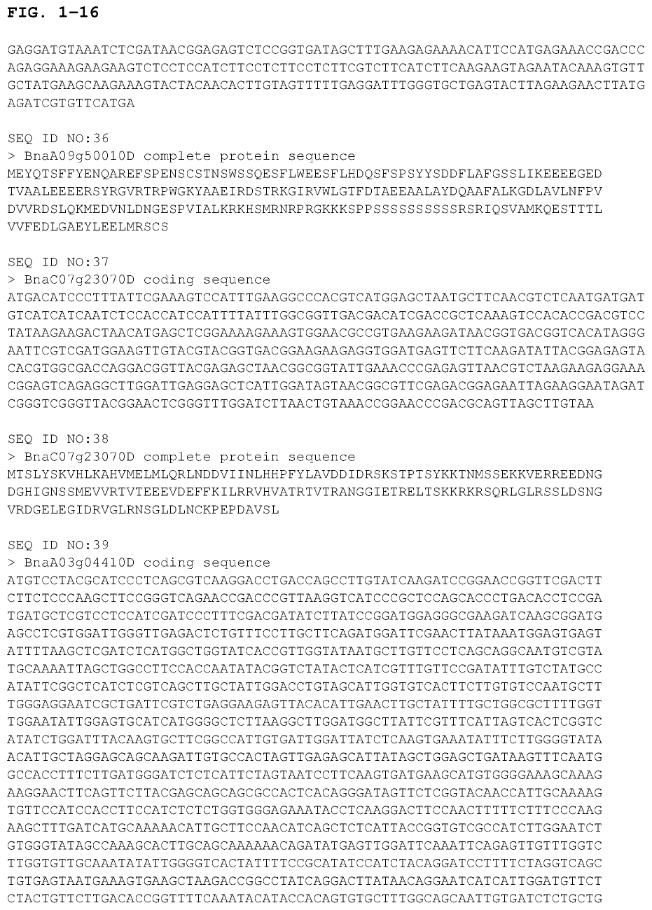

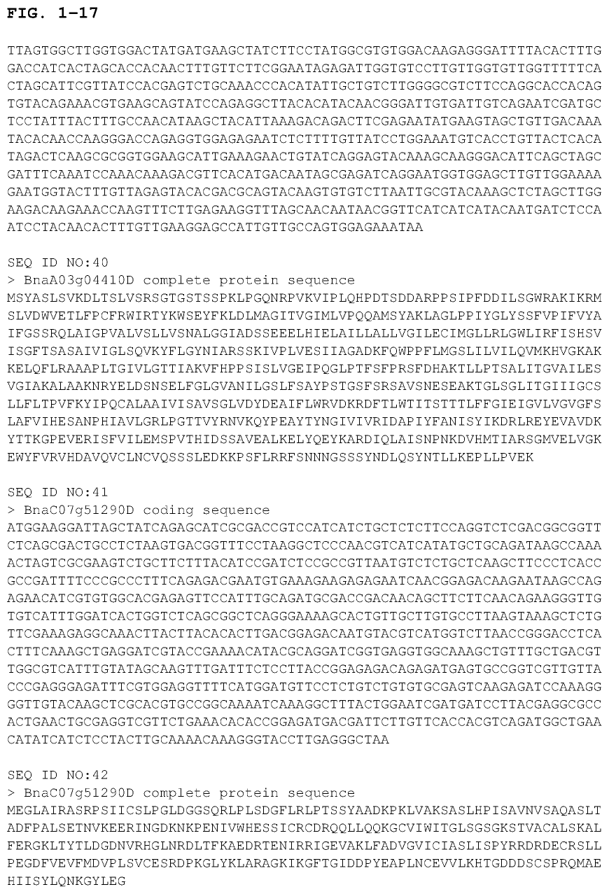

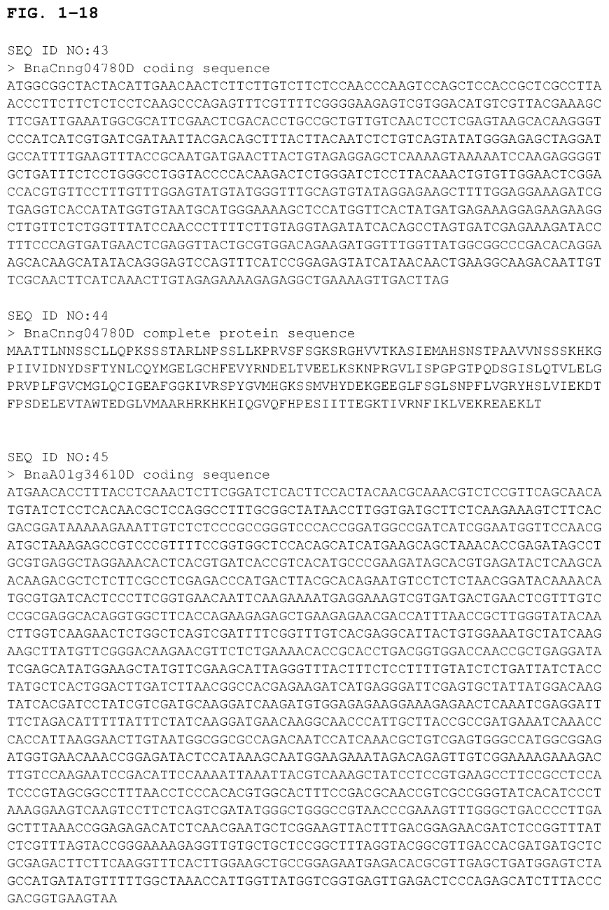

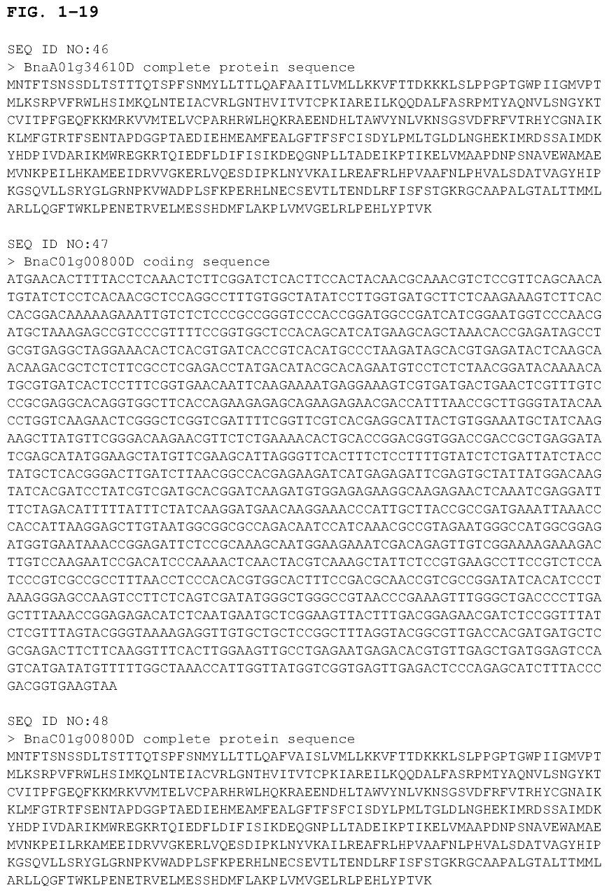

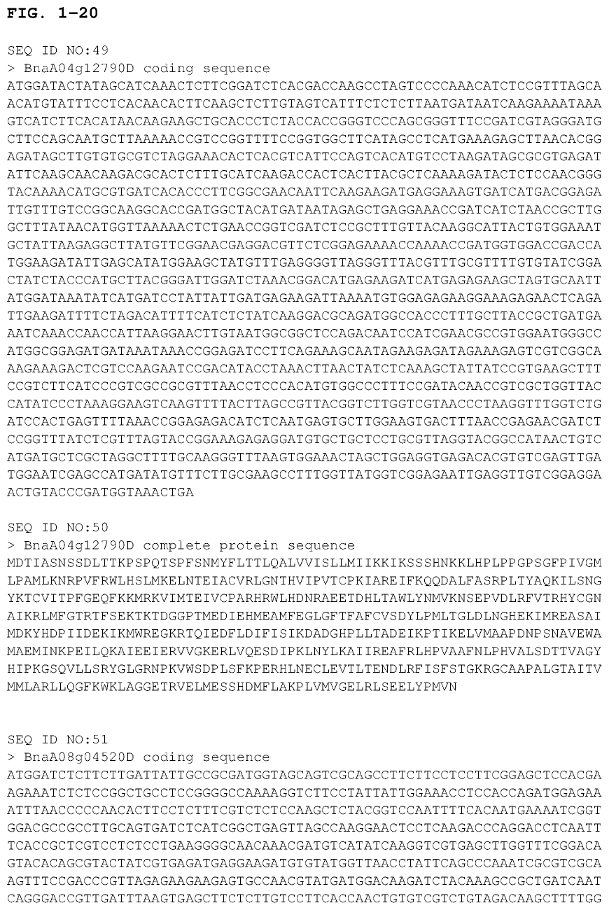

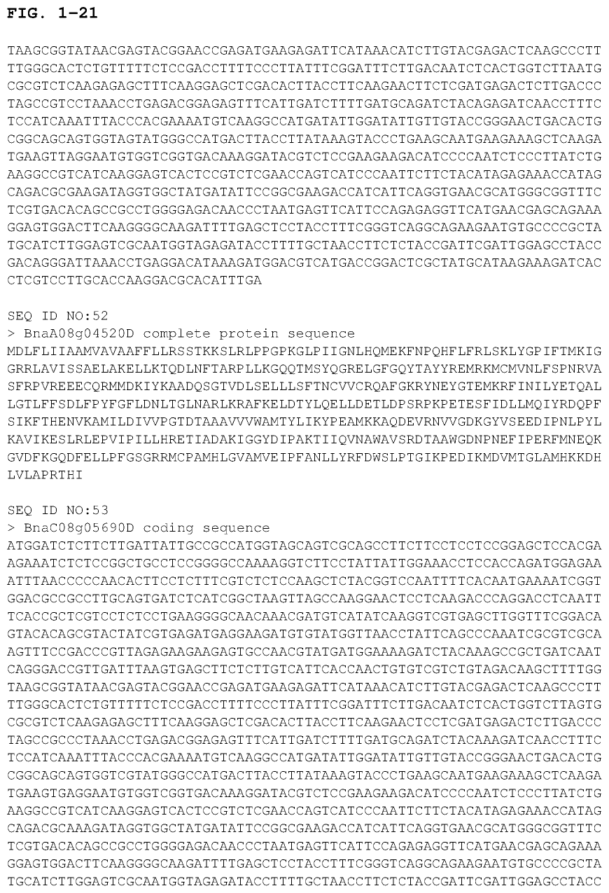

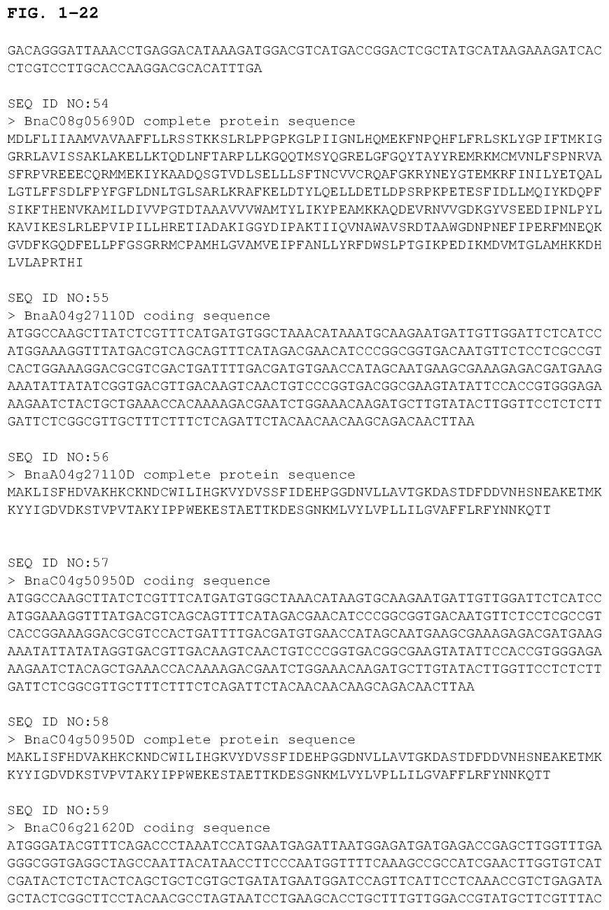

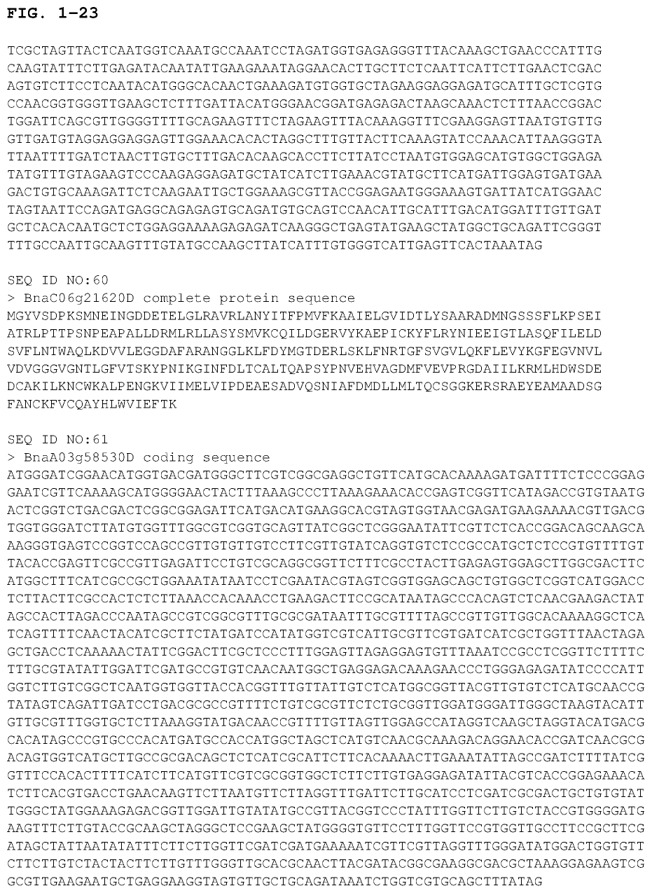

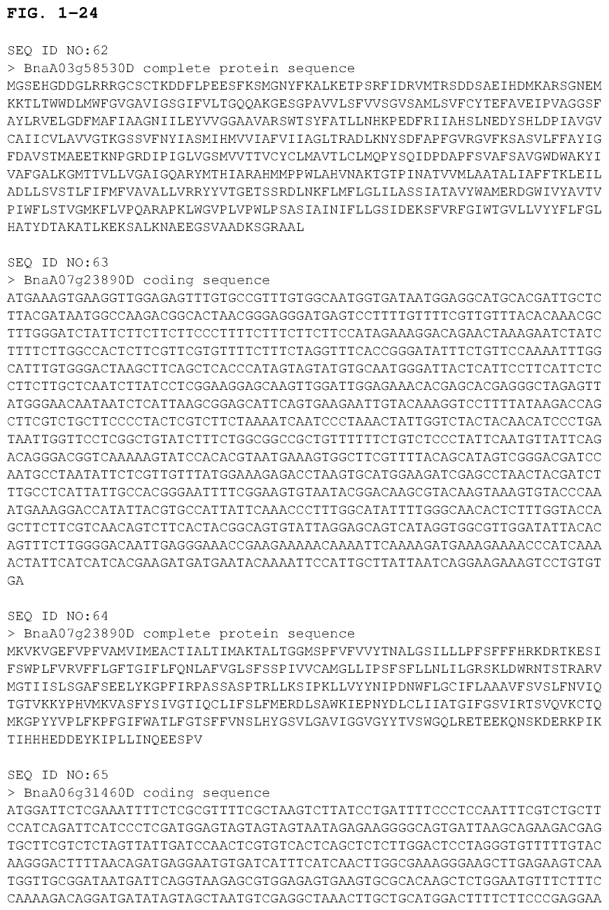

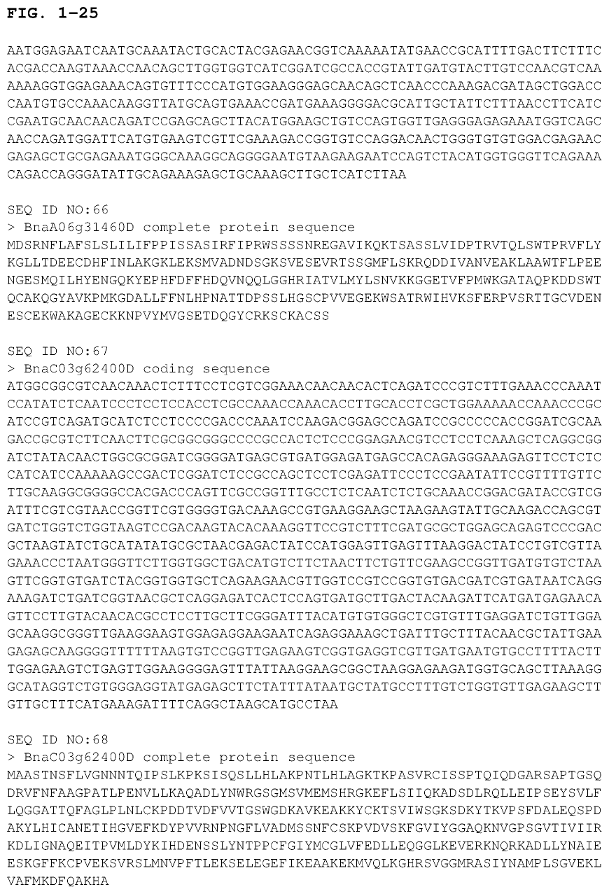

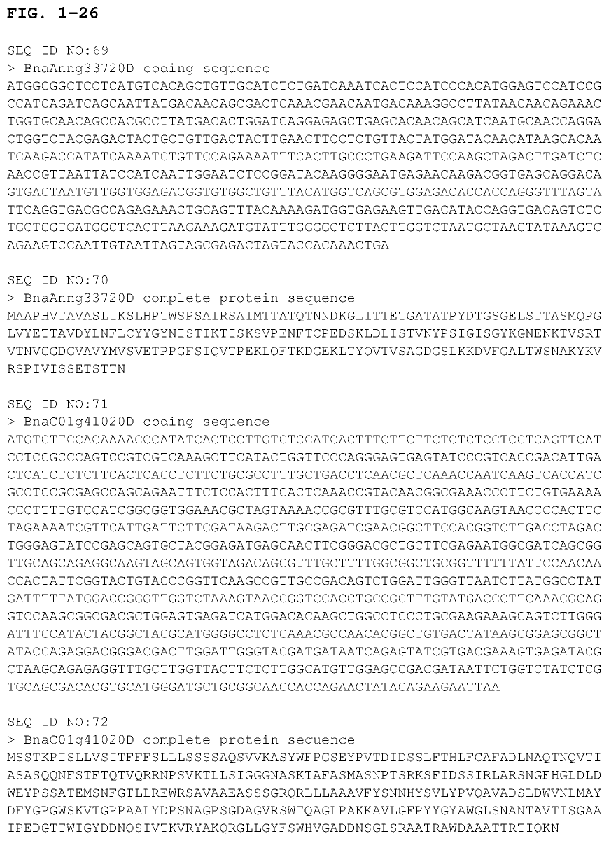

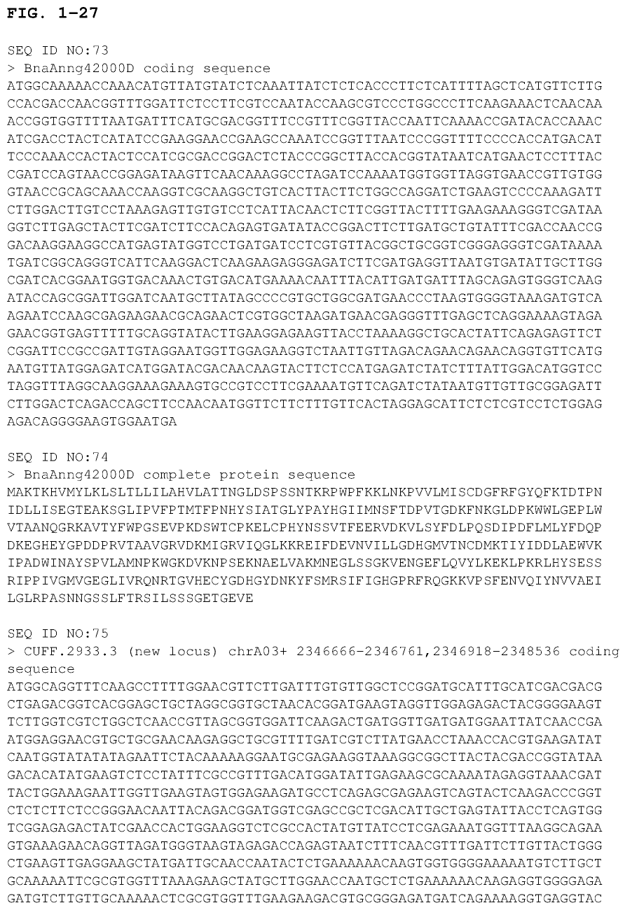

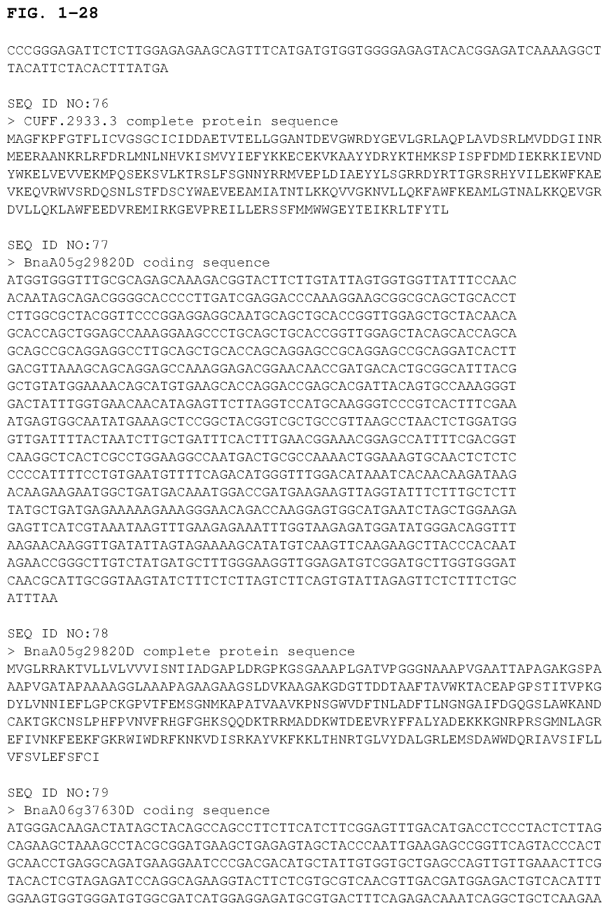

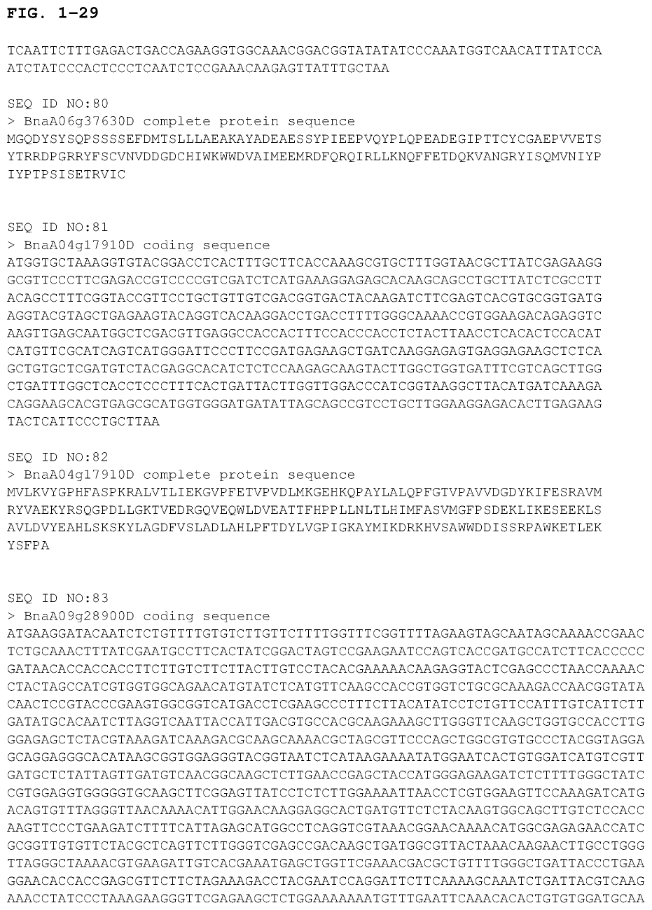

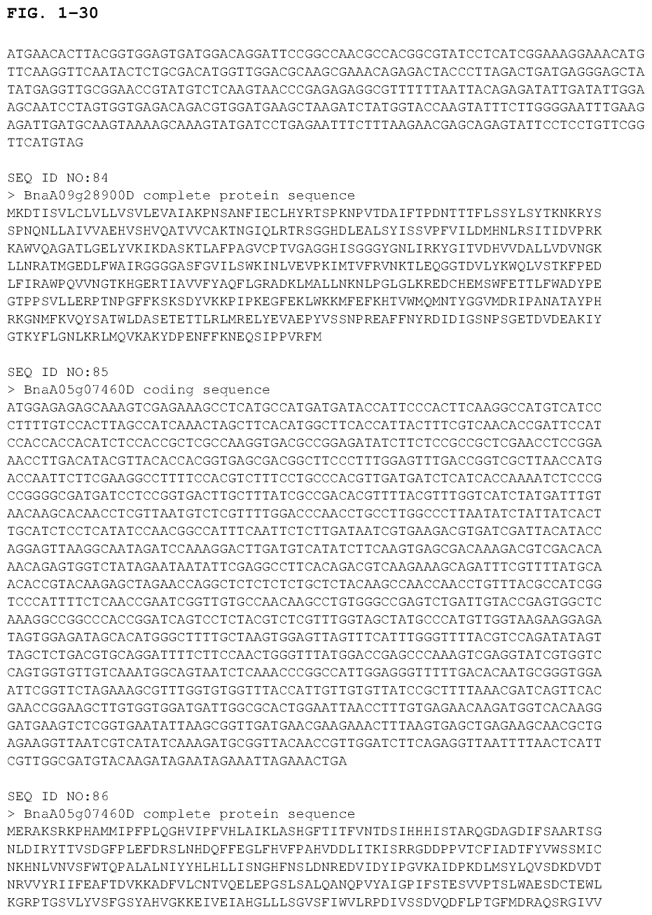

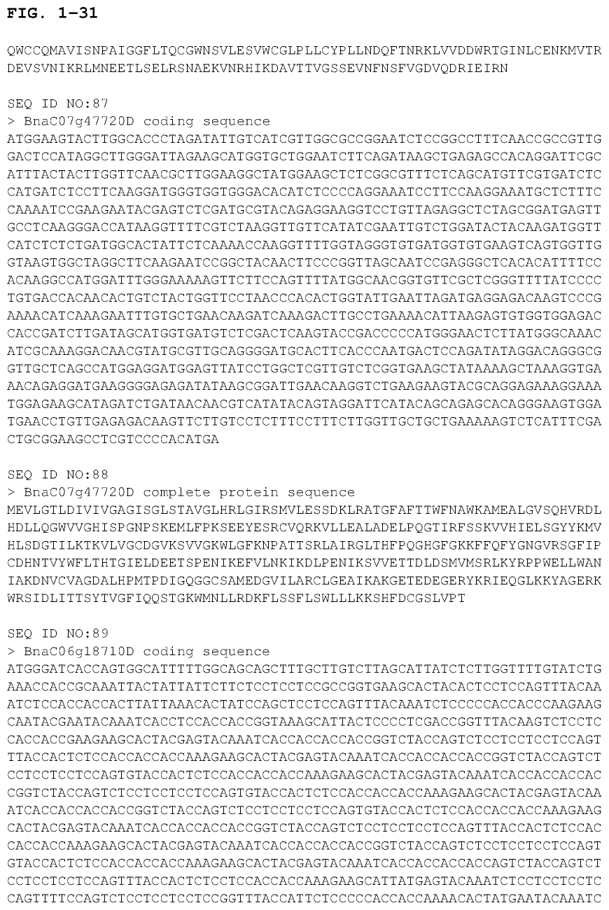

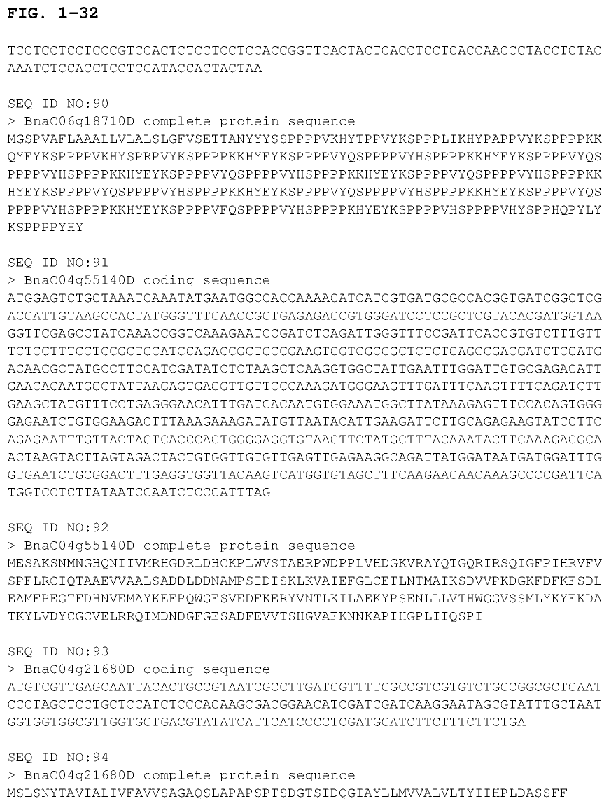

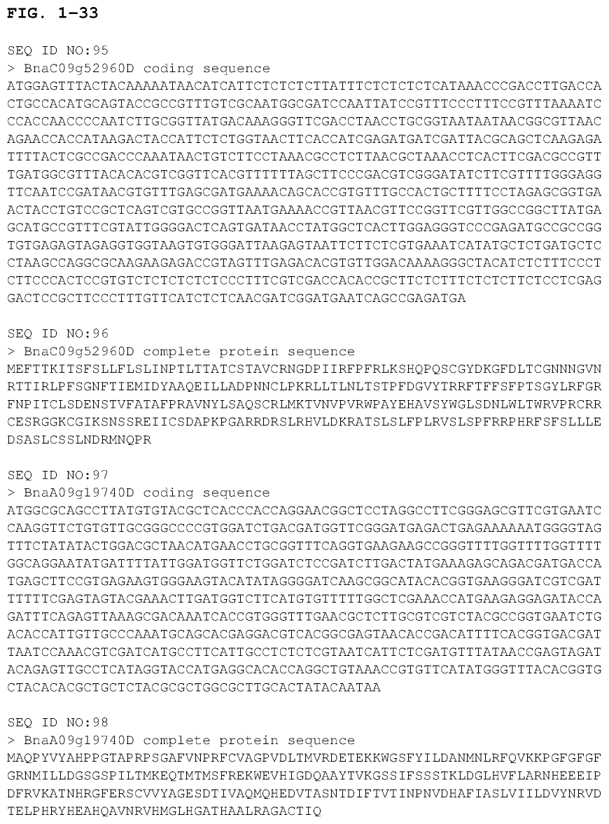

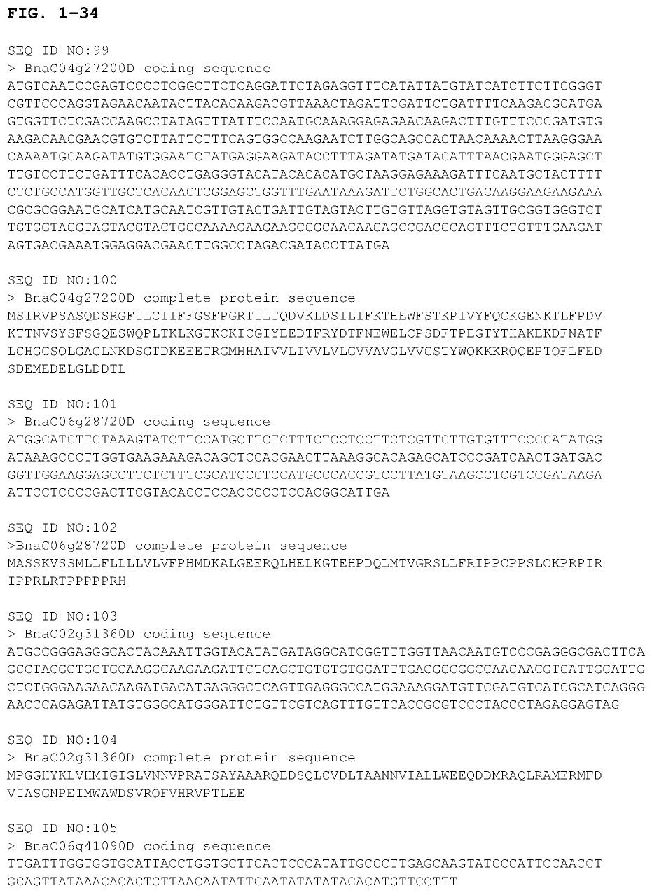

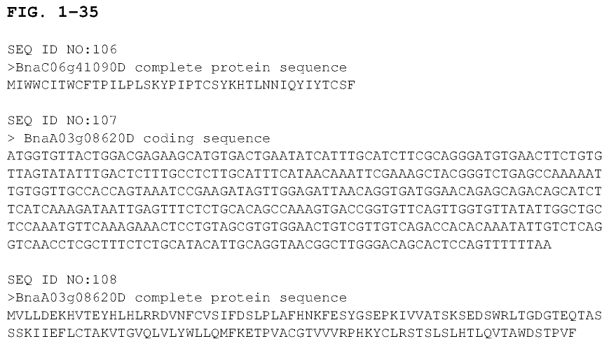

To investigate these genes further, Arabidopsis mutants containing a T-DNA insertion that disrupts gene activity were screened for defense response against L. maculans or the ability to form a lesion on the leaf surface. As Arabidopsis is naturally resistant to L. maculans (non host), a susceptible phenotype of a gene mutant suggested an essential function of that gene in pathogen detection and/or defense signaling. This analysis has identified several highly susceptible mutants, in which L. maculans asexual reproduction (pycnidia formation) is clearly visible. These genes are described in Table 5 of the Example, which includes the locus identifier for Brassica and for Arabidopsis. The coding regions and the proteins are shown in FIG. 1. No fungal reproduction was observed in leaves of wild-type or mock inoculated control plants. These data establish these genes as helpful for defense against L. maculans in Arabidopsis. Four of these have been expressed in a B. napus and increase resistance of the plant to the fungal pathogen. We expect that increased expression of one or more of the other 50 genes in plants susceptible to infection by L. maculans will increase resistance of the plant to this fungal pathogen.

Provided herein are isolated polynucleotides that include coding regions that increase resistance of a plant to infection by L. maculans, and isolated proteins encoded by the coding regions. Proteins useful herein and examples of polynucleotides encoding the proteins are described in FIG. 1. Also included are polynucleotides that include a coding region encoding a protein having at least one conservative substitution, polynucleotides that include a coding region with a nucleotide sequence having sequence similarity to a coding region depicted in FIG. 1, and proteins that are structurally similar to a protein described in FIG. 1.

A conservative substitution for an amino acid in a protein disclosed herein may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2.

Examples of polynucleotides that are coding regions are shown in FIG. 1. For instance, the coding region SEQ ID NO:1 encodes the protein SEQ ID NO:2, the coding region SEQ ID NO:3 encodes the protein SEQ ID NO:4, and so on. It should be understood that a polynucleotide encoding a protein described herein is not limited to one nucleotide sequence disclosed herein, but also includes the class of polynucleotides encoding the protein as a result of the degeneracy of the genetic code. For example, the nucleotide sequence SEQ ID NO:1 is but one member of the class of nucleotide sequences encoding a protein having the amino acid sequence SEQ ID NO:2, the nucleotide sequence SEQ ID NO:3 is but one member of the class of nucleotide sequences encoding a protein having the amino acid sequence SEQ ID NO:4, and so on. The class of nucleotide sequences encoding a selected protein sequence is large but finite, and the nucleotide sequence of each member of the class may be readily determined by one skilled in the art by reference to the standard genetic code, wherein different nucleotide triplets (codons) are known to encode the same amino acid.

Also included are polynucleotides that have sequence similarity to a coding region of FIG. 1. Whether a polynucleotide is structurally similar to a polynucleotide of FIG. 1 can be determined by aligning the residues of the two polynucleotides (for example, a candidate polynucleotide and any appropriate reference polynucleotide described herein) to optimize the number of identical nucleotides along the lengths of their sequences; gaps in either or both sequences are permitted in making the alignment in order to optimize the number of identical nucleotides, although the nucleotides in each sequence must nonetheless remain in their proper order. A reference polynucleotide may be a polynucleotide described herein. In one embodiment, a reference polynucleotide is a polynucleotide described in FIG. 1. A candidate polynucleotide is the polynucleotide being compared to the reference polynucleotide. A candidate polynucleotide may be isolated, for example, from a plant, or can be produced using recombinant techniques, or chemically or enzymatically synthesized. A candidate polynucleotide may be present in the genome of a plant and predicted to encode a protein useful herein.

A pair-wise comparison analysis of nucleotide sequences can be carried out using the Blastn program of the BLAST search algorithm, available through the World Wide Web, for instance at the internet site maintained by the National Center for Biotechnology Information, National Institutes of Health. Preferably, the default values for all Blastn search parameters are used. Alternatively, sequence similarity may be determined, for example, using sequence techniques such as GCG FastA (Genetics Computer Group, Madison, Wis.), MacVector 4.5 (Kodak/IBI software package) or other suitable sequencing programs or methods known in the art.

Thus, as used herein, a candidate polynucleotide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% nucleotide sequence identity to a reference nucleotide sequence.

Also provided herein are polynucleotides capable of hybridizing to a nucleotide sequence encoding a protein described herein. The hybridization conditions may be medium to high stringency. A maximum stringency hybridization can be used to identify or detect identical or near-identical polynucleotide sequences, while an intermediate or low stringency hybridization can be used to identify or detect polynucleotide sequence homologues.

Whether a protein is structurally similar to a protein of FIG. 1 can be determined by aligning the residues of the two proteins (for example, a candidate protein and any appropriate reference protein described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein. In one embodiment, a reference protein is a protein described in FIG. 1. A candidate protein is the protein being compared to the reference protein. A candidate protein can be isolated, for example, from a plant, or can be produced using recombinant techniques, or chemically or enzymatically synthesized.

Unless modified as otherwise described herein, a pair-wise comparison analysis of amino acid sequences can be carried out using the Blastp program of the Blastp suite-2sequences search algorithm, as described by Tatusova et al., (FEMS Microbiol Lett, 174, 247-250 (1999)), and available on the National Center for Biotechnology Information (NCBI) website. The default values for all blastp suite-2sequences search parameters may be used, including general paramters: expect threshold=10, word size=3, short queries=on; scoring parameters: matrix=BLOSUM62, gap costs=existence:11 extension:1, compositional adjustments=conditional compositional score matrix adjustment. Alternatively, proteins may be compared using other commercially available algorithms, such as the BESTFIT algorithm in the GCG package (version 10.2, Madison Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions.

Thus, as used herein, reference to an amino acid sequence disclosed in FIG. 1 can include a protein with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference amino acid sequence.

Alternatively, as used herein, reference to an amino acid sequence disclosed in FIG. 1 can include a protein with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

A protein that is structurally similar to a protein disclosed herein, for instance a protein of FIG. 1, has the biological activity of increasing resistance to infection by L. maculans. A protein described herein, therefore, can also be referred to as a resistance protein. Whether a structurally similar protein has biological activity can be determined by expressing the protein in a transgenic plant and comparing the transgenic plant to a control plant. Alternatively, the Arabidopsis-L. maculans model pathosystem can be used. the Arabidopsis-L. maculans model pathosystem is recognized in the art as correlating to the pathogenesis of L. maculans on plants of the genus Brassica (Bohman et al., The Plant Journal 37.1 (2004): 9-20; Staal et al., The Plant Journal 46.2 (2006): 218-230; Elliott et al., Molecular Plant 1.3 (2008): 471-481; and Petit et al., 29. Fungal genetics conference Asilomar 17. 2017). Methods for using the Arabidopsis-L. maculans model pathosystem are routine and known to the person of ordinary skill in the art (see also Example 1).

A coding region of an isolated polynucleotide described herein may be operably linked to a regulatory sequence. One example of a regulatory region is a promoter. A promoter is a polynucleotide that binds RNA polymerase and/or other transcription regulatory elements. A promoter facilitates or controls the transcription of DNA or RNA to generate an RNA molecule from a polynucleotide that is operably linked to the promoter. The RNA can be transcribed to yield a protein. Useful promoters useful include constitutive promoters, inducible promoters, and/or tissue preferred promoters for expression of a polynucleotide in a particular tissue or intracellular environment, examples of which are known to one of ordinary skill in the art. In one embodiment, a coding region is operably linked to a heterologous promoter.

A constitutive promoter refers to a promoter that is transcriptionally active during most, but not necessarily all, phases of growth and development and under most environmental conditions, in at least one cell, tissue or organ. Examples of useful constitutive plant promoters include, but are not limited to, the cauliflower mosaic virus (CaMV) 35S promoter, (Odel et al., 1985, Nature, 313:810), the nopaline synthase promoter (An et al., 1988, Plant Physiol., 88:547), and the octopine synthase promoter (Fromm et al., 1989, Plant Cell 1: 977).

An inducible promoter has induced or increased transcription initiation in response to a chemical, environmental, or physical stimulus. Examples of inducible promoters include, but are not limited to, auxin-inducible promoters (Baumann et al., 1999, Plant Cell, 11:323-334), cytokinin-inducible promoters (Guevara-Garcia, 1998, Plant Mol. Biol., 38:743-753), and gibberellin-responsive promoters (Shi et al., 1998, Plant Mol. Biol., 38:1053-1060). Additionally, promoters responsive to heat, light, wounding, pathogen resistance, and chemicals such as methyl jasmonate or salicylic acid, can be used, as can tissue or cell-type specific promoters such as xylem-specific promoters (Lu et al., 2003, Plant Growth Regulation 41:279-286).

A tissue preferred promoter is one that is capable of preferentially initiating transcription in certain organs or tissues, such as the leaves, roots, seed tissue etc. For example, a root-specific promoter is a promoter that is transcriptionally active predominantly in plant roots, substantially to the exclusion of any other parts of a plant, while still allowing for any leaky expression in these other plant parts. Promoters able to initiate transcription in certain cells only are referred to herein as cell-specific.

A seed-specific promoter is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters (endosperm/aleurone/embryo specific) are described in Russinova and Reuzeau (US Patent Application 20120331584). Further examples of seed-specific promoters are given in Qing Qu and Takaiwa (2004, Plant Biotechnol. J., 2:113-125). A green tissue-specific promoter is a promoter that is transcriptionally active predominantly in green tissue, substantially to the exclusion of any other parts of a plant, while still allowing for any leaky expression in these other plant parts.

Another example of a tissue-specific promoter is a meristem-specific promoter, which is transcriptionally active predominantly in meristematic tissue, substantially to the exclusion of any other parts of a plant, while still allowing for any leaky expression in these other plant parts. A further example of a tissue-specific promoter is the RuBisCo promoter, which is transcriptionally active predominantly in the leaf or cotyledon.

Other examples of promoters include, but are not limited to ubiquitin promoters and the native promoters and regulatory sequences operably linked to the coding regions of FIG. 1.

Another example of a regulatory region is a transcription terminator. Suitable transcription terminators are known in the art and include, for instance, a stretch of 5 consecutive thymidine nucleotides.

A polynucleotide may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. A vector may result in integration into a cell's genomic DNA. A vector may be capable of replication in a bacterial host, for instance E. coli or Agrobacterium tumefaciens. In one embodiment, the vector is a plasmid. Selection of a vector depends upon a variety of desired characteristics in the resulting construct, such as a selection marker, vector replication rate, and the like. Suitable host cells for cloning or expressing the vectors herein are prokaryotic or eukaryotic cells. Suitable eukaryotic cells include plant cells. Suitable prokaryotic cells include eubacteria, such as gram-negative organisms, for example, E. coli or A. tumefaciens.

A selection marker is useful in identifying and selecting a transformed cell or plant. Examples of such markers include, but are not limited to, a neomycin phosphotransferase (NPTII) gene (Potrykus et al., 1985, Mol. Gen. Genet., 199:183-188), which confers kanamycin resistance, a hygromycin B phosphotransfease (HPTII) gene (Kaster, et al, 1983, Nuc. Acid. Res. 19: 6895-6911), and a bialaphos acetyltransferase (bar) gene, conferring resistance to bialaphos (Richards et al., 2001, Plant Cell Rep. 20, 48-54, and Somleva et al., 2002, Crop Sci. 42, 2080-2087). Cells expressing the NPTII gene can be selected using an appropriate antibiotic such as kanamycin or G418. The HPTII gene encodes a hygromycin-B 4-O-kinase that confers hygromycin B resistance. Cells expressing HPTII gene can be selected using the antibiotic of hygromycin B (Kaster, et al, 1983, Nuc. Acid. Res. 19: 6895-6911, Blochlinger and Diggelmann, 1984, Mol. Cell. Biol. 4 (12): 2929-2931). Other commonly used selectable markers include a mutant EPSP synthase gene (Hinchee et al., 1988, Bio/Technology 6:915-922), which confers glyphosate resistance; and a mutant acetolactate synthase gene (ALS), which confers imidazolinone or sulphonylurea resistance (Conner and Santino, 1985, European Patent Application 154,204).

Polynucleotides described herein can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are well known. Methods for in vitro synthesis also include, for instance, in vitro transcription using a circular or linear expression vector in a cell free system. Expression vectors can also be used to produce a polynucleotide described herein in a cell, and the polynucleotide may then be isolated from the cell.

Provided in the present description are transgenic plants and host cells having increased expression of a coding region described herein and increased expression of the protein encoded by the coding region. A host cell includes the cell into which a coding region described herein was introduced, and its progeny. Accordingly, a host cell can be an individual cell, a cell culture, or cells that are part of an organism, e.g., a plant. The host cell can also be a portion of a leaf, a stem, a flower, an ovary, a fruit, or a callus. In one embodiment, the host cell is a plant cell. A host cell may be may be homozygous or heterozygous for a coding region encoding a protein described herein.

A host cell or a transgenic plant having increased expression of a coding region described herein may have an increased amount of mRNA encoding a protein, may have an increased amount of the protein, or a combination thereof, compared to a control, e.g., a control plant or a control host cell. The increase in the amount of an mRNA or a protein encoded by a coding region may be increased by at least 0.1%, at least 1%, at least 5%, at least 10%, at least 20%, at least 30%, at least 40%, at least 50%, at least 60%, at least 70%, at least 80%, or at least 90% compared to the amount of the mRNA or the amount of the protein in a control plant or control host cell.

Also provided is the plant material (such as, for instance, a stem, a branch, a root, a leaf, seed, a fruit, oil including oil from a seed, etc.) derived from a plant described herein.

Methods for increasing resistance of a plant to infection by L. maculans include, but are not limited to, increasing expression of an endogenous coding region to yield increased amounts of a protein in a plant, and increasing the copy number of a coding region in a plant. Increasing expression of a coding region in a plant may occur by introducing into a plant a recombinant coding region or by increasing expression of a native coding region in the plant. In another embodiment, increasing expression of a coding region in a plant may occur by introducing into a plant cell a recombinant coding region or by increasing expression of a native coding region in the plant cell, and then using routine methods to develop a transgenic plant from the plant cell. For instance, over-expression can be accomplished by introducing an exogenous promoter into a cell to drive expression of a coding region residing in the genome. Regulatory elements, such as promoters or enhancer elements, may be introduced in an appropriate position (typically upstream) of a coding region present in the genome of a plant to upregulate expression of the coding region. For example, endogenous promoters may be altered in vivo by mutation, deletion, and/or substitution (see, Kmiec, U.S. Pat. No. 5,565,350; Zarling et al., WO 93/22443), or promoters may be introduced into a plant cell in the proper orientation and distance from a coding region encoding a protein disclosed herein to control the expression of the gene. The effect of over-expression of a given coding region on the phenotype of a plant can be evaluated by comparing plants over-expressing the coding region to control plants.

Transformation of a plant with a polynucleotide described herein to result in increased expression of a coding region and increased amounts of a protein results in the phenotype of increased resistance to infection by L. maculans. Whether a transgenic plant has altered resistance to infection by L. maculans can be determined by comparing resistance of the transgenic plant and a control plant. Increased resistance of a plant to infection by L. maculans refers to a reduction in damage caused by L. maculans infection compared to damage caused on a control plant. Damage caused by L. maculans is known to the person of ordinary skill in the art and includes, but is not limited to, leaf symptoms, stem symptoms, and loss of yield. Accordingly, damage can be assessed by number and size of leaf symptoms, frequency and severity of stem symptoms, and lodging of plants due to stem infection.

Increased resistance can be due to, for instance, reduction or prevention of infection, reproduction, spread, or survival of L. maculans in a plant. In one embodiment, reduced reproduction may be decreased asexual reproduction, such as reduced pycnidia formation. Increased resistance also includes a plant that is completely resistant, for instance, a plant on which no disease symptoms are found. Increased resistance of a plant can be carried out in controlled environments, such as growth chambers, or in field trials.

A plant with increased resistance to L. maculans is a member of the genus Brassica (referred to herein as Brassica sp.), such as B. napus, B. oleraceae, B. rapa, B. juncea, B. balearica, B. carinata, B. elongate, B. fruticulosa, B. hilarionis, B. narinosa, B. nigra, B. perviridis, B. rupestris, B. septiceps, or B. tournefortii.

Transgenic plants described herein may be produced using routine methods (see, for instance, Waterhouse et al., US Patent Application 2006/0272049). Methods for transformation and regeneration are known to the skilled person. Transformation of a plant cell with a polynucleotide described herein to yield a recombinant host cell may be achieved by any known method for the insertion of a polynucleotide into a prokaryotic or eukaryotic host cell, including Agrobacterium-mediated transformation protocols, viral infection, whiskers, electroporation, microinjection, polyethylene glycol-treatment, heat shock, lipofection, particle bombardment, and chloroplast transformation.

In one embodiment, a coding region described herein may be used to make a transgenic plant, such as a transgenic B. napus. In other embodiments, a coding region that is a homologue of a coding region shown in FIG. 1 may be used with other members of the genus Brassica. Coding regions that are homologues are coding regions that share ancestry, e.g., they are both derived from a coding region present in a common ancestor. The skilled person can easily determine if a coding region in a non-B. napus plant is a homolog of a coding region disclosed herein through the use of routine methods. In one embodiment, the skilled person can use the nucleotide sequence of a coding region disclosed herein and design degenerate PCR primers for use in a low stringency PCR. Low stringency PCR is a routine method for identifying homologs of known coding region. In another embodiment, the skilled person can use readily available databases to identify in another member of the genus Brassica a homolog of a coding region disclosed herein.

In another embodiment, the skilled person can identify a homolog of a coding region disclosed herein by the level of sequence identity between the coding region disclosed herein and another coding region. In one embodiment, when two nucleotide sequences are being compared, percent identities greater than 50% are taken as evidence of possible homology. The E value (Expect value) indicates the number of hits (sequences) in the database searched that are expected to align to the query simply by chance, so a E value less than 0.01 (i.e., less than 1% chance of the sequence occurring randomly), coupled with a percent identity of greater than 50% is considered a suitable score to identify a probable homolog. Methods for determining nucleotide sequence identity between two sequences are readily available and routine in the art. In one embodiment, coding regions in a member of the genus Brassica that are homologues of the coding regions in FIG. 1 may be identified using the BLAST-X algorithm against the non-redundant database at NCBI with default parameters. A candidate coding region is considered to be a homologue of a coding region disclosed in FIG. 1 if the candidate coding region has at least greater than 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% nucleotide sequence identity to the respective coding regions in FIG. 1.

The cells that have been transformed may be grown into plants in accordance with conventional techniques. See, for example, McCormick et al. (1986, Plant Cell Reports, 5:81-84). These plants may then be grown and evaluated for expression of the coding region. These plants may be either pollinated with the same transformed strain or different strains, and the resulting hybrid having desired phenotypic characteristics identified. Breeding procedures such as crossing, selfing, and backcrossing are known in the art. Two or more generations may be grown to ensure that the desired expression of one or more coding regions is stably maintained and inherited and then seeds harvested to ensure stability of the desired characteristics have been achieved.

Also provided by the present disclosure are methods. In one embodiment, a method includes producing oil. The method includes harvesting seeds from a transgenic plant or a part thereof and extracting the oil from the seeds.

In one embodiment, a method includes preparing a food, a feed, or an industrial product. A food refers to a use for human diet, and a feed refers to a use for a non-human animal diet. The method includes obtaining a transgenic plant or a part thereof, and preparing the food, feed or industrial product from the plant or part thereof. Examples of food or feed include, but are not limited to, oil, meal, grain, starch, flour, or protein. Examples of an industrial product include, but is not limited to, biofuel, fiber, and industrial chemical, a pharmaceutical or a nutraceutical.

In another embodiment, a method includes making an oil, such as a canola oil. Harvested canola seed can be crushed to extract crude oil and, if desired, refined, bleached and deodorized by techniques known in the art.

In another aspect, provided herein is a kit. In some embodiments, the kit includes a seed from a transgenic plant as described herein. In some embodiments, the kit includes a vector as described herein. In some embodiments, each of the materials and reagents required for introducing a vector into a plant or host cell can be assembled together in a kit. The components of a kit including a vector may be provided in an aqueous form or a dried or lyophilized form. The kit may include an instruction sheet defining introducing the vector into a plant or host cell, or defining conditions for planting the seed.

Also, in the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLE 1

The hemibiotrophic fungal pathogen Leptosphaeria maculans is the causal agent of blackleg disease in Brassica napus (canola, oilseed rape) and causes significant yield loss worldwide. While genetic resistance has been used to mitigate the disease using traditional breeding strategies, there is little knowledge about the genes contributing to blackleg resistance. RNA sequencing and a streamlined bioinformatics pipeline identified genes and plant defense pathways specific to plant resistance in the B. napus-L. maculans LepR1-AvrLepR1 interaction over time. The temporal analyses were complemented by monitoring gene activity directly at the infection site using laser microdissection coupled to qPCR. Finally, the genes involved in plant resistance to blackleg in the Arabidopsis-L. maculans model pathosystem were characterized. Data reveal an accelerated activation of the plant transcriptome in resistant host cotyledons associated with transcripts coding for extracellular receptors and phytohormone signaling molecules. Functional characterization provides direct support for transcriptome data and positively identifies resistance regulators in the Brassicaceae. Spatial gradients of gene activity were identified in response to L. maculans proximal to the site of infection. This dataset provides unprecedented spatial and temporal resolution into the genes required for blackleg resistance and serves as a valuable resource to those interested in host-pathogen interactions.

Little is known about the downstream biological processes underlying Brassica napus resistance against Leptosphaeria maculans, the causal agent of blackleg disease. Detailed transcriptome analysis of the B. napus defense response to L. maculans provides new insights into blackleg resistance mechanisms and identifies genes that can be targeted for crop improvement and protection.

Brassica napus ranks second in production among oilseed crops worldwide and is under constant threat of blackleg disease caused by the hemibiotrophic fungal pathogen, Leptosphaeria maculans (Fitt et al., 2006). Currently, mitigation of crop loss relies largely on race-specific resistance (R) genes and their corresponding pathogen avirulence (Avr) genes (Larkan et al., 2015). Interaction between the products of R and Avr results in an incompatible host-pathogen interaction and pathogen restriction from host tissues. Absence of either the R- or Avr- gene results in a compatible host-pathogen interaction and host colonization. Each interaction is likely governed by large sets of genes activated over time and under the control of cellular receptors and signal transduction cascades that determine host fate. Although R-genes conferring blackleg resistance have been identified in canola (Marcroft et al., 2012; Larkan et al., 2013), it is unclear by which mechanisms these genes effectively inhibit L. maculans colonization. Previous transcriptome studies of the B. napus-L. maculans pathosystem limit analyses to compatible interactions and focus on pathogen virulence and effectors (Lowe et al., 2014; Haddadi et al., 2016). Thus, there is a need to identify the genes facilitating host resistance against L. maculans and define how the host defense response is controlled in both space and time.

Plant defense response mechanisms are commonly subdivided into two immune pathways: pattern triggered immunity (PTI) and effector triggered immunity (ETI) (Jones and Dangl, 2006; Thomma et al., 2011). PTI is characterized by the detection of pathogen associated molecular patterns (PAMPs) via extracellular membrane receptors such as receptor-like proteins (RLPs) and receptor-like kinases (RLKs), while ETI is characterized by the detection of pathogen effectors or their perturbation of host molecules by intracellular nucleotide binding-leucine rich repeat (NB-LRR) receptors (Tsuda and Katagiri, 2010; Dangl et al., 2013). Both immune pathways share cellular machinery to elicit a defense response; however, PTI is associated with non-host resistance, and ETI (in conjunction with PTI) with host incompatibility (Bigeard et al., 2015). Although a useful model, this ETI/PTI dichotomy cannot be effectively applied to the Arabidopsis- or B. napus-L. maculans pathosystems. Not only are effector-triggered NB-LRR receptors required for Arabidopsis non-host resistance to L. maculans (Staal et al., 2006), but the recently cloned B. napus R-gene, LepR3, has been identified as a transmembrane RLP (Larkan et al., 2013). Thus, effector triggered defense (ETD) was proposed by Stotz et al. (2014) and refers specifically to RLP-triggered incompatible interactions. Unlike the rapid cell death observed in ETI, ETD is often associated with a delayed onset of cell death, as observed in B. napus-L. maculans incompatible interactions (Stotz et al., 2014). As L. maculans grows apoplastically, the ability of R-gene products to detect pathogens in the extracellular space is logical and supports the ETD paradigm.

Following the recognition of hemibiotrophic pathogens, early defense responses such as the activation of mitogen-activated protein kinases (MAPKs) are triggered within the cell (Meng and Zhang, 2013). Subsequently, large-scale transcriptional reprogramming contributes to the regulation of phytohormone signaling pathways (Denancé et al., 2013). Jasmonic acid (JA) and abscisic acid (ABA) are both involved in Arabidopsis non-host resistance to L. maculans (Kaliff et al., 2007), and JA, ethylene (ET) and salicylic acid (SA) signaling pathways are activated during the B. napus-L. maculans host-incompatible interaction (Sas̆ek et al., 2012). Although hormone signaling has been described temporally across the plant defense response to fungal pathogens (reviewed in Mishra et al., 2012), there are no data on the spatial partitioning of these genes following ETD in host tissues.

Downstream plant defense responses in hemibiotrophic pathosystems may involve the deposition of callose (Ellinger et al., 2013). Callose deposition is typically triggered by PAMPs, and PAMP-induced callose deposition has been used as a marker for PTI activity in Arabidopsis (Luna et al., 2011). Indole glucosinolates (IGS), bioactive secondary metabolites with anti-fungal capabilities, also promote production of callose (Clay et al., 2009). In Arabidopsis, resistance to hemibiotrophic fungi can be dependent on the production of IGS (Hiruma et al., 2013) or callose deposition (Staal et al., 2006; Kaliff et al., 2007), however their role in the Brassica napus-L. maculans pathosystem remains unclear.

The transcriptome of B. napus cotyledons inoculated with L. maculans across a two-week infection period was profiled to explore the activation of ETD pathways and identify specific regulators and genes contributing to host resistance. Detailed anatomical observations complement the molecular analyses and clearly show the delayed onset of cell death indicative of ETD. Genes activated exclusively in resistant cotyledons were disrupted in Arabidopsis and positively identify uncharacterized receptors, negative cell death regulators, and activators of sulfur metabolism that contribute to L. maculans defense in the Brassicaceae. We explored the activity of these genes and defense markers directly at, and proximal to, the infection site. Data show tightly-controlled spatial transcriptional gradients developed during ETD that are associated with pathogen detection, IGS production, and hormone signaling. Taken together, these data provide a global transcriptome analysis of ETD against L. maculans and show early activation of defense pathways in resistant cotyledons that are controlled in space and time.

### EXPERIMENTAL PROCEDURE

### Plant and Fungal Materials.

Susceptible B. napus cultivar Westar and B. napus line DF78 (Rlm3, LepR1) were inoculated with L. maculans isolate D3 (AvrLm5, AvrLepR1; Zhang et. al., 2016). Canola seedlings were grown in controlled environments with a 16-h photoperiod (16° C. dark, 21° C. light). Plants were grown in Sunshine mix #4 (SunGro Horticulture, available on the world wide web at sungro.com). Fungal inoculum was prepared according to Zhang et al., (2015). Seven day old seedlings were point-inoculated with 10 μL of D3 pycnidiospore suspension (2×107 pycnidiospores mL-1) or sterilized distilled water (mock).

### Microscopy, Lignin and Callose Deposition.

Cotyledons were processed for light microscopy exactly as reported in Chan and Belmonte (2013) using the Leica Historesin embedding procedure (Leica Microsystems). Sections cut 3 μm thick were stained with periodic acid-Schiff's (PAS) and counterstained with toluidine blue 0 (TBO) for general structure. For trypan blue/aniline blue staining of fungal hyphae, fresh canola cotyledons were cleared in acetic acid: ethanol (1: 3, v/v) and stained with 0.01% trypan blue or 0.05% aniline blue in lactoglycerol (lactic acid: glycerol: dH2O=1:1:1, v/v/v). To visualize plant lignified materials, canola cotyledons were cleared in 95% ethanol and stained in phloroglucinol-HCl (a saturated solution of Phloroglucinol in 20% HCl). Callose deposition was visualized using aniline blue staining. Cotyledons were incubated in K₂HPO₄ buffer for 30 mins and incubated in 0.05% aniline blue using fluorescence microscopy (near UV, 395 nm). All sections and tissues were visualized on a Zeiss Axio Imager Z1. Scanning electron micrographs were captured using the Hitachi T-1000, to examine fungal infection on the surface of freshly collected canola cotyledons without tissue fixation.

### Construction of RNA Sequencing Libraries.

RNA was collected from three biological replicates of infected- and two mock inoculated B. napus cotyledons at 0, 3, 7, and 11 dpi. Total RNA was isolated by using PureLink® Plant RNA Reagent (Ambion) and treated with TURBO DNA-free™ Kit (Ambion) according to the manufacturer's instructions. RNA quality and integrity was measured using the 2100 Bioanalyzer (Agilent Technologies) with the Agilent 2100 PicoChip. RNA-sequencing libraries were prepared according to alternative HTR protocol (C2) developed by Kumar et al., (2012) with the exception of a library PCR enrichment of 11 PCR cycles. RNA sequencing libraries were validated using high sensitivity DNA chips on the Agilent Bioanalyzer and quantified using the Quant-iT dsDNA Assay kit (ThermoFisher Scientific). 50 bp single-end RNA-sequencing was carried out at the UC Davis genomics core facility (Davis, Calif.) on the Illumina HiSeq 2500 platform in high throughput mode. All data has been deposited in the Gene Expression Omnibus (GEO) data repository (accession GSE77723).

### Data Analysis.

Barcode adaptors from the RNA sequence reads were clipped and low quality reads removed (read quality<30) using the Trimmomatic software (Bolger et al., 2014). Quality control of each sample was performed with FastQC reports (available on the world wide web at bioinformatics.babraham.ac.uk/projects/fastqc/). RNA sequence reads passing quality filter were aligned to the B. napus genome (v4.1, Chalhoub et al., 2014) with Tophat2 of the Tuxedo pipeline (Trapnell et al., 2012) allowing no more than two mismatches, in high sensitivity mode, using B. napus reference annotation v5.0 as a guide (Chalhoub et al., 2014), and otherwise used default settings. Identification of unannotated transcripts was performed using cufflinks v2.2.1 and CuffMerge (Trapnell et al., 2012) and transcript sequences were extracted using BedTools. Novel transcripts were identified and are defined in Data S4. Open reading frames (ORFs) were identified using TransDecoder (available on the world wide web at transdecoder.github.io) with alignment against Arabidopsis TAIR10 using ncbi-BLAST (Altschul et al., 1990). The BLASTp function was used when a predicted protein sequence was available, with an e-value cutoff of e-10. For those without a predicted ORF or no hit, blastn was used to identify potential orthologs (e-value: e-10).

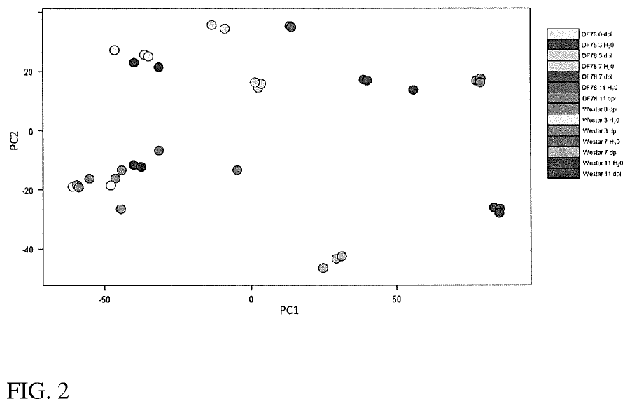

Cuffquant, CuffNorm and Cuffdiff were used to generate normalized counts in FPKM (also known as RPKM in single-ended sequencing (Mortazavi et al., 2008; Trapnell et al., 2012)) and to identify DEGs (pooled dispersion method/standard settings). Genes were considered significantly differentially expressed with a corrected p-value of <0.05 (false discovery rate=0.05). Raw counts were obtained from BAM files using the HTSeq Python Framework with the following command “htseq-count -m union -f bam —stranded=no input.sam bnapusannotation.gff3”. Following, clustering was performed using the averaged raw counts of genes differentially expressed in one or more treatment group. Clustering was performed with the DESeq software package (Anders and Huber, 2010). Principle component analysis was also performed with DESeq using raw counts from each individual sample and validates clustering analysis (FIG. 2).

### Gene Ontology (GO) Term Enrichment.

GO term enrichment was performed per the methods of Orlando et al. 2009. A hypergeometric distribution test was used to identify statistically enriched GO terms overrepresented in lists of DEG sets and assigned a p-value. GO terms were considered statistically enriched at p<0.001. GO attributes were assigned to B. napus genes by transferring GO attributes of their closest putative Arabidopsis homolog (TAIR10; available on the world wide web at arabidopsis.org).

Tissue processing for laser microdissection, RNA isolation, cDNA synthesis and qPCR.

Inoculated cotyledons were collected and processed for LMD per the methods of Belmonte et al. (2013). Briefly, infection sites were cut parallel to the cotyledon petiole-like structure on either side of the lesion between 11:00AM-2:00PM to minimize time of day effect. A minimum of 16 infection sites per biological replicated were collected from the four treatments were fixed in 3:1 (v/v) ethanol:acetic acid and fixed overnight at 4° C. Tissues were then rinsed and dehydrated in a graded ethanol series (75%, 85%, 95%, 100%, 100%) followed by xylene infiltration (3:1, 1:1, 1:3 ethanol:xylene (v/v), 100% xylenes, 100% xylenes) at 4° C. overnight. Tissues were washed with 100% xylene and paraffin chips were added to the xylene infiltrated tissue and kept at 4° C. overnight. Paraffin chips and tissue in xylenes were then allowed to come to room temperature and incubated at 42° C. for 30 minutes followed by 60° C. for 1 hour. Three changes of 100% paraffin were made every hour before embedding.

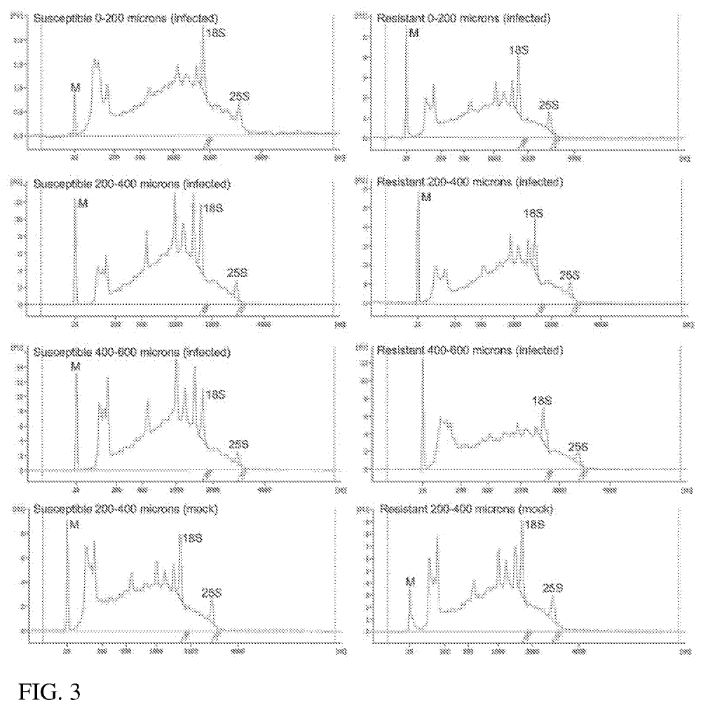

Cotyledon tissues were sectioned using a Leica RM2125RT rotary microtome at 10 μm under RNAse-free conditions and mounted on Leica PEN Membrane slides before being deparaffinized in xylene two times for 30 seconds per wash. Histological sections 0-200, 200-400 and 400-600 μm from the edge of the infection site were collected into 60 μl of lysis buffer (Ambion, Origin). RNA was isolated from sections totaling at least 9000000 μm2 (ranging from 115 to 200 microdissected sections) from at least 7 plant individuals exactly as reported in Belmonte et al. (2013). RNA quality and yield was determined using microcapillary electrophoresis (Agilent 2100 bioanalyzer using an RNA 6000 pico chip). Several examples of RNA traces used to assess RNA quality can be found in FIG. 3. All LMD-collected tissues were of sufficient quality for downstream transcriptome profiling as described in Millar et al. (2015) and Chan et al. (2016).

Isolated RNA was converted to cDNA using the Maxima First Strand cDNA synthesis kit (Thermo Fisher Scientific Inc.). Directed qPCR was carried out using a Bio-Rad CFX Connect™ Real-Time System with SYBR Green Supermix (Bio-Rad, USA) as per manufacturer's instructions in a 10 μl reaction volume. Conditions for the reaction were as follows: 95° C. for 3 min, 39 cycles of 95° C. for 30 s, 53° C. for 30 s, and 72° C. for 30 s. Melt curves (0.5° C. increments in a 55-95° C. range) for each gene were performed to assess the sample for non-specific targets, splice variants, and primer dimers. A list of the primer sequences used in these experiments is found in Table 1. The ΔΔCt method was used to analyze relative transcript abundance, normalizing to the endogenous housekeeping gene Actin and using Westar inoculated with H₂O as a reference sample.

TABLE 1
Primer sequences used for LMD-qPCR and 18s rDNA detection.

| B. napus LMD | | | |
|---|---|---|---|
| Primers | Sequence (5′ --> 3′) | Primer Efficiency | SEQ ID No. |
| qBnaPR1.F | TCTCGTTGACCCAAAGGTTC | 83.70% | 109 |
| qBnaPR1.R | CAGCCTTCGCTCAAAGCTAC | | 110 |
| qBnaPDF1.2.F | GCTGCTTTTGAAGCACCAAC | 84.38% | 111 |
| qBnaPDF1.2.R | GTTGCAAGATCCATGTCGTG | | 112 |
| qBnaCYP79B2.F | TCAACGCGTGTCTCATTCTC | 91.94% | 113 |
| qBnaCYP79B2.R | TACCGGGAAAAGAGGTTGTG | | 114 |
| qBnaC04g27200D.F | TCGTCTAGGCCAAGTTCGTC | 75.19% | 115 |
| qBnaC04g27200D.R | AAAGAAGAAGCGGCAACAAG | | 116 |
| qBnaLIK1.F | TTGGCACTTCCCCACTTAAC | 85.19% | 117 |
| qBnaLIK1.R | GCGTATCTTGGACCGATCAC | | 118 |
| qBnaAPSK2.F | GTTGGGAGCCTTAGGAAACC | 94.91% | 119 |
| qBnaAPSK2.R | ACCGTCCATCATCTGCTCTC | | 120 |
| qBnaA03g43720D.F | TAGGCTGTGACGGGACTACC | 91.92% | 121 |
| qBnaA03g43720D.R | TCCGGCTTCATAGAATGTCC | | 122 |
| BnWRKY25.F | TTCACCGACCTCCTTGCTTC | 97.52% | 123 |
| BnWRKY25.R | GAAGCTGCTGCGAGAAGATTGCG | | 124 |
| BnRBOHF.F | CTTGGCATTGGTGCAACTCC | 80.02% | 125 |
| BnRBOHF.R | TCCGAGARCGAATCCGCTTG | | 126 |
| qLmActin.F | ATCTCTTGGTTCTGGCATCG | 80.52% | 127 |
| qLmActin.R | GCAATGTGCGTTCAAAGATT | | 128 |

The ΔΔCt method was used to analyze relative mRNA abundance (Rieu and Powers, 2009). The results are based on three repeats in three independent experiments. The ΔΔCts of the replicates for each sample and distance, containing tissue from at least 7 individuals. Actin (GenBank accession number: AF111812.1) was used as the internal control to normalize the expression of the target gene. Levels of gene expression were normalized relative to that in Westar (0-200 μm) control.

One-way ANOVA with Ducan's multiple range test (p<0.05) was performed on each gene over the three distances to test for significant fold changes between treatments (p<0.05).

### Arabidopsis Susceptibility Screening.

We screened 49 loss-of-function Col-0 background Arabidopsis mutants for susceptibility to L. maculans (Table 2). PCR was performed to confirm homozygous insertion of the mutants. Col-0 plants were used as a resistant control line and mock water-inoculated controls were performed for all lines. Plant growth and fungal inoculation procedures were similar as described in B. napus plant growth and fungal inoculation, with some modifications. Seeds were plated in MS medium in sterile conditions, then cold-treated for three days at 4° C., incubated in controlled environment for 14 days, and transplanted into growth tray with growth mix. Inoculation of two similarly-sized young leaves per plant was performed at the 4-6 leaf stage, and after inoculation a transparent plastic cover was placed over the plants to maintain high humidity. At least 30 plants from each treatment group were evaluated for blackleg resistance at 18-24 dpi and scored for disease severity.

TABLE 2
Results of Arabidopsis mutant susceptibility screening for
blackleg disease. A total of 49 loss-of-function Col-0
background Arabidopsis mutants were screened for blackleg
disease susceptibility. Blackleg resistance evaluation:
R, resistant, infected plants showed small lesions with clear
black borders; +, some visual evidence for marginal breakdown of
non-host resistance where fungal load increase not significant
in qPCR assays; ++ lesions spread into host tissues and
infected hosts have significantly (p < 0.05) higher fungal loads;
+++ reproductive structures (pycnidia) of fungus are visible
and hosts have significantly higher (p < 0.05) fungal loads.

| | T-DNA | | Insertion | Blackleg |
|---|---|---|---|---|
| Mutant | insertion line | Gene name | site | resistance |
| N/A | Col-0 | N/A | N/A | R |
| at3g11820 | SALK_087016C | PEN1 | Promoter | ++ |
| at3g53490 | SALK_036238 | u/c | Promoter | + |
| at3g14840 | SALK_030855C | LIK1 | Exon | + |
| at4g18250 | SALK_043853C | u/c | Intron | ++ |
| at4g18250 | SALK_072295C | u/c | Promoter | ++ |
| at1g73260 | SALK_131716C | KTI1 | Promoter | +++ |
| at4g39940 | SALK_025296C | APK2 | Exon | ++ |
| at4g39940 | SALK_060023C | APK2 | Promoter | + |
| at3g14840 | SALK_056862 | LIK1 | Promoter | R |
| at1g02930 | SALK_026398C | GSTF6 | Intron | R |
| at4g21120 | SALK_087921C | AAT1 | Exon | R |
| at4g21120 | SALK_059873C | AAT1 | Intron | R |
| at1g33950 | SALK_000761C | u/c | Intron | R |
| at1g02930 | SALK_065940C | GSTF6 | Exon | R |
| at4g17500 | SALK_036267 | ERF-1 | Promoter | R |
| at4g04540 | SALK_098187C | CRK39 | Exon | R |
| at3g60420 | SALK_057524C | u/c | promoter | R |
| at3g60420 | SALK_059036C | u/c | promoter | R |
| at3g61640 | SALK_092212C | AGP20 | promoter | R |
| at3g05360 | SALK_008911C | RLP30 | Exon | R |
| at3g05360 | SALK_145342C | RLP30 | Exon | R |
| at4g23290 | SALK_022512C | CRK21 | Exon | R |
| at4g23290 | SALK_035263C | CRK21 | Exon | R |
| at4g22880 | SALK_120680C | LDOX | Promoter | R |
| at4g22880 | SALK_073183 | LDOX | Exon | R |
| at4g04540 | SALK_036225C | CRK39 | Exon | R |
| at4g11850 | SALK_089968 | LPLDGAMMA1 | Promoter | R |
| at3g53490 | SALK_645697C | u/c | 5′ UTR | R |
| at5g14930 | SALK_022911C | SAG101 | Exon | R |
| at5g01750 | SALK_089519C | u/c | Promoter | R |
| at5g01750 | CS372146 | u/c | Promoter | R |
| at4g23190 | SALK_054888 | CRK11 | Exon | R |
| at4g23190 | SALK_054880 | CRK11 | Exon | R |
| at5g53110 | SALK_136256 | u/c | Exon | R |
| at5g53110 | SALK_004123 | u/c | Intron | R |
| at3g25882 | SALK_148447C | NIMI-2 | Exon | R |
| at3g25882 | SALK_06674C | NIMI-2 | Promoter | R |
| at2g30860 | SALK_148672C | GSTF9 | Promoter | R |
| at2g30860 | SALK_001519C | GSTF9 | Exon | R |
| at1g66880 | SALK_034755 | u/c | Exon | R |
| at1g66880 | SALK_137021 | u/c | Exon | R |
| at5g17220 | SALK_105779C | u/c | Intron | R |
| at5g17220 | SALK_113805C | u/c | Promoter | R |
| at5g41020 | SALK_108569C | u/c | Promoter | R |
| at1g74650 | CS2104374 | MYB31 | Promoter | R |
| at4g39950 | SALK_113348C | CYP79B2 | Exon | R |
| at4g31500 | SALK_102615 | CYP83B1 | Promoter | R |
| at1g26420 | SALK_079007 | u/c | Promoter | R |
| at2g46650 | SALK_027748C | CYTB5-C | Exon | R |
| at1g11330 | SALK_076543C | u/c | Promoter | R |

Leaf tissue was collected in a 96-well plate from five biological replicates of Arabidopsis wild-type plants and mutants that displayed susceptibility at 20 dpi. DNA extraction buffer (1M KCl, 100 mM Tris-HCl pH 7.5, 10 mM EDTA pH 8) and glass beads were added to each well and tissue homogenized on the GenoGrinder 2000. DNA was precipitated in isopropanol, washed with 70% ethanol, and suspended in Tris-HCl pH 7.5. To properly normalize input for qPCR DNA was quantified with the Nanodrop 2000c and Quant-iT picogreen high sensitivity dsDNA assay (Thermo Fisher Scientific Inc.) on the fluorescent Nanodrop 3300. To measure 18s rDNA levels in foliar tissue, qPCR was performed with SYBR SSO Fast Evagreen Supermix (Bio-Rad, USA) in a 10 μl reaction volume. For each reaction, 100 pg of extracted DNA was used. Conditions for the reaction were as follows: 98° C. for 3 min, 40 cycles of 98° C. for 5 seconds, 60° C. for 10 seconds. Melt curves (0.5° C. increments in a 55-95° C. range) for each gene were performed to assess for non-specific targets and primer dimers.

### Results

The LepR1-AvrLepR1 gene Interaction is Responsible for Resistance in DF78 Cotyledons.

To better understand the host-pathogen relationship between B. napus and L. maculans, we performed cotyledon inoculation assays based on the gene-for-gene model developed by Flor (1971) frequently applied in the characterization of R-genes (Rouxel et al., 2003; Marcroft et al., 2012). A total of 34 characterized L. maculans isolates were tested against 104 B. napus varieties/lines (Zhang et al., 2016). We selected resistant line DF78 (LepR1) for further analysis because of its strong defense response against L. maculans (AvrLepR1) and our interest in the poorly characterized R-gene LepR1. Our results show DF78 is resistant to all isolates carrying AvrLepR1 or AvrLm3. As the L. maculans isolate D3 used for this study does not carry AvrLm3 (Table 3; Zhang et al., 2016), the response of DF78 cotyledons to the D3 L. maculans isolate must be the result of a LepR1—AvrLepR1 gene interaction. To confirm, B. napus variety Q2 (Rlm3; Van de Wouw et al., 2010) and B. napus line 1065 (LepR1; Zhang et al., 2016) were used as controls. When Westar was challenged with all 34 isolates, no resistance was observed, confirming previous reports that Westar is universally susceptible to L. maculans (Table 3).

TABLE 3
Characterization of R-genes carried in resistant line DF78 and susceptible
cv. Westar. A total of 34 characterized L. maculans isolates were tested
against cv. DF78 and cv. Westar and interaction phenotype was recorded
as resistant [R] or susceptible [S]. The genotype of Avr genes enclosed in
( ) are not determined.

| | | Inter- | Inter- |
|---|---|---|---|
| | | action | action |
| | | with | with |
| Isolates | Avirulence genotype | DF78 | Westar |
| D1 | AvrLm2, 5, 6, 9, (10), S, AvrLepR1, 2 | R | S |
| D2 | AvrLm5, 6, 8, (10), 11, S, AvrLepR1 | R | S |
| D3 | AvrLm5, (10), 11, AvrLepR1 | R | S |
| D4 | AvrLm4, 5, 6, 7, 8, (10), 11, AvrLepR1, 2 | R | S |
| D5 | AvrLm1, 2, 4, 7, (10), 11, S, AvrLepR1, 2 | R | S |
| D6 | AvrLm1, 5, 6, 8, (10), 11, S | R | S |
| D7 | AvrLm1, 3, 5, 6, 8, (10), 11, (S), AvrLepR1 | R | S |
| D8 | AvrLm5, 7, (8, 10), 11, AvrLepR1 | R | S |
| D9 | AvrLm5, 6, 7, (8, 10), 11, AvrLepR1 | R | S |
| D10 | AvrLm5, 6, 8, 9, (10), 11, S | R | S |
| D13 | AvrLm4, 6, 7, (8, 10), 11 | S | S |
| D14 | AvrLm1, 7, (5, 8, 10), 11, S, AvrLepR1 | R | S |
| S7 | AvrLm1, 5, 6, 7, (8), 11, AvrLepR1 | R | S |
| ICBN14 | AvrLm5, 6, 10, AvrLepR1 | R | S |
| PHW1223 | AvrLm5, 6, 8, 9, 11 | R | S |
| R2 | AvrLm5, 7, 10, (8), AvrLepR1 | R | S |
| AD746 | AvrLm3, 6, (8), AvrLepR1 | R | S |
| JN2 | AvrLm5, 6, 7, 8, 11, AvrLepR1 | R | S |
| JN3 | AvrLm1, 4, 5, 6, 7, 8, 11 | R | S |
| J3 | AvrLm2, 3, 5, 6, (8, 10), 11, S | R | S |
| J20 | AvrLm2, 3, 6, (8, 10), 11, S, AvrLepR1 | R | S |
| Q12 | AvrLm2, 4, 5, 7, (8, 10), 11, AvrLepR1 | R | S |
| L-MD7-14 | AvrLm4, 5, 6, 7, (8, 10), 11 | S | S |
| L-PC4-1 | AvrLm2, 4, (8, 10), 11 | S | S |
| L-MP1-8 | AvrLm2, 4, 5, 6, 7, (8, 10), 11 | S | S |
| L-Sb1 | AvrLm2, 3, 5, 6, 7, (8, 10),S, 11 | R | S |
| L-MP1-6 | AvrLm4, 5, 6, 7, (8, 10), 11 | S | S |
| L-Sb7-6 | AvrLm4, 5, 6, 7, (8, 10), 11, LepR1 | R | S |
| L-Br17-1 | AvrLm5, 6, 7, (4, 8, 10), 11, LepR1 | R | S |
| L-Mo5-1 | AvrLm2, 4, 5, 6, 7, (8, 10), 11, LepR2 | S | S |
| L-Br1-16 | AvrLm1, 4, 5, 6, 7, (8, 10, S), 11 | S | S |
| RL25 | AvrLm5, 6, 7, (8, 10), 11, S | S | S |
| DS103 | AvrLm5, 9, (8, 10), 11 | S | S |
| CV8-7 | AvrLm2, 4, 5, 6, 7, (5, 8, 10), 11, S | S | S |

Phenotypic and Cellular Characterization of B. napus Cotyledons in Response to L. maculans Infection.

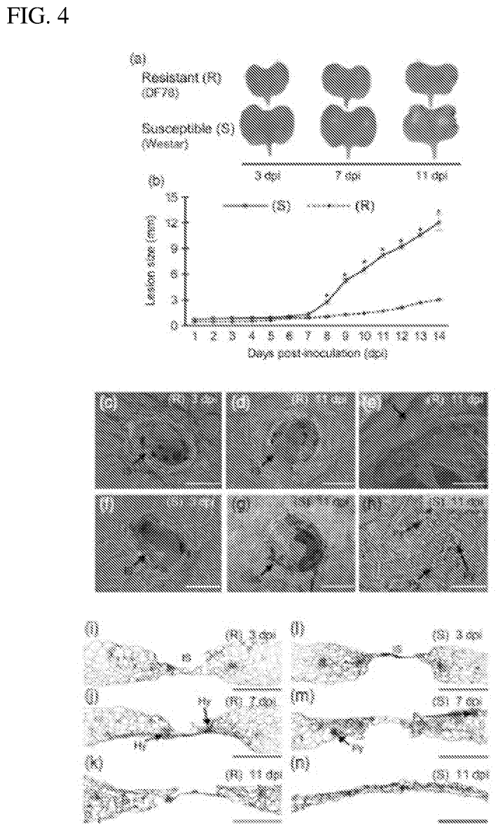

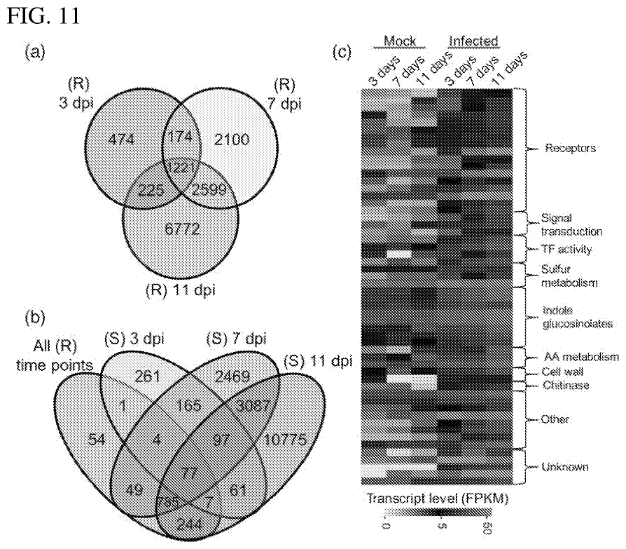

Next, the phenotypic characteristics of resistant (DF78; LepR1) and susceptible (Westar) B. napus hosts infected with L. maculans (FIG. 4a) were examined. Lesions spread rapidly in susceptible cotyledons at 7 days post-inoculation (dpi), while in resistant hosts lesion size only slightly increased towards the end of the 14-day infection period (FIG. 4b). Scanning electron and light microscopy of resistant cotyledons showed minimal cellular breakdown adjacent to the infection site at 3 and 7 dpi (FIG. 4c-d, i-j), as is characteristic of ETD responses, despite the presence of fungal hyphae within the infection site (FIG. 4e) and by 11 dpi, resistant hosts show marginal cellular degradation (FIG. 4k). In susceptible hosts, cells adjacent to the infection site were intact at 3 dpi (FIG. 4f) and widespread cell death by 7 (FIGS. 4m) and 11 dpi (FIG. 4g, n) with fungal fruiting bodies clearly visible (FIG. 4h, m).

Global Comparison of Gene Activity in the B. napus-L. maculans Pathosystem.

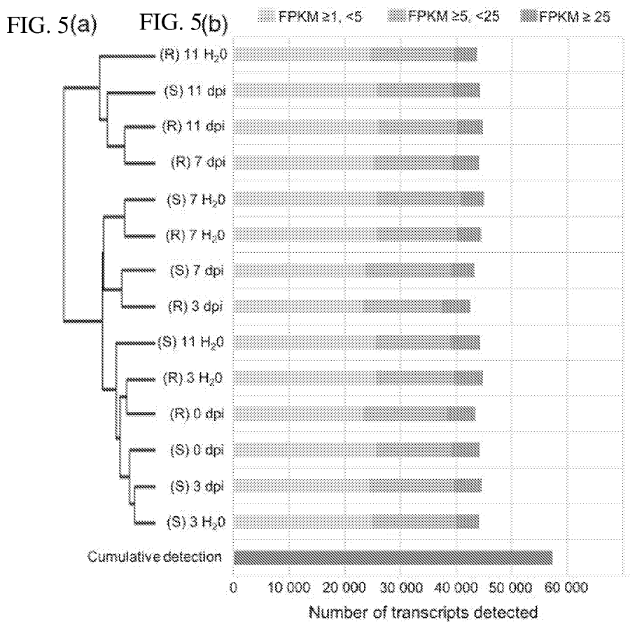

To identify genes responsible for B. napus resistance to L. maculans, we profiled the transcriptomes of resistant and susceptible cotyledons using next generation RNA sequencing across a two-week infection period. First, hierarchical clustering analysis revealed relationships between genotypes and in response to L. maculans infection (FIG. 5a). Treatments generally grouped according to genotype at 0-3 dpi, apart from infected resistant cotyledons at 3 dpi that cluster with susceptible plants 7 dpi suggesting an accelerated defense response. Towards the latter stages of the infection process, treatments form a clade based largely on exposure to L. maculans, highlighting global shifts in gene expression in both genotypes following pathogen attack. Mock-inoculated resistant plants at 11 dpi were also placed within this clade, which may be related to its developmental profile and shared activation of senescence-associated genes.

FIG. 5b summarizes transcript populations in both genotypes and across treatments. Transcript abundance was measured as Fragments Per Kilobase of gene per Million mapped reads (FPKM) where a gene was scored as ‘expressed’ when FPKM≥1 (Mortazavi et al., 2008; Trapnell et al., 2012; Bhardwaj et al., 2015). Regardless of genotype or treatment, the number of active genes was similar, with an average of 41,110 expressed genes (41% of the B. napus gene models). Transcript abundance was scored as low (FPKM≥1, <5), moderate (FPKM≥5, <25), or high (FPKM≥25), with the majority of transcripts detected at low (53%) or moderate (36%) levels. Cumulatively, 57,654 transcripts were detected across all 12 treatments with an FPKM≥1.

Thousands of Genes are Activated in B. napus in Response to L. maculans.

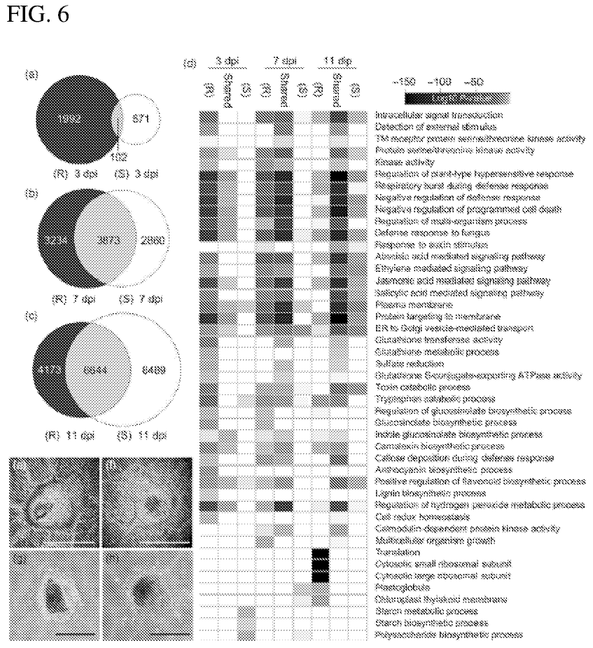

To identify genes contributing to plant resistance, differential gene expression analysis was performed at all stages of the 11-day infection process in both resistant and susceptible hosts and data compared to their respective mock, water-inoculated controls. At 3, 7, and 11 dpi, we detected a total of 1992, 3234, and 4173 upregulated differentially expressed genes (DEGs, p<0.05) in resistant- and 571, 3873, and 8489 upregulated DEGs in susceptible hosts, respectively (FIG. 6a-c). The number of DEGs shared between resistant and susceptible host cotyledons also increased over time and likely due to the total number of DEGs between treatments.

Host resistance is associated with pathogen recognition, cell signaling, and vesicular trafficking in resistant plants.

To identify the biological processes, molecular functions, and cellular components contributing to host resistance against L. maculans, we performed Gene Ontology (GO) term enrichment on all upregulated DEG sets (FIG. 6d). DEGs identified in resistant cotyledons at 3 dpi are enriched with kinase activity (P=1.05E-13), signal transduction (P=1.5E-04), and plasma membrane (P=2.85E-30), and code for wall-associated kinases (WAKs), RLKs, RLPs, LRR-NBS receptors, and transducers of signaling such as MAPKs and MAPK kinases (MKK). Specifically, we identified two putative homologs of RLP30 (BnaA06g12200D, BnaA06g12220D), receptor complex regulator SUPPRESSOR OF BIR1 1 (SOBIR1, BnaA03g14760D, BnaCnng39490D), and homologs of signal transducer MKK9 (BnaA02g35860D, BnaC02g22230D) that were upregulated specifically in resistant cotyledons at 3 dpi (Table 4).

TABLE 4
Accumulation of transcripts during L. maculans infection in resistant (R) and
susceptible (S) B. napus cotyledons. Significant (P < 0.05) decrease or increase in
transcript abundance as compared to mock controls are in bold.

| | | Fold Change vs. Mock Control | | | | | |
|---|---|---|---|---|---|---|---|
| | | R | R | R | S | S | S |
| B. napus locus | Putative annotation | 3 dpi | 7 dpi | 11 dpi | 3 dpi | 7 dpi | 11 dpi |
| BnaA03g46200D | PUTATIVE NBS-LRR | 2.16 | 6.03 | 3.02 | 0.85 | 10.46 | 26.07 |
| | RECEPTOR | | | | | | |
| BnaC04g12970D | PUTATIVE NBS-LRR | 2.12 | 3.55 | 1.40 | 0.54 | 2.40 | 12.61 |
| | RECEPTOR | | | | | | |
| BnaA03g14760D | SUPRESSOR OF BIR1 1 | 2.10 | 5.12 | 2.13 | 1.43 | 2.80 | 21.13 |
| BnaCnng39490D | SUPRESSOR OF BIR1 1 | 2.99 | 3.86 | 3.19 | 1.36 | 3.74 | 7.21 |
| BnaC04g43230D | RECEPTOR-LIKE PROTEIN 30 | 4.60 | 12.75 | 3.12 | 0.70 | 4.92 | 37.27 |
| BnaA06g12200D | RECEPTOR-LIKE PROTEIN 30 | 2.97 | 5.90 | 1.28 | 1.41 | 1.54 | 12.65 |
| BnaA04g06980D | CRK10 | 5.12 | 3.29 | 14.21 | 0.42 | 0.82 | 17.56 |
| BnaA02g21140D | CRK39 | 5.20 | 41.07 | 10.07 | 1.12 | 27.26 | 205.9 |
| BnaA02g35860D | MAP KINASE KINASE 9 | 2.00 | 2.86 | 2.30 | 0.64 | 1.72 | 12.94 |
| BnaC02g22230D | MAP KINASE KINASE 9 | 5.39 | 5.27 | 2.41 | 0.40 | 4.90 | 25.43 |
| BnaA08g17130D | SEC23/24 TRANSPORT GENE | 0.99 | 2.40 | 0.80 | 1.41 | 0.82 | 2.20 |
| BnaC03g73490D | SYNTAXIN OF PLANTS 121 | 1.03 | 1.71 | 1.86 | 1.85 | 1.05 | 7.90 |
| BnaA07g30760D | KUNITZ TRYPSIN INHIBITOR 1 | 2.69 | 3.51 | 9.31 | 0.59 | 0.03 | 0.14 |
| BnaC09g20030D | BAX INHIBITOR 1 | 1.82 | 3.08 | 4.53 | 1.39 | 11.54 | 38.20 |
| BnaC03g58590D | NECROTIC SPOTTED LESIONS | 1.70 | 1.98 | 1.70 | 1.31 | 1.70 | 19.29 |
| | 1 | | | | | | |
| BnaC03g22580D | NUDIX HYDROXYLASE H7 | 5.53 | 17.96 | 11.44 | 1.54 | 39.82 | 27.34 |
| BnaC01g41070D | BOTRYTIS SUSCEPTIBLE 1 | 1.66 | 1.08 | 1.18 | 0.64 | 0.69 | 6.87 |
| | INTERACTOR | | | | | | |
| BnaC06g13910D | DEFENDER AGAINST DEATH 1 | 1.81 | 1.83 | 1.74 | 1.28 | 0.55 | 45.89 |
| BnaA07g15670D | DEVELOPMENT AND CELL | 2.73 | 1.30 | 2.20 | 0.99 | 1.00 | 28.99 |
| | DEATH 1 | | | | | | |
| BnaC09g50680D | SULFITE REDUCTASE 1 | 1.77 | 2.62 | 0.97 | 0.69 | 1.29 | 1.05 |
| BnaA03g38670D | APK1 | 2.65 | 5.89 | 6.69 | 1.27 | 0.81 | 3.16 |
| BnaA01g34620D | APK1 | 3.37 | 4.87 | 25.01 | 0.59 | 0.83 | 2.15 |
| BnaA09g20370D | APS REDUCTASE 1 | 2.85 | 2.40 | 1.79 | 1.14 | 5.60 | 6.53 |
| BnaC09g22760D | APS REDUCTASE 1 | 2.27 | 1.19 | 1.32 | 1.24 | 12.51 | 5.02 |
| BnaA06g28850D | GLUTATHIONE SYNTHETASE 2 | 1.55 | 2.01 | 1.94 | 0.99 | 1.64 | 1.87 |
| BnaC07g27830D | GLUTATHIONE SYNTHETASE 2 | 1.87 | 1.81 | 1.85 | 1.03 | 0.84 | 1.78 |
| BnaC09g40740D | GLUTATHIONE S-TRANSFERASE PHI 12 | 10.46 | 0.44 | 0.25 | 0.20 | 10.13 | 0.09 |
| BnaA07g24870D | LIPOXYGENASE 2 | 1.00 | 19.09 | 13.06 | 0.00 | 0.00 | 0.05 |
| BnaA07g24880D | LIPOXYGENASE 2 | 1.89 | 18.74 | 23.19 | 0.21 | 0.00 | 0.04 |
| BnaA04g17560D | CINNAMATE-4-HYDROXYLASE | 27.64 | 15.61 | 1.48 | 1.50 | 1.61 | 90.95 |
| BnaC04g41120D | CINNAMATE-4-HYDROXYLASE | 18.56 | 3.00 | 1.61 | 0.77 | 1.53 | 40.45 |
| BnaA07g32800D | CINNAMOYL-COA REDUCTASE | 21.61 | 45.49 | 32.21 | 1.29 | 116.69 | 206.3 |
| BnaA08g16100D | CYP79B2 | 1.68 | 13.03 | 9.54 | 1.38 | 1.70 | 1.99 |
| BnaA08g04520D | CYP83B1 | 1.78 | 2.07 | 3.70 | 0.86 | 0.64 | 0.78 |
| BnaC04g01210D | WRKY46 | 2.43 | 3.07 | 2.18 | 1.07 | 11.31 | 11.3 |
| BnaA04g23480D | WRKY54 | 2.49 | 6.85 | 3.24 | 1.17 | 4.65 | 8.72 |
| BnaA09g35840D | WRKY70 | 3.32 | 12.87 | 23.49 | 1.47 | 27.31 | 24.71 |
| BnaC06g05910D | ANAC019 | 3.09 | 2.76 | 1.95 | 0.29 | 0.20 | 191.8 |
| BnaA07g28000D | ANAC019 | 4.11 | 5.69 | 2.33 | 0.16 | 1.36 | 1369.3 |
| BnaC08g18090D | MYB51 | 1.55 | 6.58 | 5.16 | 1.03 | 8.40 | 13.42 |

### SA and JA Signaling are Strongly Affected by the LepR1-AvrLepR1 Gene Interaction.

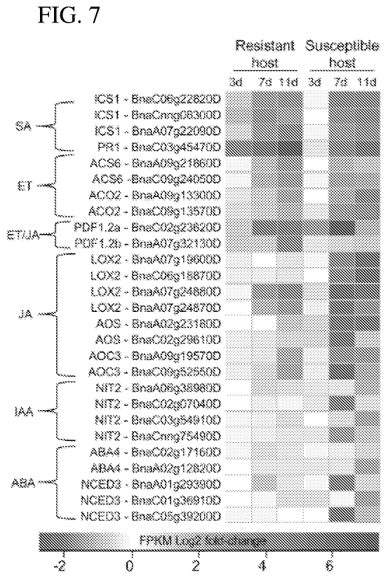

RNA sequencing and GO term enrichment identified DEGs in resistant cotyledons at 3 dpi associated with SA-mediated signaling pathway (P=6.70E-18), ET-mediated signaling pathway (P=6.57E-12), and JA-mediated signaling pathway (P=2.48E-65; FIG. 6d). To further characterize the temporal regulation of hormone production and signaling in response to L. maculans, we examined transcript levels of hormone biosynthetic genes and markers for SA, ET, JA, ABA, and auxin across the infection process in both genotypes (FIG. 7).

Transcript levels of the SA biosynthetic gene homologs, ISOCHORISMATE SYNTHASE 1, in addition to the SA marker PATHOGENESIS-RELATED GENE 1 (PRO increased an average of 5.01-fold against the mock at 3 dpi in resistant plants, as compared to an increase of 1.26-fold in their susceptible counterparts. Data show increased abundance of transcripts related to ET/JA biosynthesis and signaling by 3 dpi in resistant cotyledons, including ACC OXIDASE 2 (BnaA09g13300D, BnaC09g13570D) and ET-JA marker PDF1.2 (BnaA07g32130D, BnaC02g23620D), that continued to accumulate across the infection process. Remarkably, in susceptible hosts, expression levels of several JA-biosynthetic genes decreased. For example, the expression of LIPDXEGENASE 2 (LOX2; BnaA07g24870D, BnaA07g24880D), ALLENE OXIDE SYNTHASE (AOS; BnaC02g29610D), and ALLENE OXIDE CYCLASE 3 (AOC3; BnaC09g52550D) decreased an average of 4.01-fold compared to mock controls at 7 and 11 dpi (FIG. 7). Finally, expression of auxin (NITRILASE 2, BnaA06g38980D, BnaC02g07040D, BnaC03g54910D, BnaCnng75490D) and ABA (NINE-CIS-EPDXYCAROTENOID DIOXYGENASE 3, BnaA01g29390D, BnaC01g36910D, BnaC05g39200D) markers increased in susceptible cotyledons at 11 dpi, and may be the result of widespread cell death late in the infection process (FIG. 4n).

Regulation of Cell Death is Associated With ETD Against L. maculans.

We identified DEGs associated with negative regulation of programmed cell death (P=4.76E-76) upregulated specifically in resistant hosts at 3 dpi (Table 4), including putative homologs of BAX INHIBITOR 1 (BnaC09g20030D), BOTRYTIS SUSCEPTIBLE 1 INTERACTOR (BnaC01g41070D), DEVELOPMENT AND CELL DEATH 1 (BnaA07g15670D), NUDIX HYDROXYLASE HOMOLOG 7 (BnaC03g22580D), METACASPASE 2 (BnaA01g14460D), and NECROTIC SPOTTED LESIONS 1 (BnaC03g58590D). Activation of cell death regulators early during ETD may limit lesion spread following the biotrophic-necrotrophic transition of L. maculans.

### Rapid Activation of Genes Associated With Sulfur Metabolism.

DEGs associated with sulfate reduction (P=1.51E-07), sulfate assimilation (P=1.14E-11), and glutathione metabolic process (P=8.64E-08) were identified specifically in resistant cotyledons at 3 dpi (FIG. 6d), including sulfur assimilators APS REDUCTASE (APR1, BnaA09g20370D, BnaC09g22760D), APR2 (BnaC04g19270D), APR3 (BnaC01g13420D, BnaC07g37060D), and SULFITE REDUCTASE (BnaC09g50680D), as well as sulfate activators ADENOSINE 5′-PHOSPHOSULFATE KINASE 1 (APK1, BnaA03g38670D) and APK2 (BnaA01g34620D, BnaC01g00790D, BnaC07g51290D). Additionally, homologs of GLUTATHIONE SYNTHETASE 2 (BnaA06g28850D, BnaC07g27830D) were upregulated specifically in resistant hosts at 3 dpi (Table 4). In addition to its role as a redox regulator, glutathione is a key intermediary in sulfur metabolism and the largest reservoir of non-protein reduced sulfur in the cell. It also directly serves a role in toxin neutralization through the activity of glutathione-S-transferases (GST). DEGs enriched for glutathione s-transferase (GST) activity (P=2.77E-21) were also identified in resistant hosts at 3 dpi, including GST PHI 2 (GSTF2, BnaA03g26140D), GSTF6 (BnaC05g01540D), GSTF12 (BnaC09g40740D), EARLY RESPONSE TO DEHYDRATION 9 (ERD9, BnaA06g06160D), ERD13 (BnaA03g14150D), and 26 other GSTs.

Coordinated Lignin Deposition is Observed in Resistant Cotyledons Following Infection With L. maculans.

Genes coding for the formation of monolignols, CINNAMATE-4-HYDROXYLASE (BnaA04g17560D, BnaC04g41120D), CINNAMOYL-ALCOHOL DEHEHYDROGENASE 8/ELICITOR-ACTIVATED GENE 3 (BnaC03g61120D), and CINNAMOYL-COA REDUCTASE (BnaA07g32800D), had a combined average 17.6-fold increase in expression following L. maculans infection in resistant hosts at 3 dpi with no appreciable increase in the susceptible genotype (Table 4). Sequencing data are supported by histochemical analyses of lignin deposition at the inoculation sites of both genotypes (FIG. 6e,f; FIG. 8). Resistant hosts showed prominent and coordinated deposition of lignin proximal to the site of pathogen infection and surrounding vasculature. In susceptible hosts, lignin deposition appeared uncoordinated and diffuse.

### Activation of IGS Biosynthetic Genes and Callose Deposition.

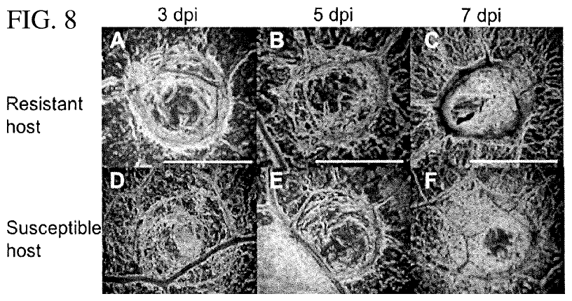

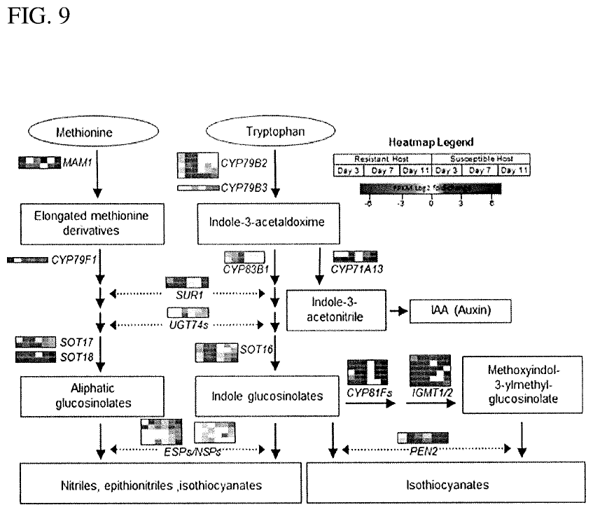

We identified DEGs specific to resistant cotyledons at 3 dpi that are associated with IGS biosynthetic process (P=5.38E-05). In resistant hosts, every gene of the IGS biosynthetic pathway was upregulated following L. maculans infection, whereas in the susceptible genotype several genes required for IGS production, such as CYP79B2 and CYP83B1 (Table 4), were downregulated during infection (FIG. 9). DEGs associated with callose deposition during the defense response (P=1.98E-05) were also identified in resistant cotyledons at 3 dpi, and largely overlapped with the IGS biosynthetic genes and regulators described above. To visualize callose deposition, we stained infected and non-infected cotyledons with aniline blue. Callose accumulated directly adjacent to infection site of resistant cotyledons (FIG. 6g), and was comparatively thin and discontinuous in susceptible hosts (FIG. 6h).

### NAC and WRKY Transcription Factors Are Associated With the Accelerated Defense Response in Resistant Hosts.

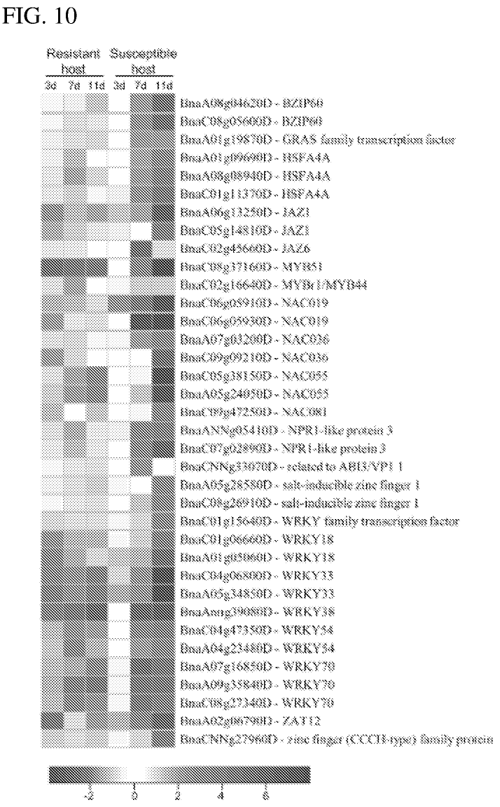

To identify transcription factors (TFs) associated with the accelerated defense response of resistant hosts, we extracted differentially expressed TF-coding genes from the enriched GO terms: regulation of plant-type hypersensitive response (P=1.05E-95), intracellular signal transduction (P=1.54E-23), and defense response to fungus (P=3.03E-93) at 3 dpi. Of the 36 TF-coding transcripts (FIGS. 10), 19.4% and 30.5% coded for members of the NAC and WRKY TF families, respectively. We also identified IGS-promoting MYB51, JA-responsive JAZ TFs, and BZIP60 and HSF-A4A associated with the cellular heat-shock response. Although specifically activated in resistant hosts early at 3 dpi, 94.6% of these transcripts accumulated in susceptible cotyledons to levels exceeding all other treatments by 11 dpi (FIG. 10). These Data suggest the timely expression of TFs may be essential for cellular reprogramming early in the defense response against L. maculans.

### Identification of Genes Specifically Activated by the LepR1-AvrLepR1 Gene Interaction.

To identify genes specifically contributing to resistance in the LepR1-AvrLepR1 interaction, we compared both the susceptible and resistant host transcriptomes across the infection process. We found 1221 upregulated DEGs shared at 3, 7 and 11 dpi in resistant host cotyledons (FIG. 11a). We then compared the 1221 shared DEG in resistant host cotyledons to upregulated DEGs at 3, 7, and 11 dpi in the susceptible host counterpart (FIG. 11b). Of these 1221 DEGs, only 54 were exclusive to resistant host cotyledons. These 54 resistant-specific transcripts included genes involved in signal transduction and gene regulation, such as RLP30 (BnaA06g12220D), CYSTEINE-RICH RECEPTOR-LIKE PROTEIN KINASE 11 (CRK11, BnaA01g12650D), CRK21 (BnaAnng25570D), NON-INDUCIBLE IMMUNITY-INTERACTING GENE 2 (BnaC07g23070D), and ETHYLENE RESPONSIVE ELEMENT BINDING FACTOR 1 (BnaAnng21280D). Further, this list contains two genes associated with sulfur assimilation, SULFATE TRANSPORTER 4.1 (BnaA03g04410D) and APS-KINASE 2 (APK2, BnaC07g51290D), and multiple IGS biosynthetic genes (FIG. 11c). The complete list of 54 resistant-specific genes can be found in Table 5.

TABLE 5
Complete list of 54 genes with significantly (P < 0.5) elevated expression
in response to L. maculans at every time point specifically in resistant line
DF78, and their putative Arabidopsis homolog and annotation. Genes with
no identifiable Arabidopsis homolog from nucleotide or protein BLAST
searches are marked as ‘no hit’ and are of unknown function.

| | Putative | |
|---|---|---|
| | Arabidopsis | |
| B. napus locus | homolog | Putative Annotation |
| BnaC05g38740D | AT3G14840 | LYSM RLK1-interacting kinase 1 |
| BnaA01g12650D | AT4G23190 | cysteine-rich RLK |
| | | (RECEPTOR-like protein kinase) 11 |
| BnaAnng25570D | AT4G23290 | cysteine-rich RLK |
| | | (RECEPTOR-like protein kinase) 21 |
| BnaCnng49020D | AT4G04540 | cysteine-rich RLK |
| | | (RECEPTOR-like protein kinase) 39 |
| BnaA03g25470D | AT4G04540 | cysteine-rich RLK |
| | | (RECEPTOR-like protein kinase) 39 |
| BnaC07g06130D | AT2G17120 | lysm domain GPI-anchored protein |
| | | 2 precursor |
| BnaC03g71330D | AT5G01950 | Leucine-rich repeat protein kinase |
| | | family protein |
| BnaA02g12640D | AT1G66880 | Protein kinase superfamily protein |
| BnaA03g36540D | AT4G11850 | phospholipase D gamma 1 |
| BnaA07g22750D | AT1G73260 | Kunitz Trypsin Inhibitor 1 |
| BnaA07g30760D | AT1G73260 | Kunitz Trypsin Inhibitor 1 |
| BnaA06g12220D | AT3G05360 | receptor like protein 30 |
| BnaA03g43720D | AT4G18250 | Putative receptor kinase |
| BnaC04g27200D | AT3G53490 | Putative receptor kinase |
| BnaA10g07090D | AT1G11330 | S-locus lectin protein kinase family |
| | | protein |
| BnaCnng55880D | AT1G11330 | S-locus lectin protein kinase family |
| | | protein |
| BnaAnng21280D | AT4G17500 | ethylene responsive element |
| | | binding factor 1 |
| BnaA02g25110D | AT5G47220 | ethylene responsive element binding |
| | | factor 2 |
| BnaA09g50010D | AT1G06160 | octadecanoid-responsive Arabidopsis |
| | | AP2/ERF 59 |
| BnaC07g23070D | AT3G25882 | NIM1-interacting 2 |
| BnaA03g04410D | AT5G13550 | sulfate transporter 4.1 |
| BnaC07g51290D | AT4G39940 | APS-kinase 2 |
| BnaCnng04780D | AT1G25220 | anthranilate synthase beta subunit 1 |
| BnaA01g34610D | AT4G39950 | cytochrome P450, family 79, subfamily |
| | | B, polypeptide 2 |
| BnaC01g00800D | AT4G39950 | cytochrome P450, family 79, subfamily |
| | | B, polypeptide 2 |
| BnaA04g12790D | AT2G22330 | cytochrome P450, family 79, subfamily |
| | | B, polypeptide 3 |
| BnaA08g04520D | AT4G31500 | cytochrome P450, family 83, subfamily |
| | | B, polypeptide 1 |
| BnaC08g05690D | AT4G31500 | cytochrome P450, family 83, subfamily |
| | | B, polypeptide 1 |
| BnaA04g27110D | AT2G46650 | cytochrome B5 isoform C |
| BnaC04g50950D | AT2G46650 | cytochrome B5 isoform C |
| BnaC06g21620D | AT1G76790 | Indole Glucosinolate |
| | | O-methyltransferase 5 |
| BnaA03g58530D | AT4G21120 | amino acid transporter 1 |
| BnaA07g23890D | AT1G70260 | Usually multiple acids movie in and out |
| | | transporter 36 |
| BnaA06g31460D | AT3G28480 | Oxoglutarate/iron-dependent oxygenase |
| BnaC03g62400D | AT4G35630 | phosphoserine aminotransferase |
| BnaAnng33720D | AT1G20160 | Response secreted protease |
| BnaC01g41020D | AT4G19810 | Chitinase C |
| BnaAnng42000D | AT4G29700 | Alkaline-phosphatase-like family protein |
| CUFF.2933.3 | AT5G14930 | senescence-associated gene 101 |
| BnaA05g29820D | AT3G14040 | Pectin lyase-like superfamily protein |
| BnaA06g37630D | AT4G04775 | zinc ion binding |
| BnaA04g17910D | AT2G30860 | glutathione S-transferase PHI 9 |
| BnaA09g28900D | AT1G26420 | FAD-binding Berberine family protein |
| BnaA05g07460D | AT2G36970 | UDP-Glycosyltransferase superfamily |
| | | protein |
| BnaC07g47720D | AT4G38540 | FAD/NAD(P)-binding oxidoreductase |
| | | family protein |
| BnaC06g18710D | AT1G21310 | extensin 3 |
| BnaC04g55140D | AT3G60420 | Phosphoglycerate mutase family protein |
| BnaC04g21680D | AT3G61640 | arabinogalactan protein 20 |
| BnaC09g52960D | AT5G53110 | RING/U-box superfamily protein |
| BnaA09g19740D | AT5G01750 | Protein of unknown function (DUF567) |
| BnaC06g28720D | no hit | N/A |
| BnaC02g31360D | no hit | N/A |
| BnaC06g41090D | no hit | N/A |
| BnaA03g08620D | no hit | N/A |

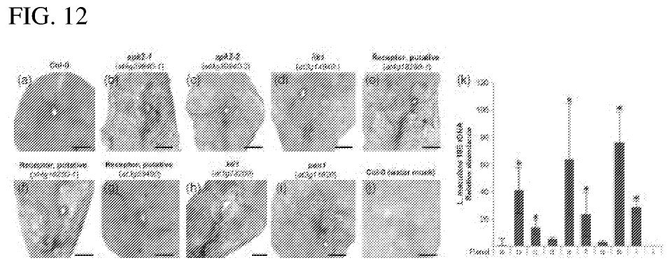

While a non-host to L. maculans, Arabidopsis plants become susceptible to this pathogen if compromised in their ability to detect and/or respond appropriately (Bohman et al., 2004). To functionally characterize the resistant-specific genes identified in our analyses, we challenged 49 corresponding Arabidopsis T-DNA mutants with L. maculans (Table 2). Seven gene disruptions resulted in a breakdown of Arabidopsis non-host resistance by 20 dpi (FIG. 12b-i): apk2-1 and apk2-2, deficient in production of activated sulfur required for biosynthesis of sulfur-containing secondary compounds including IGS and camalexin (Mugford et al., 2009); kunitz trypsin inhibitor 1 (kti1), a negative regulator of phytopathogen induced cell death; receptors at4g18250-1, at4g18250-2, and at3g53490; and receptor partner lysm-interacting kinase 1 (lik1). LIK1, a phosphorylation target of the chitin receptor CERK1, is associated with activation of JA-ET signaling and the repression of SA immune responses (Le et al., 2014). T-DNA mutants of PENTRATION 1 (PEN1), a proven regulator of non-host resistance (Nakao et al., 2011), were used as a positive control and were susceptible to L. maculans. WT Col-0 plants inoculated with L. maculans (FIG. 12a) or water (FIG. 12j) did not show any symptoms associated with infection.

Next, we measured fungal load by qPCR to confirm lesion progression observed in the T-DNA insertion mutants was a result of L. maculans growth and development (FIG. 12k). Fungal load was significantly greater (p<0.05) in all mutants except lik1 (p=0.309) and at3g53490 (p=0.462) suggesting the extent of lesion spread is correlative to fungal load. Other T-DNA alleles of LIK1 and At3g53490 showed no susceptibility to L. maculans (Table 2). This is not surprising, as the effects of T-DNA insertions on gene expression are variable (Wang et al., 2008) and these two mutants already display a weak phenotype. The complete list of screened mutants is found in Table 2.

### Laser Microdissection and Spatial Distribution of Gene Activity Underlying Plant Resistance.

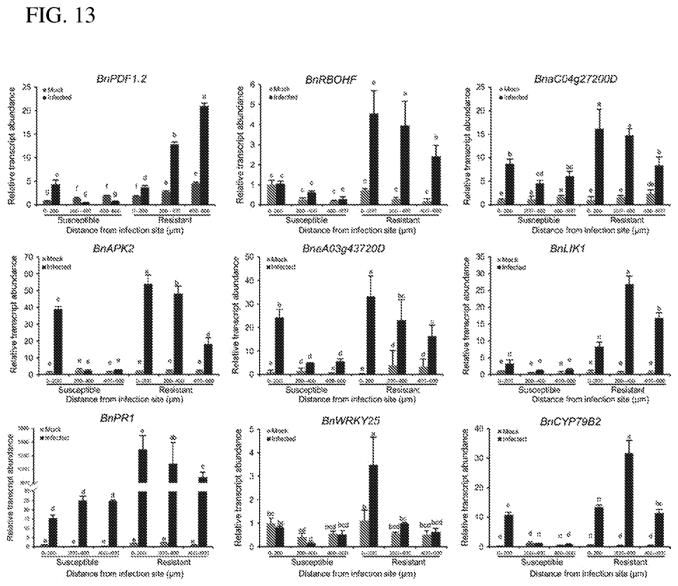

We then hypothesized that the resistant-specific genes identified through our transcriptome and mutant analysis would also be operative directly at the infection site to restrict pathogen spread into host tissues. To test this hypothesis, we used LMD coupled with qPCR to identify how resistant-specific genes and other defense regulators are spatially partitioned within the cotyledon directly at and distal to the infection site (FIG. 13). We focused our attention on cotyledons at 7 dpi; a relevant time point observed between the two genotypes in response to L. maculans (FIG. 4b). All genes (LIK1, PR1, WRKY25, PDF1.2, APK2, RBOHF, CYP79B2, BnaA03g43720D and BnaC04g27200D) were highly expressed in resistant host cotyledons infected with L. maculans compared to the susceptible line or mock controls and further validate our sequencing data.

When resistant host cotyledons were challenged with L. maculans, APK2, RBOHF, WRKY25, BnaA03g43720D, BnaC04g27200D, and SA signaling marker PR1 accumulated at greater levels within tissues 0-200 μm from the infection site. Levels of LIK1 and CYP79B2 were greatest 200-400 μm from the infection site. A marker of JA-ET signaling, PDF1.2, was the only transcript to accumulate highest in tissues taken distally (400-600 μm) from the infection site of resistant hosts. These data provide evidence into the spatial coordination of defense gene activity in tissues directly at the infection site in response to L. maculans attack.

### Discussion

Gene expression in susceptible and resistant cotyledons of B. napus was profiled before, during, and after infection with the hemibiotrophic fungus, L. maculans, to uncover key components of the ETD pathway. Our experiments showed an accelerated defense response in resistant host tissues coinciding with the deposition of lignin and callose that likely prevents L. maculans colonization and reproduction in apoplastic spaces in canola cotyledons. Transcripts associated with resistance accumulated in gradients away from the infection site providing unprecedented spatial resolution into the B. napus-L. maculans pathosystem.

Arabidopsis mutants of two uncharacterized receptors (at4g18250 and at3g53490) were susceptible to L. maculans, suggesting a conserved defensive role in the Brassicaceae. Globally, accelerated defense during ETD is associated with rapid activation of RLPs, RLKs, TIR-NBS receptors, and receptor partner proteins by 3 dpi involved in perception of PAMPs and observed late in the infection process in susceptible cultivars (Haddadi et al., 2016). Of the receptors, 17 were specific to the resistant line and 12 were uncharacterized with no previously described host-pathogen annotation in B. napus, A. thaliana or any other plant pathosystem (Table 5). As ETD pathways are mediated through extracellular RLPs and their associated partner proteins (Stotz et al., 2014), upregulation of these receptors may produce a positive feedback loop amplifying the plant immune response and improving pathogen detection. Furthermore, if ETD and non-host resistance pathways are similar in their architecture, Arabidopsis presents a putative source of effective R-genes with the potential to bolster blackleg resistance in canola.

R-gene efficacy is often independent from the host cell death response (Schiffer et al., 1997; Cawly et al., 2005), suggesting that cell death may not always be responsible for host resistance, but rather a by-product of runaway immune response or cell damage due to infection. Indeed, many necrotrophic or facultatively necrotrophic pathogens will induce host cell death mechanisms to facilitate infection (Lorang et al., 2007; Kabbage et al., 2013), and L. maculans has been shown to produce a necrosis- and ethylene-inducing peptide upon its biotrophic-necrotrophic transition (Haddadi et al., 2016). Phytopathogen-induced cell death repressor KTI1 was induced specifically in resistant hosts. When challenged with L. maculans, lesions spread rapidly in kti Arabidopsis plants and is similar to the phenotype of accelerated cell death 2 plants described by Bohman et al. (2004). Although hemibiotrophic, L. maculans has been defined as primarily necrotrophic (Staal et al., 2008), and can survive within dead or dying plant tissues. Thus, the recognition of L. maculans and activation of cell death regulators early in the infection process likely contribute to delayed onset of cell death observed during ETD. The comparative lack of these regulators early in susceptible hosts may explain its rapid lesion formation following the biotrophic-necrotrophic transition of L. maculans.

JA signaling has been shown to repress hypersensitive-like cell death in Arabidopsis (Rao et al., 2000) and may be an overarching regulator of the genes described above. Susceptible cotyledons show a notable lag in JA response through diminished expression of integral JA biosynthetic genes LOX2, AOS, and AOC, at the time of rapid lesion spread. The expression of NAC TFs early in resistant host cotyledons may directly promote JA production (FIG. 10). For example, NAC019 and NAC055 promote JA-induced transcription of LOX2 (Bu et al., 2008), and anac019anac055 double mutants are susceptible to fungal necrotrophic pathogens (Bu et al., 2008).

Resistance to L. maculans may also involve the production of IGS. Production of IGS is required for resistance against some hemibiotrophic fungi (Hiruma et al., 2013), and in vitro studies have shown S-glycosides from B. napus, predominantly those derived from sinigrin, are toxic to L. maculans (Mithen et al., 1986). Our data show activation of the complete IGS biosynthetic pathway in resistant cotyledons. The production of IGS is linked to sulfur metabolism as all indole-derived phytoalexins in the Brassicas contain sulfur (Pedras et al., 2011). Thus, activation of genes associated with sulfur assimilation during the LepR1-AvrLepR1 interaction supports the production of IGS. Mugford et al. (2009) directly linked sulfur activator APK2 to IGS production in Arabidopsis. Although we have shown that apk2 Arabidopsis plants are susceptible to L. maculans, the mechanism by which susceptibility in conferred is unclear. Other members of the IGS biosynthetic pathway that were challenged, including cyp79b2, cyp79b3, cyp83b1, and cypb5c had no discernable phenotype. The lack of a phenotype in IGS-compromised Arabidopsis plants may be due to complementation by the antifungal indole alkaloid, camalexin, effective against L. maculans (Bohman et al., 2004). As B. napus is unable to produce camalexin, IGS-derived phytoalexins may play a role in defense.

We suspected that key components of the ETD pathway are likely spatially controlled directly at the infection site. Coordination of the ETD pathway, as revealed by LMD and qPCR, increased the spatial resolution of the dataset and show targeted activity of receptors and downstream signal transduction pathways in tissues directly in contact with and those adjacent to L. maculans. While hormone levels are known to flux over time during plant defense, our data show an antagonistic spatial relationship between SA and JA-ET signalling pathways established specifically in resistant host cotyledons as indicated by the distribution of hormone markers PR1 and PDF1.2.

IGS-marker CYP79B2 was highly expressed adjacent to the infection site in an area of combined SA and JA-ET signaling. Consistent with our dataset, Frerigmann and Gigolashvili (2014) found the expression of the main IGS-inducing TF MYB51 was greatest with joint application of SA and JA. Thus, deposition of antifungal IGS-derived phytoalexins most likely does not occur in areas of direct pathogen contact, but rather upstream of invading L. maculans and is potentially guided by hormone gradients formed during defense.

Rapid activation of defense regulators, including TFs, in resistant hosts can contribute to the deposition of lignin, callose, and other anti-fungal metabolites preceding fungal invasion. This is complemented by the ability of resistant plants to direct defense activity to the host-pathogen interface by coordinating gene expression to areas of direct fungal contact or to areas adjacent to the infection site. For example, expression of WRKY25 in resistant host cotyledons is concentrated around 400 microns from the infection site. As a negative regulator of SA-mediated defense responses (Zheng et al., 2007) and a positive regulator of ET biosynthesis (Li et al., 2011), activity of WRKY25 would prevent runaway SA signaling and cell death thus mitigating disease progression and the likelihood of L. maculans colonization.

These data represent a valuable resource that captures gene activity following activation of ETD pathways in the B. napus-L. maculans pathosystem. The identification and characterization of genes responsible for mitigating plant disease demonstrates the utility of our dataset. Further, our data provides a preliminary framework in support of spatial transcriptional gradients responsible for plant resistance. Temporal- and spatial regulation of gene expression both contribute to disease resistance, as expression of all tested genes was tightly controlled at the infection site.

### Citations

Altschul, S. F., Gish, W., Miller, W., Myers, E. W., Lipman and D. J. (1990) Basic local alignment search tool. J. Mol. Biol. 215,403-410.
Anders, S. and Huber, W. (2010) Differential expression analysis for sequence count data. Genome Biol. 11, R106.
Belmonte, M. F., Kirkbride, R. C., Stone, S. L. et al. (2013) Comprehensive developmental profiles of gene activity in regions and subregions of the Arabidopsis seed. Proc. Natl. Acad. Sci. 110, E435-444.
Bhardwaj, A. R., Joshi, G., Kukreja, B. et al. (2015) Global insights into high temperature and drought stress regulated genes by RNA-Seq in economically important oilseed crop Brassica juncea. BMC Plant Biol. 15, 9.
Bigeard, J., Colcombet, J. and Hirt, H. (2015) Signaling mechanisms in pattern-triggered immunity (PTI). Mol. Plant 8, 521-539
Bohman, S., Staal, J., Thomma, B. P. H. J., Wang, M. and Dixelius, C. (2004) Characterisation of an Arabidopsis-Leptosphaeria maculans pathosystem: resistance partially requires camalexin biosynthesis and is independent of salicylic acid, ethylene and jasmonic acid signalling. Plant J. 37, 9-20.
Bolger, A. M., Lohse, M. and Usadel, B. (2014) Trimmomatic: a flexible trimmer for Illumina sequence data. Bioinformatics, 30, 2114-2120.
Bu, Q., Jiang, H., Li, C-B., Zhai, Q., Zhang, J., Wu, X., Sun, J., Xie, Q. and Li, C. (2008) Role of the Arabidopsis thaliana NAC transcription factors ANAC019 and ANAC055 in regulating jasmonic acid-signaled defense responses. Cell Res. 18, 756-767.
Cawly, J., Cole, A. B., Király, L., Qiu, W. and Schoelz, J. E. (2005) The plant gene CCD1 selectively blocks cell death during the hypersensitive response to Cauliflower mosaic virus infection. Mol. Plant-Microbe Interact. 18, 212-219
Chalhoub, B., Denoeud, F., Liu, S., et al. (2014) Early allopolyploid evolution in the post-Neolithic Brassica napus oilseed genome. Science (80), 345, 950-953. Available at: http://www.sciencemag.org/cgi/doi/10.1126/science.1253435.
Chan, A. and Belmonte, M. (2013) Histological and ultrastructural changes in canola (Brassica napus) funicular anatomy during the seed lifecycle. Botany, 91, 671-679.
Chan, A. C., Khan, D., Girard, I. J., Becker, M. G., Millar, J. L., Sytnik, D. and Belmonte, M. F. (2016) Tissue-specific laser microdissection of the Brassica napus funiculus improves gene discovery and spatial identification of biological processes. J. Exp. Bot., 67, erw179.
Clay, N. K., Adio, A. M., Denoux, C., Jander, G. and Ausubel, F. M. (2009) Glucosinolate metabolites required for an Arabidopsis innate immune response. Science, 323, 95-101.
Dangl, J. L., Horvath, D. M. and Staskawicz, B. J. (2013) Pivoting the plant immune system from dissection to deployment. Science, 341, 746-751.
Denancé, N., Sánchez-Vallet, A., Goffner, D. and Molina, A. (2013) Disease resistance or growth: the role of plant hormones in balancing immune responses and fitness costs. Front. Plant. Sci. 4, 1-12.
Ellinger, D., Naumann, M., Falter, C., Zwikowics, C., Jamrow, T., Manisseri, C., Somerville, S. C. and Voigt, C. A. (2013) Elevated early callose deposition results in complete penetration resistance to Powdery Mildew in Arabidopsis. Plant Physiol.161, 1433-1444.
Fitt, B. D. L., Brun, H., Barbetti, M. J. and Rimmer, S. R. (2006) World-wide importance of phoma stem canker (Leptosphaeria maculans and L. biglobosa) on oilseed Rape (Brassica napus). Eur. J. Plant Pathol. 114, 3-15.
Flor, H. H. (1971) Current status of the gene-for-gene concept. Annu. Rev. Phytopathol. 9, 275-296.
Frerigmann, H. and Gigolashvili, T. (2014) MYB34, MYB51, and MYB122 distinctly regulate indolic glucosinolate biosynthesis in Arabidopsis thaliana. Mol. Plant 7, 814-828.
Haddadi, P., Ma, L., Wang, H. and Borhan, M. H. (2016) Genome-wide transcriptomic analyses provide insights into the lifestyle transition and effector repertoire of Leptosphaeria maculans during the colonization of Brassica napus seedlings. Mol. Plant Pathol., 7, 1196-1210.
Hiruma, K., Fukunaga, S., Bednarek, P., Pislewska-Bednarek, M., Watanabe, S., Narusaka, Y., Shirasu, K. and Takano, Y. (2013) Glutathione and tryptophan metabolism are required for Arabidopsis immunity during the hypersensitive response to hemibiotrophs. Proc. Natl. Acad. Sci. 110, 9589-9594.
Jones, J. D. G. and Dangl, J. L. (2006) The plant immune system. Nature, 444, 323-329.
Kabbage, M., Williams, B. and Dickman, M. B. (2013) Cell death control: the interplay of apoptosis and autophagy in the pathogenicity of Sclerotinia sclerotiorum. PLoS Pathog. 9, e1003287.
Kaliff, M., Staal, J., Myrenas, M. and Dixelius, C. (2007) ABA is required for Leptosphaeria maculans resistance via ABI1- and ABI4-dependent signaling. Mol. Plant-Microbe Interact. 20, 335-345.
Kumar, R., Ichihashi, Y., Kimura, S., Chitwood, D. H., Headland, L. R., Peng, J., Maloof, J. N. and Sinha, N. R. (2012) A high-throughput method for illumina rna-seq library preparation. Front. Plant. Sci. 3, 1-10.
Larkan, N. J., Lydiate, D. J., Parkin, I. A. P., Nelson, M. N., Epp, D. J., Cowling, W., Rimmer, S. R. and Borhan, M.H. (2013) The Brassica napus blackleg resistance gene LepR3 encodes a receptor-like protein triggered by the Leptosphaeria maculans effector AVRLM1. New Phytol. 197, 595-605.
Larkan, N. J., Ma, L. and Borhan, M. H. (2015) The Brassica napus receptor-like protein RLM2 is encoded by a second allele of the LepR3/Rlm2 blackleg resistance locus. Plant Biotech. J. 13, 983-992.
Le, M. H., Cao, Y., Zhang, X.C. and Stacey, G. (2014) LIK1, a CERK1-interacting kinase, regulates plant immune responses in Arabidopsis. PLoS ONE, 9, e102245.
Li, S., Fu, Q., Chen, L., Huang, W. and Yu, D. (2011) Arabidopsis thaliana WRKY25, WRKY26, and WRKY33 coordinate induction of plant thermotolerance. Planta, 233.6, 1237-1252.
Lorang, J. M., Sweat, T. A. and Wolpert, T. J. (2007) Plant disease susceptibility conferred by a “resistance” gene. Proc. Natl. Acad. Sci. 104, 14861-14866.
Lowe, R. G. T., Cassin, A., Grandaubert, J., Clark, B. L., Van De Wouw, A. P., Rouxel, T. and Howlett, B. J. (2014) Genomes and transcriptomes of partners in plant-fungal-interactions between canola (Brassica napus) and two Leptosphaeria Species. PLoS ONE, 9, e103098.
Luna, E., Pastor, V., Robert, J., Flors, V., Mauch-Mani, B. and Ton, J. (2011) Callose deposition: a multifaceted plant defense response. Mol. Plant-Microbe Interact. 24, 183-193.
Marcroft, S. J., Van de Wouw, A. P., Salisbury, P. A., Potter, T. D. and Howlett, B. J. (2012) Effect of rotation of canola (Brassica napus) cultivars with different complements of blackleg resistance genes on disease severity. Plant Pathol. 61, 934-944.
Meng, X. and Zhang, S. (2013) MAPK cascades in plant disease resistance signaling. Annu. Rev. Phytopathol. 51, 245-266.
Millar, J. L., Becker, M. G. and Belmonte, M. F. (2015) Laser microdissection of plant tissues. In Plant Microtechniques and Protocols. Springer International Publishing, pp. 337-350.
Mishra, A.K., Sharma, K. and Misra, R. S. (2012) Elicitor recognition, signal transduction and induced resistance in plants. J. Plant Interact., 7, 95-120.
Mithen, R. F., Lewis, B. G. and Fenwick, G. R. (1986) In vitro activity of glucosinolates and their products against Leptosphaeria maculans. Trans. Br. Mycol. Soc. 87, 433-440.
Mortazavi, A., Williams, B. A., Mccue, K., Schaeffer, L. and Wold, B. (2008) Mapping and quantifying mammalian transcriptomes by RNA-Seq. Nat. Methods 5, 1-8.
Mugford, S. G., Yoshimoto, N., Reichelt, M., et al. (2009) Disruption of adenosine-5′-phosphosulfate kinase in Arabidopsis reduces levels of sulfated secondary metabolites. Plant Cell Online, 21, 910-927.
Nakao, M., Nakamura, R., Kita, K., Inukai, R. and Ishikawa, A. (2011) Non-host resistance to penetration and hyphal growth of Magnaporthe oryzae in Arabidopsis. Sci. Rep., 1, 171.
Orlando, D. A., Brady, S. M., Koch, J. D., Dinneny, R. and Benfey, P. N. (2009) Manipulating large-scale arabidopsis microarray expression data: identifying dominant expression patterns and biological process enrichment. In Plant Systems Biology (Belostotsky, D. A., ed). Totowa, N.J.: Humana Press, pp. 57-77.
Pedras, M. S. C., Yaya, E. E. and Glawischnig, E. (2011) The phytoalexins from cultivated and wild crucifers: chemistry and biology. Nat. Prod. Rep., 28, 1381-1405.
Rao, M. V, Lee, H., Creelman, R. a, Mullet, J. E. and Davis, K. R. (2000) Jasmonic acid signaling modulates ozone-induced hypersensitive cell death. Plant Cell, 12, 1633-1646.
Rieu, I. and Powers, S. J. (2009) Real-time quantitative RT-per: design, calculations, and statistics. Plant Cell Online, 21, 1031-1033.
Rouxel, T., Penaud, A., Pinochet, X., Brun, H., Gout, L., Delourme, R., Schmit, J. and Balesdent, M. H. (2003) A 10-year survey of populations of Leptosphaeria maculans in France indicates a rapid adaptation towards the Rlm1 resistance gene of oilseed rape. Eur. J. Plant Pathol. 109, 871-881.
S̆as̆ek, V., Nováková, M., Jind, B., Bóka, K., Valentová, O. and Burketová, L. (2012) Recognition of avirulence gene AvrLm1 from hemibiotrophic ascomycete Leptosphaeria maculans triggers salicylic acid and ethylene signaling in Brassica napus. Mol. Plant-Microbe Interact. 25, 1238-1250.
Schiffer, R., Görg, R., Jarosch, B., Beckhove, U., Bahrenberg, G., Kogel, K. and Schulze-Lefert, P. (1997) Tissue dependence and differential cordycepin sensitivity of race-specific resistance responses in the barley-powdery mildew interaction. Mol. Plant-Microbe Interact. 10, 830-839.
Staal, J., Kaliff, M., Bohman, S. and Dixelius, C. (2006) Transgressive segregation reveals two Arabidopsis TIR-NB-LRR resistance genes effective against Leptosphaeria maculans, causal agent of blackleg disease. Plant J. 46, 218-230.
Staal, J., Kaliff, M., Dewaele, E., Persson, M. and Dixelius, C. (2008) RLM3, a TIR domain encoding gene involved in broad-range immunity of Arabidopsis to necrotrophic fungal pathogens. Plant J. 55, 188-200.
Stotz, H. U., Mitrousia, G. K., de Wit, P. J. G. M. and Fitt B. D. L. (2014) Effector-triggered defence against apoplastic fungal pathogens. Trends Plant Sci. 19, 491-500.
Thomma, B. P. H. J., Numberger N. and Joosten, M. H. A. J. (2011) Of PAMPs and effectors: the blurred PTI-ETI dichotomy. Plant Cell 23, 4-15.
Trapnell, C., Roberts, A., Goff, L., et al. (2012) Differential gene and transcript expression analysis of RNA-seq experiments with TopHat and Cufflinks. Nat. Protoc. 7, 562-578.
Tsuda, K. and Katagiri, F. (2010) Comparing signaling mechanisms engaged in pattern-triggered and effector-triggered immunity. Curr. Opin. Plant Biol. 13, 459-465.
Van de Wouw, A. P., Cozijnsen, A. J., Hane, J. K., Brunner, P. C., McDonald, B. A., Oliver, R. P. and Howlett, B. J. (2010) Evolution of linked avirulence effectors in Leptosphaeria maculans is affected by genomic environment and exposure to resistance genes in host plants. PLoS Pathog. 6, e1001180.
Wang, Y. H. (2008) How effective is T-DNA insertional mutagenesis in Arabidopsis? J Biochem Tech, 1, 11-20.
Zhang, X., Peng, G., Kutcher, H. R., Balesdent, M. H., Delourme, R. and Fernando, W. G. D. (2016) Breakdown of Rlm3 resistance in the Brassica napus-Leptosphaeria maculans pathosystem in western Canada. Eur. J. Plant Pathol. doi:10.1007/s10658-015-0819-0.
Zheng, Z., Mosher, S. L., Fan, B., Klessig, D. F. and Chen, Z. (2007) Functional analysis of Arabidopsis WRKY25 transcription factor in plant defense against Pseudomonas syringae. BMC Plant Biol., 7, 2.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A transgenic plant comprising increased expression of a coding region encoding a resistance protein,
wherein the resistance protein is identical to or having structural similarity to a protein,
wherein the protein (i) is a receptor and comprises SEQ ID NO:22, SEQ ID NO:2, SEQ ID NO:12, SEQ ID NO:4, SEQ ID NO:6, SEQ ID NO:20, SEQ ID NO:26, or SEQ ID NO:18, (ii) is a protein involved in signal transduction and gene regulation and comprises SEQ ID NO:38, (iii) is a transcription factor and comprises SEQ ID NO:32, SEQ ID NO:34, or SEQ ID NO:36, (iv) is associated with sulfur assimilation and comprises SEQ ID NO:40 or SEQ ID NO:42, or (v) comprises SEQ ID NO:8, 10, 14, 16, 24, 28, 30, 62, 64, 66, 68, 70, 72, 74, 76, 78, 80, 82, 84, 86, 88, 90, 92, 94, 96, 98, 100, 102, 104, 106, or 108, and
wherein the amount of the resistance protein in the transgenic plant is increased compared to the wild type plant, wherein the transgenic plant is a member of the genus Brassica, and wherein the transgenic plant comprises increased resistance to infection by Leptosphaeria maculans compared to the wild type plant.

2. The transgenic plant of claim 1 wherein the transgenic plant comprises increased expression of at least two coding regions encoding resistance proteins.

3-12. (canceled)

13. The transgenic plant of claim 1 wherein the transgenic plant is B. napus, B. oleraceae, B. rapa, or B. juncea.

14. A transgenic part of the transgenic plant of claim 13 wherein the transgenic part is a leaf, a stem, a flower, an ovary, fruit, or a callus, and wherein the transgenic part comprises increased expression of a coding region.

15. A transgenic seed from the transgenic plant of claim 13.

16. Oil from the seed of claim 15.

17. Transgenic progeny of the transgenic plant of claim 13.

18. The transgenic progeny of claim 17 wherein the transgenic progeny is a hybrid plant.

19. A method of increasing resistance of a member of the genus Brassica to infection by Leptosphaeria maculans comprising increasing in the member of the genus Brassica expression of a coding region encoding a resistance protein identical to or having structural similarity to a protein, wherein the protein (i) is a receptor and comprises SEQ ID NO:22, SEQ ID NO:2, SEQ ID NO:12, SEQ ID NO:4, SEQ ID NO:6, SEQ ID NO:20, SEQ ID NO:26, or SEQ ID NO:18, (ii) is a protein involved in signal transduction and gene regulation and comprises SEQ ID NO:38, (iii) is a transcription factor and comprises SEQ ID NO:32, SEQ ID NO:34, or SEQ ID NO:36, (iv) is associated with sulfur assimilation and comprises SEQ ID NO:40 or SEQ ID NO:42, or (v) comprises SEQ ID NO:8, 10, 14, 16, 24, 28, 30, 62, 64, 66, 68, 70, 72, 74, 76, 78, 80, 82, 84, 86, 88, 90, 92, 94, 96, 98, 100, 102, 104, 106, or 108.

20. A method of making a transgenic plant with increased resistance to Leptosphaeria maculans comprising increasing expression of a coding region encoding a resistance protein identical to or having structural similarity to a protein, wherein the protein (i) is a receptor and comprises SEQ ID NO:22, SEQ ID NO:2, SEQ ID NO:12, SEQ ID NO:4, SEQ ID NO:6, SEQ ID NO:20, SEQ ID NO:26, or SEQ ID NO:18, (ii) is a protein involved in signal transduction and gene regulation and comprises SEQ ID NO:38, (iii) is a transcription factor and comprises SEQ ID NO:32, SEQ ID NO:34, or SEQ ID NO:36, (iv) is associated with sulfur assimilation and comprises SEQ ID NO:40 or SEQ ID NO:42, or (v) comprises SEQ ID NO:8, 10, 14, 16, 24, 28, 30, 62, 64, 66, 68, 70, 72, 74, 76, 78, 80, 82, 84, 86, 88, 90, 92, 94, 96, 98, 100, 102, 104, 106, or 108, wherein expression of the protein in the transgenic plant is increased compared to the wild type plant, and wherein the transgenic plant is a member of the genus Brassica.

21. A method for producing oil, comprising harvesting seeds from the transgenic plant of claim 13 and extracting the oil from the seeds.

22. A method of producing food, feed, or an industrial product comprising (a) obtaining the plant of claim 13 or a transgenic part thereof; and (b) preparing the food, feed or industrial product from the plant or transgenic part thereof.

23. The method of claim 22 wherein (a) the food or feed is oil, meal, grain, starch, flour or protein; or (b) the industrial product is biofuel, fiber, industrial chemicals, a pharmaceutical or a nutraceutical.

24. A method of producing an oil, the method comprising: (a) crushing seeds produced from at least one Brassica plant of claim 13, and (b) extracting the oil from said crushed seeds.
